(19) United States
(12) Patent Application Publication
(10) Pub. No.: US 2023/0046744 A1
(43) Pub. Date: Feb. 16, 2023

(54) METHODS AND MEANS FOR ATTRACTING IMMUNE EFFECTOR CELLS TO TUMOR CELLS

(71) Applicant: APO-T B.V., Amsterdam (NL)

(72) Inventors: Johan Renes, Amsterdam (NL); Marta Kijanka, Amsterdam (NL)

(73) Assignees: APO-T B.V., Amsterdam (NL); APO-T B.V., Amsterdam (NL)

(21) Appl. No.: 17/782,387

(22) PCT Filed: Dec. 3, 2020
(86) PCT No.: PCT/NL2020/050756

§ 371 Date: Jun. 3, 2022

(30) Foreign Application Priority Data
Dec. 4, 2019 (NL) 2024375

(51) Int. Cl. C07K 16/30; C07K 16/28; A61P 35/00; A61P 11/00

(52) U.S. Cl. CPC C07K 16/30; C07K 16/2809; A61P 35/00; A61P 11/00; C07K 2317/569; C07K 2317/31; C07K 2317/32; A61K 2039/507

(57) ABSTRACT

A first aspect of the invention relates to a method for eradicating tumor cells expressing on their surface a MHC/peptide complex comprising a peptide derived from MAGE comprising contacting said cell with at least one immune effector cell through specific interaction of a specific binding molecule for said MHC/peptide complex. Described are bispecific immunoglobulins of which one arm specifically binds to a MHC/MAGE-derived peptide complex associated with aberrant cells, and the other arm specifically recognizes a target associated with immune effector cells. The present invention relates to a pharmaceutical composition comprising such bispecific antibody and suitable diluents and/or excipients. Also a T cell comprising a T cell receptor or a chimeric antigen receptor recognizing a MHC-peptide complex comprising a peptide derived from MAGE-A is described, as well as a method of producing a T cell comprising introducing into said T cell nucleic acids encoding an α chain and a β chain or a chimeric antigen receptor.

### TECHNICAL FIELD

The invention relates to the field of biotherapeutics. It particularly relates to the field of tumor biology. More in particular the invention relates to the field of molecules capable of attracting immune effector cells to aberrant cells in cancers. The invention also relates to such molecules targeting aberrant cells and attracting immune effector cells, while leaving normal cells essentially unaffected. More in particular, the invention relates to specific binding molecules comprising binding domains specific for at least two different binding sites, one being on the surface of aberrant cells, and the other on the surface of immune effector cells. The invention also relates to the use of these specific binding molecules in selectively killing cancer cells.

### BACKGROUND

Cancer is caused by oncogenic transformation in aberrant cells which drives uncontrolled cell proliferation, leading to misalignment of cell-cycle checkpoints, DNA damage and metabolic stress. These aberrations should direct tumor cells towards an apoptotic path which has evolved in multi-cellular animals as a means of eliminating abnormal cells that pose a threat to the organism. Indeed, most transformed cells or tumorigenic cells are killed by apoptosis. However, occasionally a cell with additional mutations that enable avoidance of apoptotic death, survives thus enabling its malignant progression. Thus, cancer cells can grow not only due to unbalances in proliferation and/or cell cycle regulation, but also due to unbalances in their apoptosis machinery. Unbalances like, for example, genomic mutations resulting in non-functional apoptosis inducing proteins or over-expression of apoptosis inhibiting proteins form the basis of tumor formation. Fortunately, even cells that manage to escape the apoptosis signals this way when activated by their aberrant phenotype, are still primed for eradication from the organism. Apoptosis in these aberrant cells can still be triggered upon silencing or overcoming the apoptosis inhibiting signals induced by mutations. Traditional cancer therapies can activate apoptosis, but they do so indirectly and often encounter tumor resistance. Direct and selective targeting of key components of the apoptosis machinery in these aberrant cells is a promising strategy for development of new anti-tumor therapeutics. Selective activation of the apoptosis pathway would allow for halting tumor growth and would allow for induction of tumor regression.

A disadvantage of many if not all anti-tumor drugs currently on the market or in development, is that these drugs do not discriminate between aberrant cells and healthy cells. This non-specificity bears a challenging risk for drug induced adverse events. Examples of such unwanted side effects are well known to the field: radiotherapy and chemotherapeutics induce cell death only as a secondary effect of the damage they cause to vital cellular components. Not only aberrant cells are targeted, though in fact most proliferating cells including healthy cells respond to the apoptosis-stimulating therapy. Therefore, a disadvantage of current apoptosis inducing compounds is their non-selective nature, which reduces their potential.

Since the sixties of the last century it has been proposed to use the specific binding power of the immune system (T cells and antibodies) to selectively kill tumor cells but leave alone the normal cells in a patient's body. The introduction of monoclonal antibodies (mAb) has been a great step in bringing us closer towards personalized and more tumor specific medicine. However, one of the major challenges, being the design of a therapy that is at the same time efficacious and truly cancer-specific, still remains unresolved. The majority of mAbs currently approved by the US Food and Drug Administration and undergoing evaluation in clinical trials target cell surface antigens, more rarely to soluble proteins [Hong, C. W. et al Cancer Res, 2012. 72 (15): p. 3715-9; Ferrone, S., Sci Transl Med, 2011. 3 (99): p. 99.]. These antigens represent haematopoietic differentiation antigens (e.g. CD20), glycoproteins expressed by solid tumors (e.g. EpCAM, CEA or CAIX), glycolipids (i.e. gangliosides), carbohydrates (i.e. Lewis Y antigen), stromal and extracellular matrix antigens (e.g. FAP), proteins involved in angiogenesis (e.g. VEGFR or integrins), receptors involved in growth and differentiation signaling (e.g. EGFR, HER2 or IGF1R). For essentially all of these antigens expression is associated with normal tissue as well. Thus, so far selective killing of aberrant cells has been an elusive goal.

Proteins of the Melanoma Antigen Gene family (MAGE) were the first identified members of Cancer Testis antigens (CT). Their expression pattern is restricted to germ cells of immuno-privileged testis and placenta, as well as a wide range of malignant cells. Expression of CT antigens in cancer cells was shown to result in their uncontrolled growth, resistance to cell death, potential to migrate, grow at distant sites and the ability to induce growth of new blood vessels (Morten F. Gjerstorff et al., Oncotarget, 2015.6 (18): p. 15772-15787; Scanlan M J, G. A. et al. Immunol Rev., 2002. 188: p. 22-32). Due to their intracellular expression MAGE proteins remain inaccessible targets until they undergo proteasomal degradation into short peptides in the cytoplasm. These peptides generated by the proteasome are then transported into endoplasmic reticulum where they are loaded onto the Major Histocompatibility Complex (MHC) class I molecules. Intracellularly processed MAGE-derived peptides can be used as an immunotherapy target once present on the cell membrane in complex with MHC class I molecules. The MHC molecules present the MAGE derived peptides to specialized cells of the immune system. The few cells that do not express MHC class I molecules are the cells from testis and placenta. Therefore normal cells that express MAGE protein do not have the MHC class I molecules, and the normal cell that have MHC class I molecules do not have the MAGE protein. The MAGE derived peptides in context of MHC class I are therefore truly tumor specific targets. Targeted therapeutics specifically binding MAGE peptides in context of MHC-I are therefore expected not to elicit off-tumor on-target toxicity and thereby have an improved safety profile.

One of the subsets of immune effector cells are NK cells. Due to expression of CD16 on their surface they are capable of recognition and binding of Fc parts of immunoglobulins. Upon binding of Fc region of an IgG to Fc receptor NK cells release cytotoxic factors that cause the death of the cell bound by the IgG. These cytotoxic factors include perforin and granzymes, a class of proteases, causing the lysis of aberrant cell. Such mode of attracting immune effector cells is referred to as ‘antibody dependent cell-mediated cytotoxicity’. It is of course also possible, and in fact preferable, to have the second arm of the bispecific antibody recognize the CD16 and disable the Fc part of the bispecific antibody.

Another subset of immune effector cells are T cells. T cells originate in the bone marrow and undergo maturation in the thymus, where they multiply and differentiate into helper, regulatory, or cytotoxic T cells or become memory T cells. Upon maturation T cells circulate in the blood or lymphatic system or localize at peripheral tissues. Cytotoxic T lymphocytes (CTL) are responsible for destruction of virus-infected and malignant cells through the induction of apoptosis. Apoptosis, also referred to as programmed cell death, is a process characterized by a number of cellular changes such as transfer of phosphatidylserine to the outer leaflet of the plasma membrane, caspase activation, disruption of the inner mitochondrial transmembrane potential, cell shrinkage and membrane blebbing, chromatin condensation and DNA fragmentation. Death by apoptosis does not result in release of cellular contents. In order to reduce inflammation, the cell breaks into fragments that are subsequently removed by phagocytes.

As a T cell scans the surface of a target cell (virus-infected cell or tumor cell) to find a specific MHC/peptide complex, the process of immune synapse formation begins when the T cell receptor (TCR) present on the T cell surface binds to the MHC/peptide complex presented on the target cell surface. This initiates a signaling activation lead to polarization of the T cell. In case of CTL, the synapse formation leads to killing of the target cell via secretion of cytolytic enzymes.

The CTLs contain lytic granules (specialized secretory lysosomes) which hold pore-forming proteins such as perforins and proteolytic enzymes called granzymes, as well as lysosomal hydrolases (for example cathepsins B and D, β-hexosaminidase). When the T Cell Receptor Complex and CD8 expressed on the surface of the CTL bind to the MHC/peptide complex presented on the surface of the virus-infected or tumor cell, a CD3 molecule mediated signaling cascade is activated. This activation triggers the release of the perforins, granzymes and chemokines. The perforin molecules polymerize and form pores in the membrane of the virus-infected or tumor cell. This leads to increased permeability of the said cell and activation of the caspase-dependent apoptotic cascade. Furthermore, formation of these pores is needed to allow granzymes to enter the cell recognized and bound by TCL. Infusion of granzymes results in (i) caspase enzymes activation leading to apoptosis, (ii) destruction the cytoskeleton, (iii) degradation of the cell's nucleoproteins and (iv) activation of enzymes that degrade DNA. Another mechanism for CTL-mediated programmed cell death is dependent on FasL (Fas Ligand)/Fas receptor interactions. In this process as a consequence of caspase-8 activation, the downstream caspases, such as caspase-3, -6, and -7 are activated and lead to apoptosis. The death signal can also be initiated by the release of mitochondrial cytochrome-C and activation of Apoptotic Protease-Activating Factor-1 (APAF1). Moreover, the autolytic activation of caspase-9 may initiate the effector caspase cascade resulting in DNA fragmentation.

Attracting of immune effector cells, such as T cells, to aberrant cells can be done by (retroviral) introduction of chimeric T cell receptors (cTCRs) or chimeric antibody receptors (CARs) providing specificity to markers expressed on the cell surface of aberrant cells. Chimeric TCRs have been so far generated by fusing an antibody derived V_{H} and V_{L} chain to a TCR Cβ and Cα chain, respectively. T cells expressing these cTCRs have been described to show specific functionality in vitro (Gross, G et al. Proceedings of the National Academy of Sciences, 1989. 86 (24): p. 10024-10028). One of the advantages of this format over the CAR format would be that the intracellular signaling in T cells expressing cTCRs occurs via the natural CD3 complex, in contrast to the signaling in CAR expressing T cells. Multiple clinical studies using TCR and CAR engineered T cells have shown promising results (Brentjens, R. J., et al. Science translational medicine, 2013. 5 (177); Robbins, P. F., et al. Clinical Cancer Research, 2015. 21 (5): p. 1019-1027; Porter, D. L., et al., Science translational medicine, 2015. 7 (303)).

CARs represent the same principle of attracting immune effector cells to aberrant cells as chimeric TCRs, however the molecule format differs. Three generations of CARs have been developed so far. First-generation CARs consist of antibody derived V_{H} and V_{L} chains in a so-called single-chain (scFv), or Fab format which are fused to a CD4 transmembrane domain and a signaling domain derived from one of the proteins within the CD3 complex (e.g.: ξ, γ). To improve CAR T cell function and persistence, second generation CARs were developed which contain one co-stimulatory endodomain derived from for instance CD28, OX40 (CD134) or 4-1BB (CD137). Third generation CARs harbor two co-stimulatory domains (Sadelain, M. et al. Cancer discovery, 2013. 3 (4): p. 388-398). For long, the use of CAR T-cell therapy has been restricted to small clinical trials, mostly enrolling patients with advanced blood cancers. The two lately approved by FDA therapies include one for the treatment of children with acute lymphoblastic leukemia (Kymriah by Novartis Pharmaceuticals Corporation) and the other for adults with advanced lymphomas (Yescarta by Kite Pharma, Incorporated). Both of these employ CD19 molecule, also present on healthy B-cells, as tumor marker. Targeting solid tumors remains, however, a big challenge in the field of immuno-oncology. The main underlying reasons are low T cell infiltration and the immunosuppressive environment that tumor cells create to evade immune cells.

Another possibility to attract immune effector cells to the tumor site is the use of bispecific molecules, e.g. bispecific antibodies. Bispecific molecules are being developed as cancer therapeutics in order to (i) inhibit two cell surface receptors, (ii) block two ligands, (iii) cross-link two receptors or (iv) recruit immune cells which do not carry a Fc receptor (such cells are not activated by antibodies). Over time several ways of production of bispecific molecules have been developed. First bispecific molecules were produced either by reduction and re-oxidation of cysteines in the hinge region of monoclonal antibodies. Another option was to produce bispecific molecules by fusion of two hybridomas. Such fusion resulted in formation of a quadroma, from which a mixture of IgG molecules is produced. Such production system provides, however, limited amount of actual bispecific molecules. Chimeric hybridomas, common light chains and recombinant proteins addressed the limitation of proper antibody light and heavy chain association in order to generate a bispecific molecule. The heavy-light chain pairing in chimeric quadromas is species restricted. Advances in the field of recombinant DNA technology opened up new opportunities regarding composition and production systems of bispecific molecules. The correct bispecific molecule structure in a recombinant protein can be ensured by employing various strategies, such as e.g. knobs-in-holes approach (one heavy chain is engineered with a knob consisting of relatively large amino acids, whereas the other is engineered with a hole consisting of relatively small amino acids) or connecting antibody fragments as peptide chains to avoid random association of the chains (e.g. connecting two single chain variable fragments of different specificities by a linker as employed in the BiTE® approach). Bispecific molecules can be categorized based on their structure into IgG-like molecules, which contain an Fc region, or non-IgG like which lack the Fc region. IgG-like bispecific molecules are bigger in size and have longer half-life in serum, whereas non-IgG like antibodies have a smaller size which allows for better tumor penetration, however exhibit a much shorter serum half-life. Availability of numerous formats of bispecific molecules allows for modulation of their immunogenicity, effector functions and half-life.

Growing interest in immune-oncology resulted in the development of immune cell engaging molecules/antibodies. Examples of such bispecific molecules, of which one binding arm recognizes a target expressed on the surface of a tumor cell and the second arm an antigen present on the effector immune cells, such as for example CD3 on T cells have been described (Kontermann R E, MAbs. 2012, 4(2):182-97; Chames P. et al. MAbs. 2009, 1(6):539-47; Moore P. A. et al. Blood. 2011, 117(17):4542-51). The so-called trio mAb CD3×Epcam bispecific antibody, also known as catumaxomab, has been developed clinically and has been registered in Europe for palliative treatment of abdominal tumors of epithelial origin. Catumaxomab binds EpCAM positive cancer cells with one antigen binding arm and the T-cell antigen CD3 with the other (Chelius D. et al, MAbs. 2010, 2(3):309-19). In addition to the direction of T cells towards the EpCAM positive cancer cells via the CD3 binding, this approach also facilitates the binding of other immune cells, e.g. natural killer cells and macrophages by the Fc domain of this antibody rendering this strategy bi-specific but tri-functional. The widespread application of this format is however prevented by its rodent nature, which induces anti-product immune responses upon repetitive dosing.

Alternative formats for molecules redirecting immune effector cells to cancer sites have been evaluated such as Dual-Affinity Re-Targeting (DART™) molecules that are developed by Macrogenics, Bispecific T cell Engager (BiTE®) molecules that were developed by Micromet, now Amgen (Sheridan C, Nat Biotechnol. 2012 (30):300-1), Dual Variable Domain—immunoglobulin (DVD-Ig™) molecules that are developed by Abbott, and TandAb® RECRUIT molecules that are developed by Affimed. Up to date the cancer related antigens targeted by these formats are not truly tumor specific as in case of MAGE antigen. The CD3×CD19 BiTE®, blinatumomab, has demonstrated remarkable clinical efficacy in refractory non-Hodgkin lymphoma and acute lymphatic leukemia patients (Bargou R. et al. Science. 2008, 321(5891): 974-7). One of the targets recognized by blinatumomab is CD19, a cell surface antigen expressed on both neoplastic and healthy B-cells. The results of Blinatumomab spiked the development of various molecules directing T-cell activity towards tumour sites. Some of these molecules, recognizing tumor associated but not tumor specific targets such as EpCAM, CD33, ErbB family members (HER2, HER3, EGFR), death receptors (such as CD95 or CD63), proteins involved in angiogenesis (such as Ang-2 or VEGF-A) or PSMA, are currently undergoing clinical evaluation (Krishanumurthy A. et al., Pharmacol Ther. 2018 May; 185:122-134).

There thus remains a need for effective specific binding molecules capable of recognizing a target exclusively accessible on the surface of aberrant cells and recruiting immune effector cells to such cells without being immunogenic.

### SUMMARY OF THE INVENTION

A first aspect of the present invention relates to a bispecific molecule of which one arm comprises a first domain that specifically binds to a MHC/peptide complex comprising a peptide derived from MAGE expressed on the cell surface of aberrant cells, and the other arm comprises a second domain that specifically recognizes a target expressed on the cell surface of immune effector cells.

With such bispecific molecules it has now become possible to bind and bring together the aberrant cells expressing MAGE and immune effector cells, such as T-cells (CTLs) and NK cells. This way apoptosis is efficiently induced in said aberrant cells.

The first and second domains of the bispecific molecules of the present invention are preferably VH, VHH or VL domains. In a specific embodiment said domains are preferably both VHH domains. Although various options are available for preparing these bispecific molecules, they are preferably in a BiTE format, as is well known to the person skilled in the art.

The first domain of the bispecific molecules may have a VHH domain according to any of the following sequences: SEQ ID NO: 47; SEQ ID NO: 48; SEQ ID NO:49; SEQ ID NO: 50; SEQ ID NO: 51; SEQ ID NO: 52; SEQ ID NO: 53; SEQ ID NO: 54; SEQ ID NO: 55; or SEQ ID NO: 56.

The first domain of the bispecific molecules also may have a VH domain according to any of the following 46 sequences: SEQ ID NO: 1-46.

The target to be recognized on the effector cells (such as T-cells and NK-cells) is preferably CD3. Hence, the second domain of the bispecific molecules of the present invention bind well to said protein. Said second domain is preferably a VHH domain according to SEQ ID NO:57. Said second domain for binding to CD3 is also preferably a VH domain consisting of or comprising the amino-acid sequence DIKLQQSGAELARPGASVKMSCKTSGYTFTRYTMHWVKQRPGQGLEWIGYINPSRGYTNYNQKFKDKA TLTTDKSSSTAYMQLSSLTSEDSAVYYCARYYDDHYCLDYWGQGTTLTVSS (SEQ ID NO: 66).

An embodiment is the bispecific molecule comprising the VHH of the invention, fused (such as for example covalently linked via peptide bonds) with an Fc tail of an antibody.

An embodiment is the bispecific molecule comprising the VH of the invention, fused (such as for example covalently linked via peptide bonds) with an Fc tail of an antibody, wherein optionally the bispecific molecule also comprises a VL domain for binding to the VH domain, therewith providing e.g. an IgG antibody format.

A further aspect of the present invention relates to a pharmaceutical composition comprising the above mentioned bispecific molecules. Said composition may comprises diluents (such as water) and excipients commonly known in the art. In this regard reference is made to the Rowe et al., Handbook of Pharmaceutical excipients.

A further embodiment of the invention is the use of combinations of bispecific molecules according to the invention, as well as pharmaceutical compositions for said use and methods of treatment comprising combinations of bispecific molecules according to the invention. One of the most prominent problems in tumor therapy is escape from therapy. It is more difficult for a tumor to down regulate several molecules at the same time. Therefore the combination therapy according to the present invention makes escape more difficult. If all MHC classes on a cell are targeted by at least one bispecific molecule according to the invention at essentially the same time, the targeted tumor cells must downregulate all MHC-1 thereby becoming an NK cell target. It is also possible to provide bispecifics with the same MHC-1 peptide specificity but different T-cell binders. Combinations of these approaches are also possible. It is best to be able to treat the tumor with a first combination comprising several bispecifics according to the invention and to be able to follow up with a different combination if the tumor downregulates the targets for the first combination. As there are at least three, but possibly six different MHC-1 variations on a cell and each may present different peptides form different MAGE proteins, there will typically be several combinations available. Diagnosis of a patient tumor's HLA status and MAGE status is therefore a part of this invention to determine which bispecifics and in particular which combinations can be administered to a particular patient.

A further aspect of the present invention relates to the use of the bispecific molecules and pharmaceutical formulations thereof in the treatment of cancer, in particular lung cancer, more in particular small cell lung cancer. According to the invention, the bispecific molecules and pharmaceutical formulations thereof are suitable for use in a regimen for the treatment of cancer, wherein the tumor cell expresses at least one MAGE, preferably at least one MAGE-A. Examples are solid tumors and hematological malignancies, wherein target cells express a MAGE. Expression of at least one MAGE in the tumor cell ensures the exposure of at least one MHC/MAGE peptide complex on the tumor cell surface, such as at least one HLA/MAGE-A peptide complex.

An aspect of the invention relates to a method for eradicating tumor cells expressing on their surface a MHC-peptide complex comprising a peptide derived from MAGE, the method comprising contacting said cell with at least one immune effector cell through specific interaction of a specific binding molecule for said MHC-peptide complex, wherein said specific binding molecule is a bispecific molecule for binding to an MHC/MAGE peptide complex and to CD3, wherein the bispecific molecule comprises a VH domain for binding to an MHC/MAGE peptide complex selected from HLA complexed with any of MAGE-A peptides and comprises a VH domain for binding to a CD3.

### BRIEF DESCRIPTION OF THE DRAWINGS

FIG. 1—Specificity of A09 immunoglobulin was assessed in a flow cytometric assay employing a panel of cells of different origin. H1299 cells are HLA-A2 negative, MAGE-A positive and serve as a negative control, H1299_A2/mMA are stably expressing HLA-A2/mMA complexes and serve as a positive control. U87 cells (HLA-A2 positive, MAGE positive) are of glioblastoma origin, 911 cells (HLA-A2 positive, MAGE negative) are derived from embryonic retinoblasts. (A) The binding of A09 was detected in a flow cytometric assay. The A09 IgG bound specifically to HLA-A2+, MAGE+ cells (U87, H1299_A2/mMA), however not to HLA-A2+, MAGE− cells (911) or HLA-A2−, MAGE+ cells (H1299). (B) The HLA-A2 expression status of used cell lines was assessed by flow cytometric staining using anti-HLA-A2-BB515 antibody.

FIG. 2—Binding characteristics of purified mouse 9A7 IgG established in ELISA on recombinant HLA/MAGE-derived peptide complexes (A) and in flow cytometric assay employing a panel of cell lines (B).

FIG. 3—Assessment of fine specificity of HLA/MAGE-derived peptide specific antibodies: (A) 4A6 IgG, (C) A09 IgG, (E) chimeric 9A7 IgG. Assessment of IgG binding was performed using peptide pulsed JY cells (A and C) or peptide pulsed K562 cells stably expressing HLA-A*2402 (E). The tables in (B) and (D) present the sequences of respective peptides used to assess fine specificity of tested IgG molecules, MAGE-A peptide origin and peptide affinity to HLA-A*0201 [nM]. Table in (F) presents the sequences of respective peptides used to assess fine specificity of tested chimeric IgG molecule, MAGE-A peptide origin and peptide affinity to HLA-A*2402 [nM].

FIG. 4—Transduced T cells express MAGE/HLA-A2 specific CAR on their surface. T cells transduced with scFv 4A6 CAR pMx-puro vector and control T cells transfected with pMx-puro vector were subjected to flow cytometric staining using tetramers of HLA-A2/MA_{3, 12} (FLWGPRALV)-PE. The tetramers were produced by mixing biotinylated HLA-A2/MA_{3, 12} complexes with PE streptavidin at a molar ratio 5:1. Samples were incubated at 4° C., in the dark for 30 minutes. Detection of CD8 positive T cells was performed using the APC Mouse Anti Human CD8 (4A and 4B), whereas to detect the CD4 T cells, FITC Mouse Anti Human CD4 Antibody was used (4A, bottom panel).

FIG. 5—Granzyme B release as effect of T cell activation. scFv 4A6 CAR T cells (B) or pMx-puro-RTV 014 T cells (A) were co-incubated with T2 cells pulsed with MA3 (relevant, FLWGPRALV) or MA1 (irrelevant) peptides. lonomycin was used as a positive control for T cell activation. Cells were stained extracellularly with anti-human CD8 (A, B left column) and CD4 (A, B right column), followed by intracellular staining with anti-human granzyme B (y axis: granzyme:PE, x axis: CD8/CD4).

FIG. 6—Purification and specificity of bispecific molecules. (A) Bispecific molecules 4A6×CD3, A09×CD3 and CD19×CD3 were expressed in mammalian cells and purified from cell culture medium using Talon beads. Purity of elution fractions was assed using a stain free SDS-PAGE gel. (B) Purity of the bispecific molecules was assessed after de-salting step using stain free SDS-PAGE. (C) 4A6×CD3 specifically binds HLA-A2/MA_{3, 12} (black squares) and not HLA-A2/mMA (black circles) in ELISA on biotinylated peptide/HLA complexes. (D) 4A6×CD3 binds PBMCs from healthy donors (indicated by shift of MFI signal in bottom histogram when compared to upper histogram that serves as a background reference). Negative control molecule 4A6_SC_FV did not bind PBMC (as indicated by lack of shift in middle histogram when compared to upper histogram that serves as a background reference). (E) Alanine scanning analysis of 4A6×CD3 fine specificity. (F) Table showing amino acid sequences of peptides used in the alanine scanning experiment, as well as their predicted affinity to HLA-A2 molecule. Random peptide is used as a control peptide with high affinity towards HLA-A2. It is a negative control as 4A6×CD3 does not carry fine specificity towards this peptide/HLA complex.

FIG. 7—T-cell activation by the bispecific molecule of the disclosure in context of H1299 cells expressing target MAGE-A derived peptide/HLA complex. (A) 72 hours incubation of 500 ng/ml 4A6×CD3 (BiTE A) with H1299 expressing HLA-A2/MA_{3, 12} cells (Target A) and 72 hours incubation of 500 ng/ml A09×CD3 (BiTE B) with H1299 expressing HLA-A2/mMA cells (Target B) in presence of PBMC leads to increase of percentage of CD69 positive T cells. (B) 72 hours incubation of 500 ng/ml 4A6×CD3 (BiTE A) with H1299 expressing HLA-A2/MA_{3, 12} cells (Target A) and 72 hours incubation of 500 ng/ml A09×CD3 (BiTE B) in with H1299 expressing HLA-A2/mMA cells (Target B) in presence of PBMC leads to increase of percentage of CD25 positive T cells. (C) Representative histograms showing the mean fluorescent intensity (MFI) of T cells incubated with target cells as indicated in the FIGS. 7A and 7B either without bispecific molecule 4A6×CD3 (upper histogram) or in presence of bispecific molecule 4A6×CD3 (middle histogram) or A09×CD3 (bottom histogram). (D) Dose dependent increase in CD69 expression of T cells with increasing amounts of bispecific molecule. (E) Different target- to effector-cell ratios did not affect the percentage of CD69 positive T cells when incubated with either 4A6×CD3 on H1299 HLA-A2/MA_{3, 12} or A09×CD3 on H1299 HLA-A2/mMA cells. (F) Physical attraction of PBMC to H1299 expressing HLA-A2/MA_{3, 12} cells in presence of 4A6×CD3 after 24 hour incubation. (G) 4A6×CD3 and A09×CD3 facilitate PBMC interactions with H1299 cells expressing respectively HLA-A*0201/MA_{3, 12} (annotated in the Figure as H1299 HLA-A2/FLWGPRALV) or HLA-A*0201/mMA (annotated in the Figure as H1299 HLA-A2/YLEYRQVPG) after 72 hours incubation. Exemplary interactions are indicated with black arrows.

FIG. 8—T-cell activation by the bispecific molecule of the disclosure in context of 911 cells expressing target MAGE-A derived peptide/HLA complex. (A) 72 hours incubation of 500 ng/ml 4A6×CD3 with 911 cells expressing HLA-A2/MA_{3, 12} complex leads to increase of percentage of CD69 positive T cells. (B) 72 hours incubation of 500 ng/ml 4A6×CD3 with 911 cells expressing HLA-A2/MA_{3, 12} complex leads to increase of percentage of CD25 positive T cells. (C) Representative histograms showing the mean fluorescent intensity (MFI) of T cells incubated with target cells as indicated in the FIGS. 8A and 8B either without bispecific molecule 4A6×CD3 (upper histograms) or in presence of bispecific molecule 4A6×CD3 (bottom histograms). (D) Different target-to-effector cell ratios did not affect the percentage of CD69 positive T cells when incubated with 4A6×CD3 in presence of 911 cells expressing HLA-A2/MA_{3, 12} complexes. (E) Representative images showing the decreased number of 911 cells expressing HLA-A2/MA_{3, 12} complexes upon 72 hours incubation with 4A6×CD3 and PBMCs. (F) Both 4A6×CD3 and A09×CD3 molecules of the disclosure induced PBMC interactions with 911 cells expressing respective target HLA/MAGE-A derived peptide complexes. Phase contrast images were taken after 48 hours incubation of respective BiTE molecule of the disclosure with co-cultured PBMCs and stably transfected 911 cells. 4A6×CD3 induced PBMC interactions with 911 cells stably expressing HLA-A*0201/FLWGPRALV complexes (annotated in the Figure as 911 HLA-A2/FLWGPRALV), whereas A09×CD3 induced PBMC interactions with 911 cells stably expressing HLA-A*0201/YLEYRQVPG complexes (annotated in the Figure as 911 HLA-A2/YLEYRQVPG). These interactions are specific and BiTE-dependent.

FIG. 9—T-cell activation upon incubation with A09×CD3 and glioblastoma cells. (A) Specific increase in percentage of CD69 positive T cells was observed when PBMCs were incubated for 72 hours with 4A6×CD3 or A09×CD3 molecules in presence of U87 cells. (B) Representative histograms showing the mean fluorescent intensity (MFI) of CD69 positive T cells upon incubation with U87 cells either without bispecific molecule (upper histograms) or in presence of bispecific molecule (bottom histograms).

FIG. 10—Detection of apoptotic marker (cleaved PARP) in lysates of cells expressing respective HLA/MAGE-derived peptides co-cultured with PBMC in presence of BiTE molecules of the disclosure. An increase in cleaved PARP presence is observed in lysate samples of H1299 stably expressing HLA-A*0201/FLWGPRALV complexes (annotated in the Figure as H1299 HLA-A2/FLWGPRALV) (A, upper panel) and H1299 stably expressing HLA-A*0201/YLEYRQVPG (annotated in the Figure as H1299 HLA-A2/YLEYRQVPG) (A, bottom panel) upon co-incubation with PBMCs and 4A6×CD3 or A09×CD3, respectively. (B) Lysates of human pulmonary fibroblasts (HPF) co-incubated with PBMCs and either 4A6×CD3 or A09×CD3 did not result in the detection of cleaved PARP by Western blot.

FIG. 11—BiTE molecules of the disclosure induced HLA/MAGE-derived peptide complex specific reduction of target cell numbers as a result of apoptosis induction. (A) H1299 cells stably expressing HLA/MAGE-derived peptide complexes of interest were co-cultured for 3 days with PMBC (target to effector ratio 1:16) in presence or absence of respective BiTE molecules of the disclosure (at 500 ng/ml). Decrease in number of target cells was observed only in conditions in which PBMC were co-cultured either with H1299 stably expressing HLA-A*0201/FLWGRPALV (annotated in the Figure as H1299 HLA-A2/FLWGPRALV) in presence of 4A6×CD3, or with H1299 stably expressing HLA-A*0201/YLEYRQVPG (annotated in the Figure as H1299 HLA-A2/YLEYRQVPG) in presence of A09×CD3. (B) Increasing activation of caspase 3/7 in time was detected during live cell imaging by Incucyte only in condition in which 911 cells expressing HLA-A*0201/FLWGRPALV (annotated in the Figure as 911 HLA-A2/FLWGPRALV) were co-cultured with PBMC in presence of 4A6×CD3. (C) Detected activation of caspase 3/7 was dependent on concentration of BiTE. (D) Live cell imaging of PMBCs targeting 911 cells expressing HLA-A2/FLWGPRALV in presence of 4A6×CD3 was shown in a time-course microscopy experiment.

FIG. 12—Target specific BiTE induction of IL-6, IL-10, TNF and INF-γ release by PMBCs. Both (A) A09×CD3 and (B) 4A6×CD3 are capable of inducing cytokine release by PBMCs in the presence of HLA-A*0201/FLWGPRALV or HLA-A*0201/YLEYRQVPG expressing H1299 cells (annotated in the Figure as HLA-A2/FLWGPRALV and HLA-A2/YLEYRQVPG), respectively, after 72 hours of incubation.

FIG. 13—4A6×CD3 leads to reduced tumor growth H1299 xenografts expressing HLA-A*0201/FLWGPRALV in a mouse model harboring a human immune system (annotated in the Figure as humanized). Tumor growth was slowed down in animals dosed with 4A6×CD3 (squares) compared to tumor growth in mice dosed with vehicle (circles).

FIG. 14—Purification of bi-specific nanobody construct. Expressed nanobody present in the periplasmic fraction (P) after purification was no longer detectable in the flow through (F) and could be efficiently eluted from the purification beads (E). Elution fractions were pooled and desalted (DE).

FIG. 15—Bispecific nanobody 1B10×CD3, in which the N-terminal nanobody binds HLA-A*0201/YLEYRQVPG complex presented on the surface of tumor cells and is connected via a G4S linker to C-terminal nanobody which binds CD3 expressed on the surface of immune effector cells, facilitated formation of immune synapses between effector cells and target cells. These interactions are shown by black arrows.

FIG. 16—9A7×CD3 induces PBMC activation and reduces target cell number in a target specific manner. (A) Immune effector cells co-incubated with K562 cells expressing HLA-A*2402/IMPKTGFLI (annotated in Figure as K652 HLA-A24/IMPKTGFLI) in presence of 9A7×CD3 showed an increase in expression of early T cell activation marker CD69 after 24 hour incubation. (C) Immune effector cells co-incubated with K562 cells expressing HLA-A*2402/IMPKTGFLI in presence of 9A7×CD3 showed an increase in expression of late T cell activation marker CD25 after 72 hour incubation. A decrease in number of K562 cells expressing HLA-A*2402/IMPKTGFLI was observed, when cells were co-incubated with PBMCs and 9A7×CD3 for 24 hours (B) and 72 hours (D).

FIG. 17—Incubation with 9A7×CD3 leads to T cell activation and reduced target expressing H1299 cells numbers. (A) Late T cell activation marker CD25 was increased after 72 hours of incubation with 9A7×CD3 and H1299 cells (MAGE-A and HLA-A*2402 positive) (B). Significant decrease of H1299 cell number was observed upon co-incubation with PBMC in presence of BiTE molecule of the disclosure. Decrease in number of control cell lines U87 and human pulmonary fibroblasts was not observed. (C) An increase in expression of late T cell activation marker CD25 is observed when PBMC are co-cultured in presence of 9A7×CD3 BiTE with K562 HLA-A24/IMPKTGFLI, U118 and H1299 cells.

FIG. 18—BiTE of the disclosure, the 9A7×CD3, induces PBMC interactions with target expressing cells (namely HLA-A*2402/IMPKTGFLI). Interactions between PMBC and K562 cells expressing HLA-A*2402/IMPKTGFLI (annotated in the Figure as K562 HLA-A24/IMPKTGFLI) in presence of 9A7×CD3 are indicated with black arrows.

FIG. 19—BiTE of the disclosure, the 9A7×CD3, after 4 hours of incubation induced PBMC interactions with H1299 and to lesser extend U118 (HLA-A*2402 and MAGE positive), but not with U87 (HLA-A*2402 negative, MAGE-A positive) or human pulmonary fibroblasts (HLA-A*2402 positive). The interactions are shown by black arrows.

FIG. 20—BiTE of the disclosure, the 9A7×CD3, after 24 hours of incubation induced PBMC interactions with H1299 and U118 (HLA-A*2402 and MAGE positive), but not with U87 (HLA-A*2402 negative, MAGE-A positive) or human pulmonary fibroblasts (HLA-A*2402 positive). The interactions are shown by black arrows.

FIG. 21—Specific binding of phage display selected Fab fragments to HLA-A2/mMA complexes (data shown in upper table). As a positive control AH5 Fab (produced from pCES vector) and AH5 monoclonal IgG were used. Clones showing binding to HLA-A2/MA3 complexes (data shown in bottom table) are considered to not carry the desired fine specificity.

FIG. 22—Assessment of fine specificity of 9A7×CD3. (A) Assessment of binding was performed using peptide pulsed HEK-293F cells expressing HLA-A*2402, following the alanine scanning approach. The table in (B) present the sequences of respective peptides used to assess fine specificity of 9A7×CD3 and peptide affinity to HLA-A*2402 [nM].

FIG. 23—9A7×CD3 induced T cell activation towards MAGE-A HLA-A*2402 expressing cells. (A, B) Activation and degranulation of CD4 and CD8 positive cells when co-cultured for 16 hours with DLD-1, U118 and H1299 cells in the presence of 5 ng/ml 9A7×CD3. BiTE induced expression of CD69, CD25 and CD107a on CD4 and CD8 positive cells is observed when co-cultured for 16 hours with HLA-A*2402 and MAGE-A positive cells. (C, D) Activation and degranulation of CD4 and CD8 positive cells when co-cultured for 72 hours with DLD-1, U118 and H1299 cells in the presence of 5 ng/ml 9A7×CD3. 9A7×CD3 induced expression of CD69 and CD25 on CD4 and CD8 positive cells is observed when co-cultured for 72 hours with HLA-A*2402 and MAGE-A positive cells. After 72 hours, CD107a expression is only observed on CD8 positive cells in the presence of HLA-A*2402 and MAGE-A positive U118 cells.

FIG. 24—Cytokine release by PBMCs upon co-culturing DLD-1 (A), U118 (B) and H1299 (C) with or without 5 ng/ml 9A7×CD3 for 72 hours. IFN-γ, IL-2 and IL-6 are secreted by PBMCs in the presence of H1299 and U118. The presence of H1299 also resulted in the release of IL-10 secretion whereas co-culturing of U118 showed increased levels of TNF. No BiTE induced cytokine release was observed when co-culturing PBMCs with DLD-1 cells.

FIG. 25—Cleaved parp detection using Western blot in H1299, U118 and DLD-1 protein lysate samples upon incubation with PBMC's in combination with 9A7×CD3 for 72 hours. Cleaved PARP was only detected when H1299 cells were incubated with PBMCs and 9A7×CD3 as indicated in figure.

### DISCLOSURE OF THE INVENTION

It is a goal of the present invention to attract immune effector cells specifically to tumor cells. A second goal is to provide a pharmaceutically active molecule that facilitates specific and effective induction of aberrant cell's death. In particular, it is a goal of the present invention to specifically and selectively target aberrant cells and induce apoptosis of these aberrant cells, leaving healthy cells essentially unaffected. MHC-1 peptide complexes on tumors of almost any origin are valuable targets, whereas MHC-2 peptide complexes are valuable targets on tumors of hematopoietic origin. In this application we will typically refer to MHC-I. Of course in most of the embodiments MHC-II may be used as well, so that MAGE/MHC-II peptide complexes are also part of the invention.

An aberrant cell is defined as a cell that deviates from its healthy normal counterparts. Aberrant cells are for example tumor cells, cells invaded by a pathogen such as a virus, and autoimmune cells. Thus, in one embodiment, provided is an immunoglobulin according to any of the aforementioned embodiments wherein the MHC-peptide complex is specific for aberrant cells.

Thus the invention provides a method for eradicating aberrant cells, in particular tumor cells expressing on their surface a MHC-peptide complex comprising a peptide derived from MAGE comprising contacting said cell with at least one immune effector cell through specific interaction of a specific binding molecule for said MHC-peptide complex. According to the invention the immune effector cells are brought into close proximity of aberrant cells. It is an important aspect of the invention that the target on the tumor cell, the MAGE/MHC-I peptide complex, is tumor specific. Therefore the effector cells attracted to the target will typically only induce cell death in aberrant cells. There are several ways of bringing immune effector cells, in particular NK cells and T cells, in close proximity of the aberrant cells. Any such method that uses the MAGE/MHC-I peptide complex is in principle suitable for this invention. Preferred ones involve bispecific molecules.

Another preferred method is to provide effector cells, in particular T cells, with a specific binding molecule recognizing the MAGE/MHC-I peptide complex. Thus, the invention provides a binding molecule comprising a binding domain specifically recognizing a certain MHC-peptide complex exposed on the surface of an aberrant cell and a binding domain capable of attracting effector immune cells to this aberrant cell. As used herein, the term “specifically binds to a MHC-peptide complex” means that said molecule has the capability of specifically recognizing and binding a certain MHC-peptide complex, in the situation that a certain MHC-peptide complex is present in the vicinity of said binding molecule. Likewise, the term “capable of recruiting immune effector cells” means that the said molecule has the capability of specifically recognizing and binding antigens specific to immune effector cells when said immune effector cells are present in the vicinity of said specific binding molecule.

The term “specifically binds” means in accordance with this invention that the molecule is capable of specifically interacting with and/or binding to at least two amino acids of each of the target molecule as defined herein. Said term relates to the specificity of the molecule, i.e. to its ability to discriminate between the specific regions of the target molecule. The specific interaction of the antigen-interaction-site with its specific antigen may result in an initiation of a signal, e.g. due to the induction of a change of the conformation of the antigen, an oligomerization of the antigen, etc. Further, said binding may be exemplified by the specificity of a “key-lock-principle”. Thus, specific motifs in the amino acid sequence of the antigen-interaction-site and the antigen bind to each other as a result of their primary, secondary or tertiary structure as well as the result of secondary modifications of said structure. The specific interaction of the antigen-interaction-site with its specific antigen may result as well in a simple binding of said site to the antigen.

The term “binding molecule” as used in accordance with the present invention means that the bispecific construct does not or essentially does not cross-react with (poly)peptides of similar structures. Cross-reactivity of constructs under investigation may be tested, for example, by assessing binding of said constructs under conventional conditions (see, e.g., Harlow and Lane, Antibodies: A Laboratory Manual, Cold Spring Harbor Laboratory Press, 1988 and Using Antibodies: A Laboratory Manual, Cold Spring Harbor Laboratory Press, 1999) to the antigens of interest as well as to a number of more or less (structurally and/or functionally) closely related antigens. Only those constructs that bind to the antigens of interest but do not or do not essentially bind to any of the other antigens are considered specific for the antigen of interest.

If according to the invention a bispecific molecule is used it is clear to the skilled person that any format of a bispecific molecule as disclosed herein before (such as BiTEs, DARTs etc.) are suitable. Typically these formats will comprise a single polypeptide format or complexes of different polypeptide chains. These chains/polypeptides will typically comprise VH, VHH and/or VL.

Some formats of bispecific molecules, such as IgGs, include an Fc region. This is another binding moiety for immune effector cells. In formats where there is already an arm recognizing a target on the immune effector cell this moiety may be disabled through known means.

In a preferred embodiment a bispecific molecule comprises one arm specifically binding to a MHC-peptide complex comprising a peptide derived from MAGE associated with aberrant cells, and the other arm specifically recognizing a target associated with immune effector cells. Therefore the invention provides bispecific molecules according to the invention, wherein said bispecific molecule is a human IgG, preferentially human IgG1 wherein the Fc part does not activate the Fc receptor.

The invention includes IgG sequences of mouse, rat, rabbit, human and camelid origin. The immunoglobulin single variable domain includes fully human, humanized, sequence optimized or chimeric immunoglobulin sequences.

The advantage of targeting MAGE-A has been described in our early application US-2015-0056198 incorporated herein by reference. Briefly, MAGE-A expression is restricted to, apart from testis and placenta, aberrant cells. Placenta and testis do not express classical MHC, de facto MAGE-A/MHC-I peptide complexes are tumor specific targets. Because there are many possible combinations of MHC molecules and MAGE-A peptides it is possible to device alternating and/or combination therapies, which tackles the problem of tumor escape from therapy.

The term ‘immune effector cell’ or ‘effector cell’ as used herein refers to a cell within the natural repertoire of cells in the mammalian immune system which can be activated to affect the viability of a target cell. Immune effector cells include the following cell types: natural killer (NK) cells, T cells (including cytotoxic T cells), B cells, monocytes or macrophages, dendritic cells and neutrophilic granulocytes. Hence, said effector cell is preferably an NK cell, a T cell, a B cell, a monocyte, a macrophage, a dendritic cell or a neutrophilic granulocyte. According to the invention, recruitment of effector cells to aberrant cells means that immune effector cells are brought in close proximity to the aberrant target cells resulting in formation of immunological synapse and activation of said immune effector cells, such that the effector cells can kill (directly or indirectly by initiation of the killing process) the aberrant cells that they are recruited to. Activation of the immune effector cells causes one or more cellular responses such as proliferation, differentiation, cytokine secretion, cytotoxic effector molecule release (e.g. perforins and granzymes), cytotoxic activity, expression of activation markers and redirected target cell lysis.

Target antigens present on immune effector cells may include CD3, CD16, CD25, CD28, CD64, CD89, NKG2D and NKp46. The most preferred antigen on an immune effector cell is the CD3 epsilon chain.

T cells are an example of immune effector cells, that can be attracted by the said specific binding molecule to the aberrant cells. CD3 is a well described marker of T cells that is specifically recognized by antibodies described in the prior art. Furthermore, antibodies directed against human CD3 are generated by conventional methods known in the art. The VH and VL regions of said CD3 specific domain are derived from a CD3 specific antibody, such e.g. but not limited to, OKT-3 or TR-66. In accordance with this invention, said VH and VL regions are derived from antibodies/antibody derivatives and the like which are capable of specifically recognizing human CD3 epsilon in the context of other TCR subunits.

Methods of treating cancer with antibodies are well known in the art and typically include parental injection of efficacious amounts of antibodies which are typically determined by dose escalation studies.

An aspect of the invention relates to a bispecific molecule according to the invention for use in the treatment of cancer.

Another method of bringing together immune effector cells and aberrant cells is to provide immune effector cells with a cell surface associated molecule, typically a receptor. In this case, according to the invention, typically T cells are provided with a T cell receptor and/or a chimeric antigen receptor that specifically recognizes MAGE-A/MHC-I peptide complexes. Therefore the invention provides a method according to said invention wherein said specific binding molecule is a T cell receptor and/or chimeric antigen receptor. These T cells are made by introducing into said T cell nucleic acids encoding an α chain and a β chain or a chimeric antigen receptor.

The dosage of the specific binding molecules are established through animal studies, (cell-based) in vitro studies, and clinical studies in so-called rising-dose experiments. Typically, the doses of present day antibody are 3-15 mg/kg body weight, or 25-1000 mg per dose, present day BiTE 28 μg/day dose infused over 48 hours and 2×10⁶−2×10⁸ CAR-positive viable T cells per kg body weight of present day CAR-T cells.

For administration to subjects the specific binding molecule hereof must be formulated. Typically the specific binding molecules will be given intravenously. For formulation simply water (saline) for injection may suffice. For stability reasons more complex formulations may be necessary. The invention contemplates lyophilized compositions as well as liquid compositions, provided with the usual additives.

Binding molecules having the VH domains given in SEQ ID NO:1, SEQ ID NO:2 and SEQ ID NO:61 have been shown to have sufficient affinity and specificity to be used according to the invention. The VL domain of 4A6 antibody is represented by SEQ ID NO: 59, the VL domain of A09 antibody is represented by SEQ ID NO: 60 and the VL domain of murine 9A7 antibody is represented by SEQ ID NO: 62.

Many binding domains able to specifically bind to MHC-peptide complexes are apparent to people of skill in the art. Immediately apparent are binding domains derived from the immune system, such as TCR domains and immunoglobulin (Ig) domains. Preferably, the domains encompass 100 to 150 amino acid residues. Preferably, the binding domains used for the invention are or are similar to variable domains (VH or VL) of antibodies. A good source for such binding domains are phage display libraries. Whether the binding domain of choice is actually selected from a library physically or whether only the information (sequence) is used is of little relevance. It is part of the invention that the binding molecule according to the invention preferably encompasses two or more variable domains of antibodies (“multispecificity”), linked through peptide bonds with suitable linker sequences. The term “antibody” used in this invention includes intact molecules (whole IgG) as well as fragments thereof, such as: Fab, F(ab′)2, Fv, single chain Fv (“scFv”), disulphide-stabilized Fv (“dsFv”), or single domain molecules such as VH and VL that are capable of binding to MHC/peptide complexes.

Functional antibody fragments comprising whole or essentially whole variable regions of both heavy and light chains are defined as follows:
(i) Fv, defined as genetically engineered fragment consisting of the variable region of the light chain (VL) and the variable region of the heavy chain (VH) expressed as two chains;
(ii) Single chain Fv (“scFv”), a genetically engineered single chain molecule including the variable region of the light chain (VL) and the variable region of the heavy chain (VH), linked by a suitable polypeptide linker as a genetically fused single chain molecule;
(iii) Disulphide-stabilized Fv (“dsFv”), a genetically engineered antibody molecule including the variable region of the light chain (VL) and the variable region of the heavy chain (VH), linked by a genetically engineered disulphide bond;
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain (VH) which consists of the variable and CH1 domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′)2, a fragment of an antibody molecule containing a monovalent antigen-binding portion of an antibody molecule which can be obtained by treating whole antibody with the enzyme pepsin (i.e. a dimer of Fab′ fragments held together by two disulphide binds); and
(vii) Single domain antibodies or nanobodies are composed of a single VH or VL domains which exhibit sufficient activity to the antigen.

As stated before the binding domains selected according to the invention are preferably based on, or derived from an immunoglobulin domain. The immunoglobulins (Ig) are suitable for the specific and selective localization attraction of immune effector cells to targeted aberrant cells, leaving healthy cells essentially unaffected. Immunoglobulins comprise immunoglobulin binding domains, referred to as immunoglobulin variable domains, comprising immunoglobulin variable regions. Maturation of immunoglobulin variable regions results in variable domains adapted for specific binding to a target binding site.

According to the present invention the term “variable region” used in the context with Ig-derived antigen-interaction comprises fragments and derivatives of (poly)peptides which at least comprise one CDR derived from an antibody, antibody fragment or derivative thereof. It is envisaged by the invention, that said at least one CDR is preferably a CDR3, more preferably the CDR3 of the heavy chain of an antibody (CDR-H3). Because the anticipated predominant use of the binding molecule hereof is in therapeutic treatment regimens meant for the human body, the immunoglobulins variable regions preferably have an amino-acid sequence of human origin. Humanized immunoglobulin variable regions, with the precursor antibodies encompassing amino acid sequences originating from other species than human, are also part hereof. Also part hereof are chimeric molecules, comprising (parts of) an immunoglobulin variable region hereof originating from a species other than human. Methods for humanizing non-human antibodies are well known in the art.

The affinity of the specific binding molecule hereof for the two different target binding sites separately, preferably is designed such that Kon and Koff are very much skewed towards binding to both different binding sites simultaneously. Thus, in one embodiment hereof, the antibody according to any of the previous embodiments is a hetero-dimeric bi-specific immunoglobulin G or heavy-chain only antibody comprising two different but complementary heavy chains. The two different but complementary heavy chains may then be dimerized through their respective Fc regions. Upon applying preferred pairing biochemistry, hetero-dimers are preferentially formed over homo-dimers. For example, two different but complementary heavy chains are subject to forced pairing upon applying the “knobs-into-holes” CH3 domain engineering technology as described (Ridgway et al., Protein Engineering, 1996 (ref 14)). In a preferred embodiment hereof the two different immunoglobulin variable regions in the bi-specific immunoglobulins hereof specifically bind with one arm to an MHC-peptide complex preferentially associated with aberrant cells, and to antigen present on immune effector cells.

Although the invention contemplates many different combinations of MHC and antigenic peptides the most preferred is the combination of MHC-1 and an antigenic peptide from a tumor related antigen presented by MHC-1. Because of HLA restrictions, there are many combinations of MHC-1-peptide complexes as well as of MHC-2-peptide rules include size limits on peptides that can be presented in the context of MHC, restriction sites that need to be present for processing of the antigen in the cell, anchor sites that need to be present on the peptide to be presented, etc. The exact rules differ for the different HLA classes and for the different MHC classes. We have found that MAGE derived peptides are very suitable for presentation in an MHC context. An MHC-1 presentable antigenic peptide with the sequence Y-L-E-Y-R-Q-V-P-G in MAGE-A was identified, that is present in almost every MAGE-A variant (referred to as multiMAGE peptide or mMA) and that will be presented by one of the most prevalent MHC-1 alleles in the Caucasian population (namely HLA-A*0201). A second MAGE peptide that is presented by another MHC-1 allele (namely HLA-CW7) and that is present in many MAGE variants, like, for example, MAGE-A2, -A3, -A6 and -A12, is E-G-D-C-A-P-E-E-K. These two combinations of MHC-1 and MAGE peptides together could cover 80% of the Caucasian population. Another MAGE peptide that is presented by the same MHC-I allele as the multiMAGE peptide has a sequence F-L-W-G-P-R-A-L-V and is present in MAGE-A3 and MAGE-A12 proteins. A MAGE peptide that is presented by a different MHC-1 allele, namely HLA-A*2402, has a sequence I-M-P-K-T-G-F-L-1 and is present in MAGE-A1 and MAGE-A6. HLA-A*2402 is a highly prevalent allele in Asian population.

Thus, in one embodiment, provided is a list of MAGE-A derived peptides presented in context of HLA-A0201, HLA-A2402 and HLA-C0701.

The term “cancer” as used herein is defined as disease characterized by the rapid and uncontrolled growth of aberrant cells. Cancer cells can spread locally or through the bloodstream and lymphatic system to other parts of the body. An “aberrant cell” is defined as a cell that deviates from its healthy normal counterparts. Aberrant cells are, for example, tumor cells and autoimmune cells.

### Numbered Embodiments of the Invention

1. A bispecific molecule of which one arm comprises a first domain that specifically binds to a MHC-peptide complex comprising a peptide derived from MAGE expressed on the cell surface of aberrant cells, and the other arm comprises a second domain that specifically recognizes a target expressed on the cell surface of immune effector cells.
2. Bispecific molecule according to embodiment 1, wherein the first domain comprises a VH, VHH or VL.
3. Bispecific molecule according to embodiment 1 or 2, wherein said second domain comprises a VH, VHH or VL.
4. Bispecific molecule according to any one of the previous embodiments 1-3, wherein the first domain is a VHH and/or the second domain is a VHH, preferably both are VHH.
5. Bispecific molecule according to any one of the previous embodiments 1-4, wherein the molecule is a bispecific antibody.
6. Bispecific molecule according to any one of the previous embodiments 1-5, which is in a BiTE format.
7. Bispecific molecule according to any one of the previous embodiments 1-6, wherein the first domain binds specifically to a MHC/peptide complex comprising a peptide derived from MAGE-A.
8. Bispecific molecule according to any one of the previous embodiments 1-7, wherein the first domain is a VHH domain according to SEQ ID NO: 47; SEQ ID NO: 48; SEQ ID NO:49; SEQ ID NO: 50; SEQ ID NO: 51; SEQ ID NO: 52; SEQ ID NO: 53; SEQ ID NO: 54; SEQ ID NO: 55; or SEQ ID NO: 56.
9. Bispecific molecule according to any one of the previous embodiments 1-8, wherein the target recognized by the second domain is expressed on the cell surface of a T cell or NK-cell.
10. Bispecific molecule according to any one of the previous embodiments 1-9, wherein the target recognized by the second domain is CD3, preferably CD3 on a T-cell or NK-cell.
11. Bispecific molecule according to any one of the previous embodiments 1-10, wherein second domain is a VHH domain according to SEQ ID NO: 57.
12. Bispecific molecule according to any one of the previous embodiments 1-11, wherein the first domain is a VHH domain according to SEQ ID NO: 47; SEQ ID NO: 48; SEQ ID NO:49; SEQ ID NO: 50; SEQ ID NO: 51; SEQ ID NO: 52; SEQ ID NO: 53; SEQ ID NO: 54; SEQ ID NO: 55; or SEQ ID NO: 56; and the second domain is a VHH domain according to SEQ ID NO: 57.
13. A pharmaceutical composition comprising a bispecific antibody according to any one of the previous embodiments 1-12 and suitable diluents and/or excipients.
14. A pharmaceutical composition according to embodiment 13, comprising a further bispecific antibody according to any one of the embodiments 1-12, having at least one different specificity compared to the first antibody.
15. A pharmaceutical composition according to embodiment 13, comprising a second bispecific antibody according to any one of the embodiments 1-11 having at least one different specificity compared to the first antibody.
16. Bispecific molecule according to any one of the embodiments 1-12, or a pharmaceutical composition thereof according to any one of the embodiments 13-15 for use in the treatment of cancer.
17. Bispecific molecule according to any one of the embodiments 1-12, or a pharmaceutical composition thereof according to embodiment 13-15 for use in the treatment of lung cancer, in particular for use in the treatment of non-small-cell lung carcinoma.
18. A method for eradicating tumor cells expressing on their surface a MHC-peptide complex comprising a peptide derived from MAGE comprising contacting said cell with at least one immune effector cell through specific interaction of a specific binding molecule for said MHC-peptide complex, wherein said specific binding molecule is a bispecific molecule according to any one of the embodiments 1-11.
19. The method according to embodiment 18, wherein said cell is contacted with at least one further bispecific molecule according to any one of the embodiments 1-11, said further bispecific molecule having at least one different specificity compared to the first bispecific molecule.
20. The method according to embodiment 18, wherein the VHH domain for binding to an MHC/MAGE peptide complex is according to SEQ ID NO: 47; SEQ ID NO: 48; SEQ ID NO:49; SEQ ID NO: 50; SEQ ID NO: 51; SEQ ID NO: 52; SEQ ID NO: 53; SEQ ID NO: 54; SEQ ID NO: 55; or SEQ ID NO: 56.
21. The method according to any one of the previous embodiments 18-20, wherein a VHH domain for binding to CD3 is according to SEQ ID NO: 57.
22. The method according to any one of the previous embodiments 18-21, wherein the binding molecule has a BiTE format.
23. The method according to any one of the previous embodiments 18-22 for the treatment of cancer, in particular lung cancer, more in particular non-small-cell lung cancer.

### EXAMPLES

The invention is exemplified by the following non-limiting Examples.

### Example 1

Target binding sites suitable for specific and selective targeting of aberrant cells by specific binding molecules of the invention are MAGE-derived antigen peptides complexed with MHC molecules. Examples of T-cell epitopes of the MAGE-A protein, complexed with indicated HLA molecules, are provided below. Any combination of an HLA molecule complexed with a MAGE-derived T-cell epitope provides a specific target on aberrant cells for specific binding molecules hereof. Examples of suitable target MAGE-derived epitopes are peptides: FRAVITKKV, KVSARVRFF, FAHPRKLLM, SVFAHPRKL, LRKYRAKEL, FREALSNKV, VYGEPRKLL, SVYWKLRKL, VRFLLRKYQ, FYGEPRKLL, RAPKRQRCM, LRKYRVKGL, SVFAHPRKL, VRIGHLYIL, FAHPRKLLT presented via HLA-C*0701; IMPKTGFLI, VSARVRFFF, NYKHCFPEI, EYLQLVFGI, VMPKTGLLI, IMPKAGLLI, NWQYFFPVI, VVGNWQYFF, SYPPLHEWV, SYVKVLHHM, IFPKTGLLI, NYKRCFPVI, IMPKTGFLI, NWQYFFPVI, VVGNWQYFF, SYVKVLHHM, RFLLRKYQI, VYYTLWSQF, NYKRYFPVI, VYVGKEHMF, CYPSLYEEV, SMPKAALLI, SSISVYYTL, SYEKVINYL, CYPLIPSTP, LYDGMEHLI, LWGPITQIF, VYAGREHFL, YAGREHFLF, EYLQLVFGI, SYVKVLHHL presented via HLA-A*2402; KVLEYVIKV, FLIIVLVMI, FLWGPRALA, YVIKVSARV, LVLGTLEEV, CILESLFRA, IMPKTGFLI, KVADLVGFL, YVLVTCLGL, KASESLQLV, KMVELVHFL, KIWEELSML, FLWGPRALI, KASEYLQLV, YILVTCLGL, GLLIIVLAI, LQLVFGIEV, HLYILVTCL, QLVFGIEVV, LLIIVLAII, GLVGAQAPA, FLWGPRALV, KVAELVHFL, YIFATCLGL, KIWEELSVL, ALSRKVAEL, GLLIIVLAI, FQAALSRKV, HLYIFATCL, LLIIVLAII, GLVGAQAPA, KVLHHMVKI, GNWQYFFPV, KVLEHWRV, ALLEEEEGV, FLWGPRALA, KVDELAHFL, ALSNKVDEL, AVSSSSPLV, YTLVTCLGL, LLIIVLGTI, LVPGTLEEV, YIFATCLGL, FLWGPRALI, KIWEELSVL, FLIIILAII, KVAKLVHFL, IMPKTGFLI, FQAALSRKV, KASDSLQLV, GLVGAQAPA, KVLHHMVKI, GNWQYFFPV, GLMDVQIPT, LIMGTLEEV, ALDEKVAEL, KVLEHWRV, FLWGPRALA, LMDVQIPTA, YILVTCLGL, KVAELVRFL, AIWEALSVM, RQAPGSDPV, GLLIIVLGM, FMFQEALKL, KVAELVHFL, FLWGSKAHA, ALLIIVLGV, KVINYLVML, ALSVMGVYV, YILVTALGL, VLGEEQEGV, VMLNAREPI, VIWEALSVM, GLMGAQEPT, SMLGDGHSM, SMPKAALLI, SLLKFLAKV, GLYDGMEHL, ILILSIIFI, MLLVFGIDV, FLWGPRAHA, GMLSDVQSM, KMSLLKFLA, FVLVTSLGL, KVTDLVQFL, VIWEALNMM, NMMGLYDGM, QIACSSPSV, ILILILSII, GILILILSI, GLEGAQAPL, AMASASSSA, KIIDLVHLL, KVLEYIANA, VLWGPITQI, GLLIIVLGV, VMWEVLSIM, FLFGEPKRL, ILHDKIIDL, FLWGPRAHA, AMDAIFGSL, YVLVTSLNL, HLLLRKYRV, GTLEELPAA, GLGCSPASI, GLITKAEML, MQLLFGIDV, KMAELVHFL, FLWGPRALV, KIWEELSVL, KASEYLQLV, ALSRKMAEL, YILVTCLGL, GLLGDNQIV, GLLIIVLAI, LQLVFGIEV, KVLHHLLKI, HLYILVTCL, QLVFGIEVV, LLIIVLAII, RIGHLYILV, GLVGAQAPA presented via HLA-A*0201.

A good source for selecting binding sites suitable for specific and selective targeting of aberrant cells hereof, is the NetMHC (on the WorldWideWeb at cbs.dtu.dk/services/NetMHC). The portal constitutes a prediction tool of peptide-MHC class I binding, upon uploading amino-acid sequence of antigen of interest in context of MHC molecules comprising the indicated class of HLA.

### Example 2

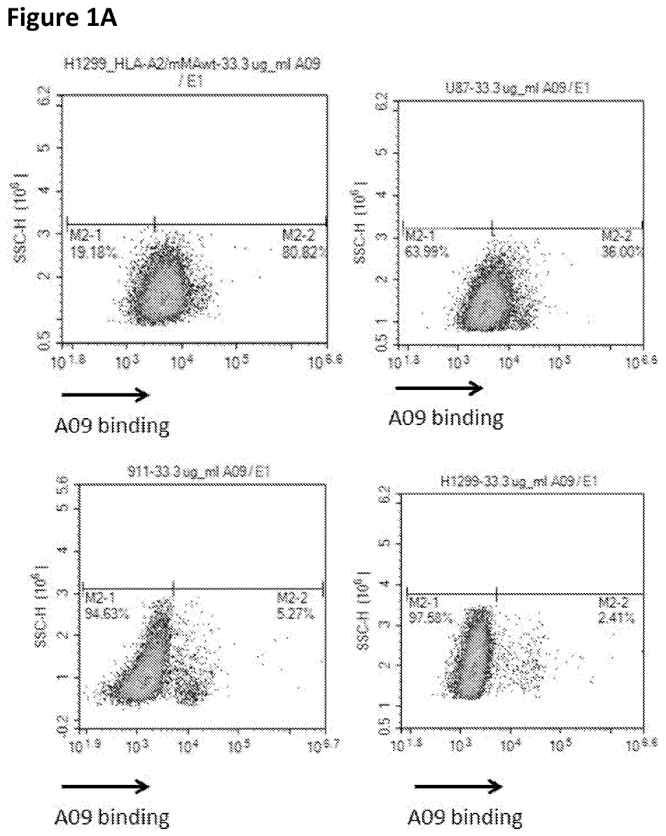

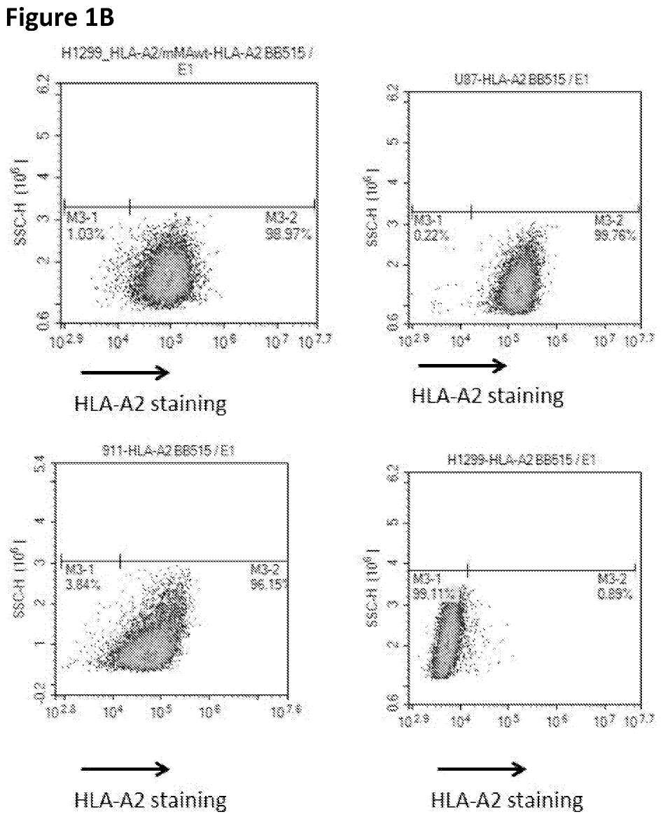

A09 IgG specifically binds human aberrant cells presenting mMA peptide via HLA-A2 In order to confirm specificity of A09 IgG, the molecule was incubated with a panel of cell lines differing in their HLA-A2 and MAGE expression. Employed cell lines include non small cell lung carcinoma H1299 (HLA-A2−, MAGE+), non small cell lung carcinoma H1299_A2/mMA cells stably transfected with an expression construct of HLA-A2/mMA (HLA-A2+, MAGE+), glioblastoma cells U87 (HLA-A2+, MAGE+) and embryonic retinoblasts 911 (HLA-A2+, MAGE−). Briefly, the cells were spun down for 4 min at 450×g at 4° C. The supernatant was gently removed and the cell pellet resuspended in 100 μl of PBS+0.1% BSA per sample. Cells were transferred to the designated wells of a 96-well plate (100 μl/well) and spun down for 4 min at 450×g at 4° C. The supernatant was gently removed. The tested antibody in PBS+0.1% BSA was added to the cell pellet (20 μl/sample). The plate was shortly vortexed, in a gentle manner, to resuspend the cell pellet. Cells were incubated for 30 min at 2-8° C., upon which 200 μl of ice-cold PBS+0.1% BSA were added per well. Cells were washed by spinning down for 4 min at 450×g at 4° C. The supernatant was gently removed. Washing step was repeated. The primary detection antibody was diluted in PBS+0.1% BSA and added to the cell pellet (20 μl/sample). Samples were incubate for 30 min at 2-8° C. with goat anti human H+L IgG Alexa647 or mouse anti human HLA A2 BB515. At the end of the incubation cells were washed twice as described before. Cells were fixed by resuspending the cell pellet in 200 μl of 1% PFA per sample at RT. The fluorescent signal was measured using Flow Cytometer. As shown in flow cytometric dot plots of FIG. 1A the A09 antibody specifically recognized the multi MAGE peptide in complex with HLA-A*0201. The expression of HLA-A*0201 by H1299_A2/mMA cells, U87 cells and 911 cells was confirmed as shown in FIG. 1B.

### Example 3

3.1 Mice Immunization and Generation of Hybridoma Producing Murine Antibodies with Desired Specificity to Respective HLA/MAGE-Derived Peptide Complex

BALB/C mice (n=4) were subjected to 3 consecutive, subcutaneous immunizations with HLA/MAGE_{1, 6}-peptide derived complexes as antigen (namely HLA-A*2402/IMPKTGFLI). Animals were immunized with 25 μg of antigen on days 1, 21 and 36. First immunization was performed with antigen using complete Freund's adjuvant (mixed with immunogen just before immunization), whereas all following immunizations were performed with antigen using incomplete Freund's adjuvant (mixed with immunogen just before immunization). Three to four days before isolation of mice spleen a boost immunization (intraperitoneal) with Freund's incomplete adjuvant containing the antigen was performed. In course of immunization pre-immune and post-immune serum samples were collected to confirm onset of immune response directed to used antigen. Based on the immune response profile, best responding animals were determined and chosen for subsequent splenocytes isolation. Splenocytes isolated from these animals were fused with myeloma cells Sp2/OAg 14 performed by using Polyethylene glycol (PEG). The hybridomas were plated out and grown in HA selective medium (Hypoxanthin-Azaserin). Binding of mouse immunoglobulins produced by the hybridomas was assessed in ELISA in which biotinylated HLA-A*2402/IMPKTGFLI complexes were coated to streptavidin plates. Hybridomas that survived culturing in HAT selective medium and showed specific binding to immunogen as assessed in ELISA, were cloned by serial dilution. For each clone, a master bank of 5 vials (10e6 cells/vial) was generated. A hybridoma producing a murine antibody specifically binding HLA-A*2402/IMPKTGFLI complexes was identified. The amino-acid sequences of the VH and VL of 9A7 immunoglobulin were identified (SEQ ID NO: 61 and SEQ ID NO: 62, respectively).

3.2 9A7 Hybridoma Produces Murine Antibody Specifically Binding Recombinant HLA-A*2402/IMPKTGFLI Complexes

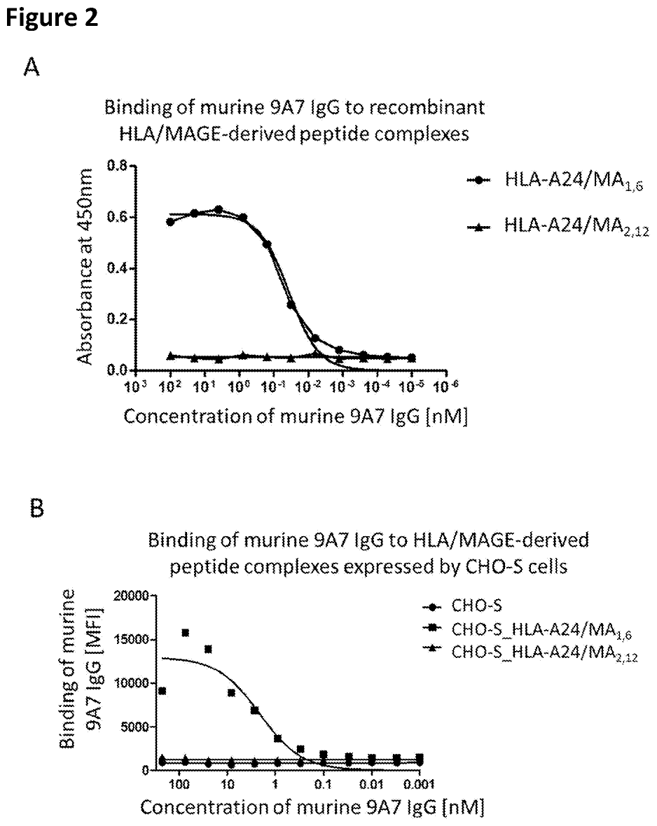

Binding characteristics of the 9A7 murine IgG obtained via hybridoma approach were assessed in ELISA on biotinylated HLA/MAGE-derived peptide complexes. Streptavidin coated 96-wells plates were pre-washed 3 times with PBS containing 0.05% Tween (0.05% PBST). Biotinylated HLA/MAGE-derived peptide complexes (annotated in the Figure as HLA-A*2402/MA_{2, 12} and HLA-A*2402/MA_{1, 6}) were coated at 0.5 μg/ml. HLA-A*2402/A_{2, 12} complexes are formed by HLA-A*2402 molecules and MAGE-A derived peptide, EYLQLVFGI, which can be derived from both MAGE-A2 and MAGE-A12. HLA-A*2402/A_{1, 6} complexes are formed by HLA-A*2402 molecules and MAGE-A derived peptide, IMPKTGFLI, which can be derived from both MAGE-A1 and MAGE-A6. The plate was incubated for 1 hour while shaking (120 rpm) at room temperature. After incubation the wells were washed 4 times with 0.05% PBST. The plate was blocked with 120 μl/well of 0.05% PBST containing 2% milk powder (PBSTM) for 1 hour while shaking at 120 rpm at room temperature. A dilution series of 9A7 murine antibody, starting at 100 nM concentration, was incubated with coated HLA/MAGE-derived peptide complexes in streptavidin coated plates. The incubation was conducted at room temperature for 1 hour while shaking at 120 rpm. Subsequently, the wells were washed 4 times with 0.05% PBST. For detection of bound 9A7 murine IgG the goat anti-mouse HRP Antibody (Santa Cruz, cat #sc-516102-cm) was used. The coating of the HLA/MAGE-derived peptide complexes to the plate was monitored with mouse anti-ABC W6/32 antibody and goat anti-mouse HRP (Thermo Scientific cat #MA1-70111), used respectively in concentrations of 0.4 μg/ml and 1 μg/ml. The plates were incubated for 1 hour while shaking (120 rpm) at room temperature prior to being washed 3 times with 0.05% PBST and 2 times with PBS. To develop the signal corresponding to bound 9A7 murine IgG, 50 μl Turbo TMB substrate (Sigma-Aldrich cat. no. SD247625) was added. The reaction was stopped by addition of 2M H₂SO₄. Absorbance measurement was performed at 450 nm on the iMark plate reader. Purified mouse 9A7 IgG bound specifically to the HLA-A*2402/IMPKTGFLI complexes with an IC50 of 0.037 nM (FIG. 2A) as determined by GraphPad Prism. No binding was detected to control complexes, namely HLA-A*2402/MA_{2, 12} (in which MAGE-A derived peptide, EYLQLVFGI, presented by HLA-A*2402 can be derived from both MAGE-A2 and MAGE-A12).

3.3 9A7 Hybridoma Produces Murine Antibody Specifically Binding HLA-A*2402/IMPKTGFLI Complexes Presented on Cell Surface

To determine the binding characteristics of 9A7 towards HLA/MAGE-derived peptide complexes in cellular setting a panel of CHO-S stable transfectants was used. The CHO-S cells were stably expressing either HLA-A*2402/IMPKTGFLI (referred to as CHO-S_HLA-A24/MA_{1, 6}) or HLA-A*2402/EYLQLVFGI (referred to as CHO-S_HLA-A24/MA_{2, 12}). Non transfected CHO-S cells were used as a control. For the staining 200′000 cells were suspended in 100 μl PBS containing 0.1% BSA. Murine 9A7 IgG was added in a 1:3 dilution series (concentration range used started at 222 nM). After 30 minutes incubation at 40° C. the cells were washed two times with 200 μl PBS containing 0.1% BSA. After the last wash step, 0.04 μg goat anti-mouse IgG-PE (BD Biosciences; cat #550589) was added for a 30 minutes incubation at 4° C. After the incubation, cells were washed again two times with 200 μl PBS containing 0.1% BSA. Thereafter the cells were fixed with 1% PFA and analysed using ACEA Novocyte 2000 (Bioscience Inc.) with a two-laser configuration (488 nm and 640 nm). Analysis showed that murine 9A7 IgG displays desired specificity to cells presenting MAGE-A1-derived and MAGE-A6-derived IMPKTGFLI peptide in context of HLA-A*2402. No binding was detected to either non transfected CHO-S cells (devoid of HLA-A*2402) or CHO-S transfected with a control MAGE-A derived peptide i.e. EYLQLVFGI derived from MAGE-A2 and MAGE-A12 (CHO-S_HLA-A24/MA_{2, 12}). This result confirms specificity of murine 9A7 IgG (FIG. 2B).

### Example 4

Chimerization of the Mouse 9A7 Antibody

It is known that the use of mouse antibodies for therapeutic purposes results in inconsistent treatment effectiveness. Many patients who were administered with therapeutic antibodies of mouse origin develop a new set of antibodies specifically recognizing the mouse antibodies. Such development of human anti-murine antibodies (HAMA) leads to decrease of the treatment's efficacy or diminishes its potency over time. To reduce the risk of occurrence of HAMA, murine 9A7 was genetically engineered into a chimeric antibody. The chimeric antibody was constructed by fusing the murine variable domains (VH and VL sequences) of 9A7 antibody with the constant domains of human IgG1. Such antibody is referred to as chimeric 9A7 antibody or chimeric 9A7 IgG.

### Example 5

Flow Cytometric Alanine Scanning Assay Determined Fine Specificity of 4A6 IgG, A09 IgG and Chimeric 9A7 IgG and Confirmed Capability of Said Antibodies to Bind Complexes of Multi-MAGE Derived Peptides Restricted by Either HLA-A*0201 or HLA-A*2402, Respectively.

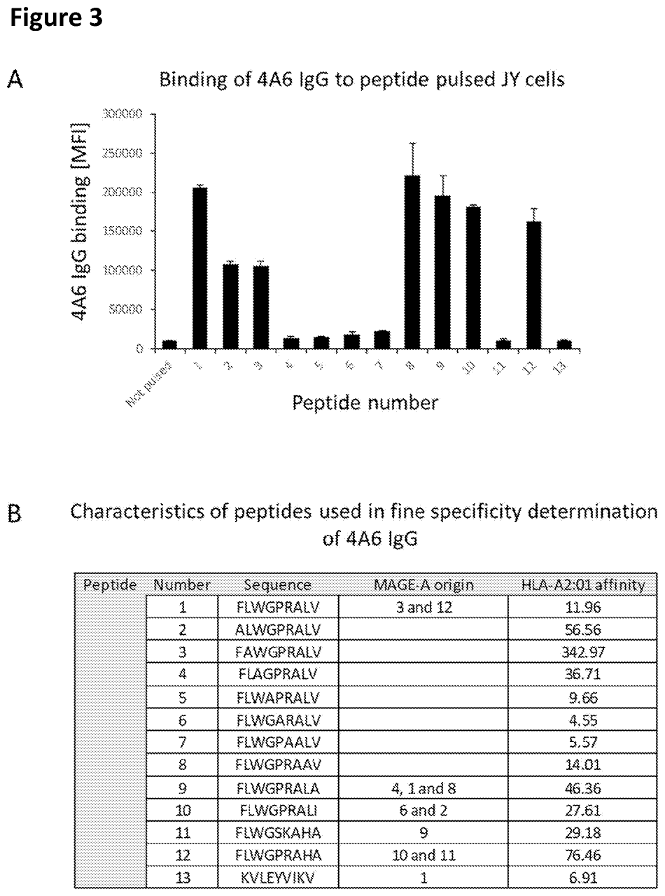

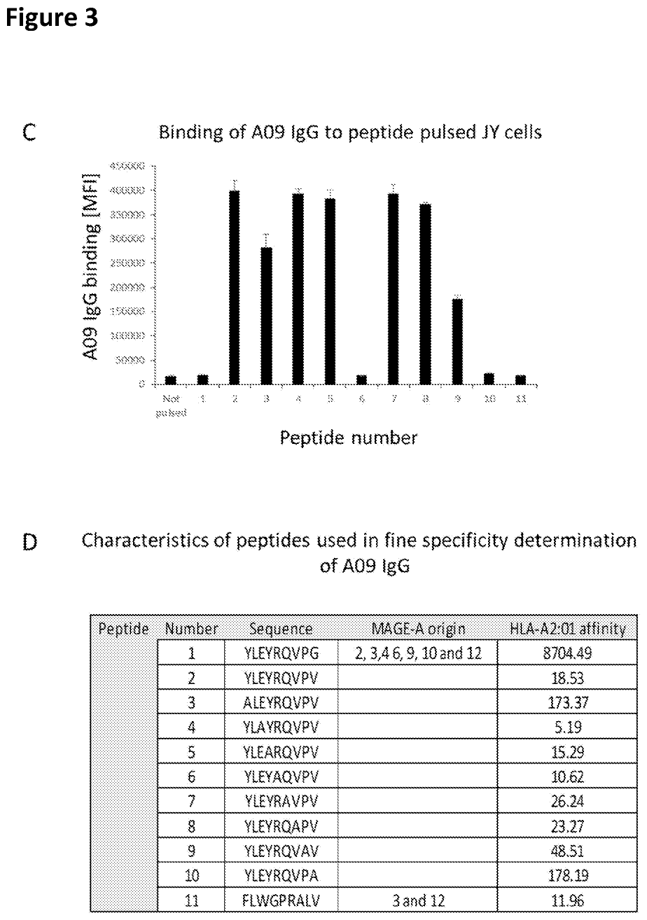

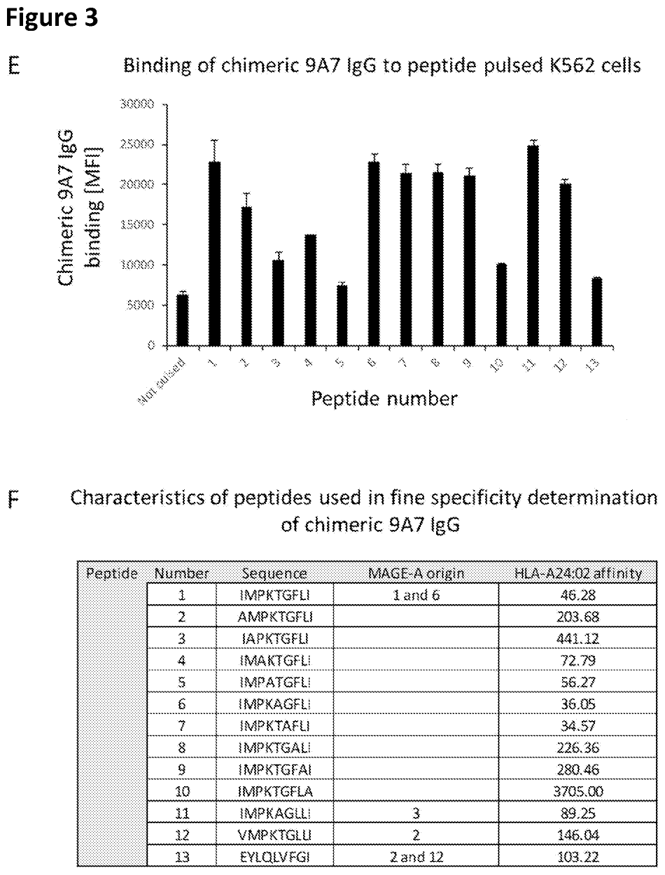

IgG fine specificity was determined in an alanine scanning approach in which peptides were pulsed onto HLA-A*0201 (HLA-A2) positive JY cells or HLA-A*2402 (HLA-A24) positive K562 cells. Briefly, 200′000 JY cells were incubated (pulsed) overnight under serum free conditions with 100 μg/ml peptide variants. The amino acids of the used peptides were sequentially substituted for an alanine (information regarding sequences of peptides is summarized FIG. 3B). Next day, pulsed JY cells were incubated for 1 hour with constant concentration of tested IgG (5 mg/ml. Bound antibody was detected using following detection antibody: anti-Human IgG-Alexa647 (Thermo Fisher Scientific, cat.no. A21445) Upon fixation with 1% PFA, samples were analysed using ACEA Novocyte 2000 (Bioscience Inc.) The flow cytometric results demonstrated that in case of 4A6 antibody the change of either of the first two amino acids of the FLWGPRALV to alanine affects 4A6 IgG binding. Replacement of either of amino acids in positions 3 till 6 by alanine resulted in complete lack of 4A6 IgG binding (FIG. 3A). Interestingly, the WGPR amino acids present in the core of the FLWGPRALV peptide derived from MAGE-A3 and MAGE-A12 (and required for binding of 4A6 IgG to HLA-A*0201/FLWGPRALV complexes) are conserved throughout all MAGE-A family members with exception of MAGE-A9. 4A6 IgG was shown to bind to JY cells pulsed with FLWGPRALI and FLWGPRAHA, which can be derived from MAGE-A2 and MAGE-A6, and MAGE-A10 and MAGE-A11, respectively. According to NetMHC these peptides have a high affinity towards HLA-A2 (below 37 nM) rendering them likely to be loaded onto HLA-A2 and presented on the cell surface of a malignant cell once produced by proteasome.

In the peptide pulsing experiment designed to determine key amino acid positions in the MAGE-derived peptide affecting the interaction of A09 IgG with HLA-A2/YLEYRQVPG complex, alanine substitutions were made in the YLEYRQVPV peptide. The substitution of glycine by valine in position 9 resulted in increased peptide affinity towards HLA-A2 and led to detection of A09 IgG binding to cells pulsed with YLEYRQVPV (referred to as mMA_{GV}). Flow cytometric analysis of A09 IgG binding to JY cells pulsed with alanine variants of YLEYRQVPV showed the importance of the proline at position 7 and the essential role of arginine at position 4 (FIG. 3C). Information regarding sequences of peptides used in this assessment is summarized FIG. 3D.

Peptide pulsing experiments aiming at determination of key peptide positions relevant for binding of chimeric 9A7 IgG to complexes restricted by HLA-A24 was performed using K562 cells. Information regarding sequences of peptides used in this assessment is summarized FIG. 3F. Flow cytometric analysis of chimeric 9A7 binding to pulsed cells demonstrated importance of several amino acids. The alanine substitution of isoleucine at position 1, methionine at position 2, proline at position 3 and lysine at position 4 as well as isoleucine at position 9 resulted in decreased binding of chimeric 9A7 antibody (FIG. 3E). According to NetMHC, of the important amino acids the methionine and isoleucine (position 2 and 9, respectively) are considered to be the ones determining the affinity of the peptide to HLA-A2402. The MPK amino acids found to strongly affect the binding of chimeric 9A7 IgG to HLA-A24/MAGE-derived peptide complex were also found to be present in MAGE-derived peptides originating from MAGE-A3 (IMPKAGLLI) and MAGE-A2 (VMPKTGLLI). The flow cytometric analysis of chimeric 9A7 IgG binding to cells pulsed with these peptides revealed comparable chimeric 9A7 IgG binding as obtained in case of cells pulsed with IMPKTGFLI peptide (FIG. 3E).

### Example 6

Generation of T Cells Specifically Recognizing MAGE-A Peptide Presented in Context of HLA-A0201

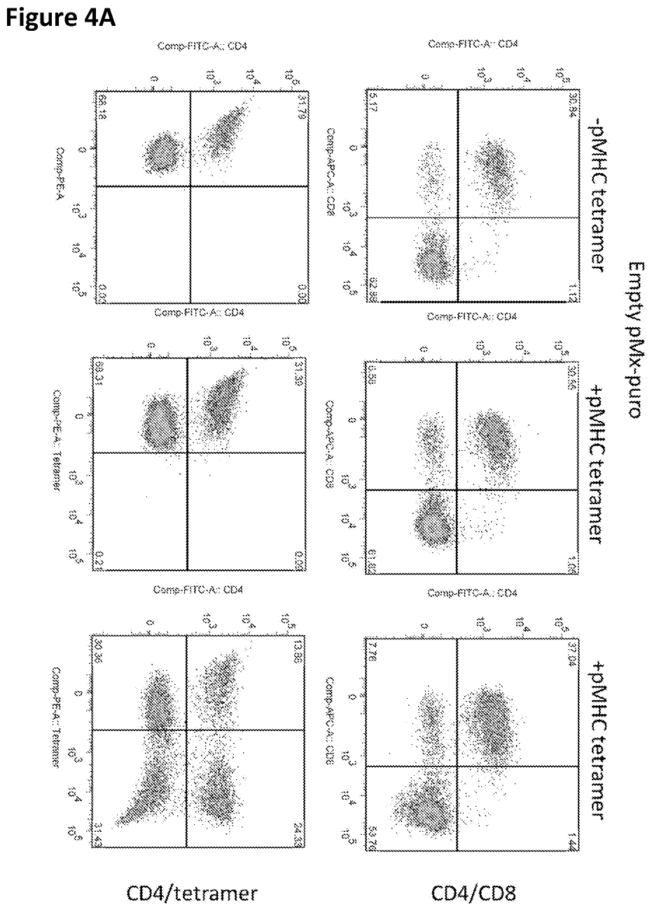

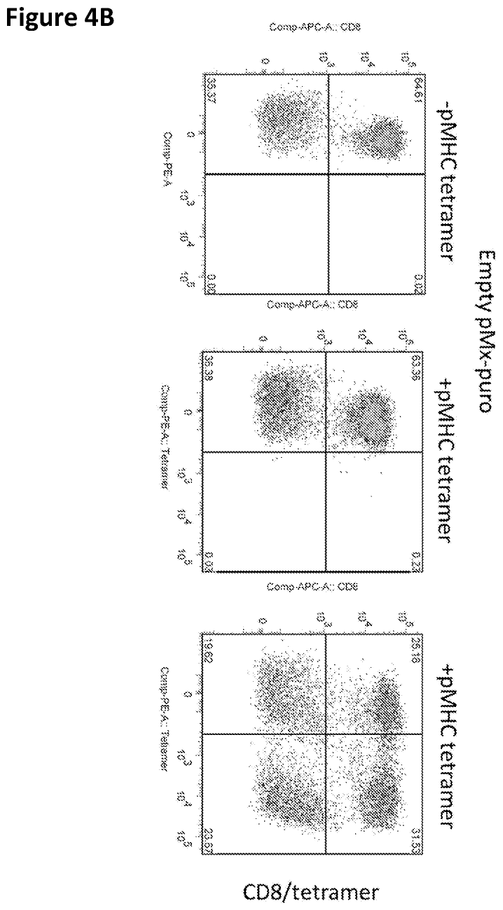

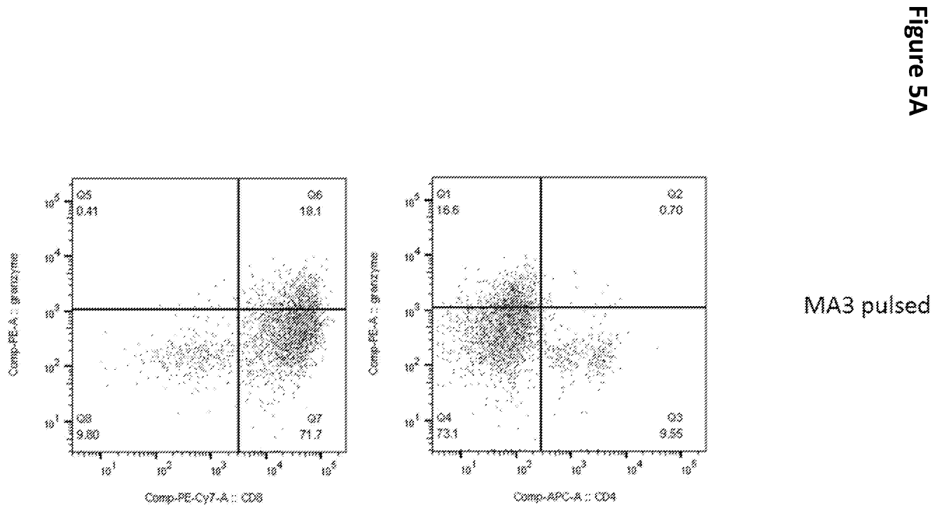

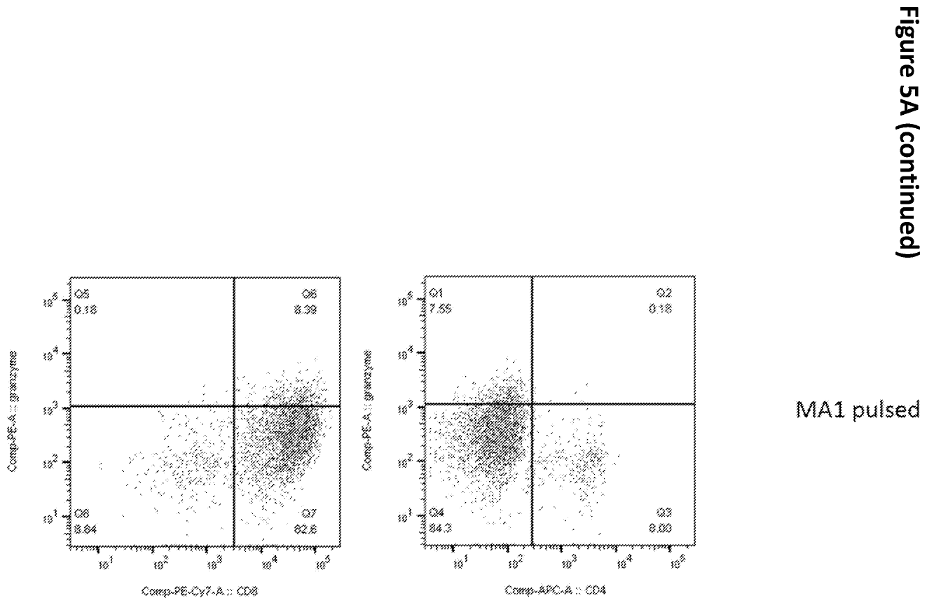

pMx-puro RTV014 vector and vector encoding scFv 4A6 CAR sequence were digested with BamHI and NotI. Digestion products were extracted from 1% agarose gel and purified using a DNA purification kit. The scFv 4A6 CAR purified fragments were ligated at 4° C. O/N with the purified pMx-puro RTV014 using the T4 ligase. Heat shock transformation of competent XL-I blue bacteria followed. Selection of transformed clones was based on ampicillin resistance (100 μg/ml). Plating of bacteria was performed on LB agar plates. Colonies were screened using restriction analysis. DNA was isolated using the Mini-prep DNA Isolation kit. Positive clones were grown in 100 ml LB+100 μg/ml ampicillin cultures. Phoenix Ampho cells were seeded at 1.2*10\hat{ }6 cells per 10 cm dish in DMEM (supplemented with 10% (V/V) fetal calf serum, 200 mM glutamine, 100 U penicillin, 100 μg/ml streptomycin), one day before transfection. Medium was refreshed 4 hrs prior transfection. 800 μl serum free DMEM were mixed with 35 μl of Fugene 6 reagent and incubated at RT for 5 min. 10 μg DNA (scFv 4A6 CAR pMx-puro RTV014) and 5 μg of each of the helper plasmids pHit60 and pColt-Galv were added to the mix. After incubating at RT for 15 min, the mix was added to the Phoenix Ampho cells. On the same day, PBMCs were thawed and seeded at a density of 2*10\hat{ }6 cells/well in a 24 well plate in 2 ml huRPMI containing 30 ng/ml of OKT-3 antibody and 600 U/ml IL-2. OKT-3 antibody was added to favor the proliferation of T cells in the PBMCs mixture. 24 hrs later, the medium of the transfected Phoenix Ampho cells was replaced with huRPMI. The day after, the transduction was initiated. The viral supernatant was collected by centrifugation at 2000 rpm at 32° C. for 10 min. T cells were also collected by centrifugation at 1500 rpm at RT for 5 min. 2*10\hat{ }6 T cells were resuspended in 0.5 ml of viral supernatant with 5 μg/ml polybrene in a 24-well plate. Plates were spun at 2000 rpm for 90 min. T cells were cultured at 37° C. O/N. The next day T cells were stimulated non-specifically with human CD3/CD28 beads. For specific stimulation of T cells peptide pulsed K562-HLA-A2-CD80 and 600 U/ml IL-2 were used. K562-HLA-A2-CD80 were pulsed with 10 μg peptide at 37° C. for 2 h. Cells were then irradiated at 10,000 rad. 0.3*10\hat{ }6 of pulsed and irradiated K562-HLA-A2-CD80 cells were added to 0.5*10\hat{ }6 T cells in a final volume of 2 ml huRPMI/well in a 24 well plate. Detection of scFv 4A6 CAR was performed by flow cytometric staining using tetramers of HLA-A2-MA3 (FLWGPRALV)-PE (0.5 μl/sample). The tetramers were produced by mixing biotinylated HLA-A2-MA3 (FLWGPRALV) complexes with PE streptavidin at a molar ratio 5:1. Samples were incubated at 4° C., in the dark for 30 minutes. Flow cytometric staining shown in FIGS. 4A and 4B confirmed presence of 4A6 CAR-T cells.

### Example 7

Apoptosis Induction of Target Expressing Cells Upon Facilitating T Cells with Specific Binding Molecule of the Disclosure

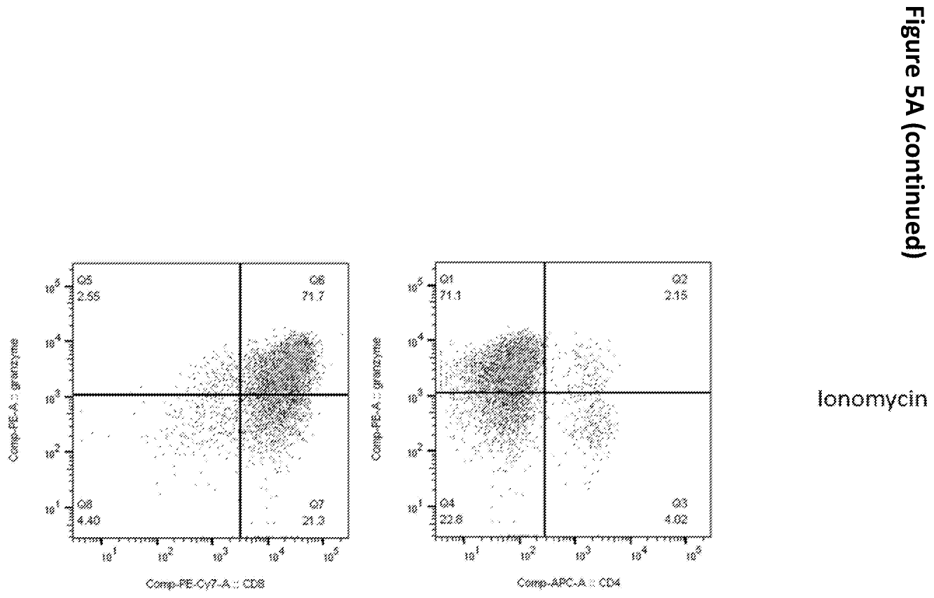

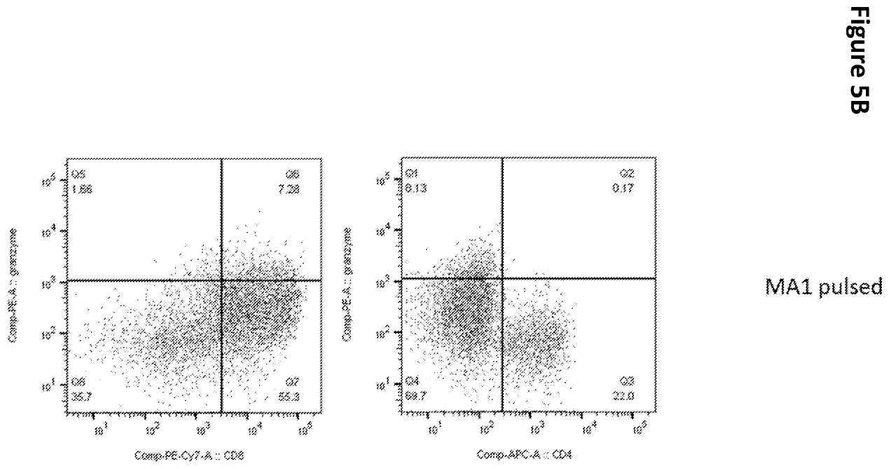

CD4 and CD8 T cells can cause target cell apoptosis through the perforin-granzyme pathway. These components are included in cytoplasmic granules of the effector cells. Upon CD3/TCR activation of T cells the granules are secreted and granzymes and perforin act synergistically to induce apoptosis. To determine whether or not the T cells expressing the MAGE-A specific CAR of the disclosure lead to T cell activation and apoptosis a flow cytometric assay was performed. scFv 4A6 CAR T cells were co-incubated for five hours with T2 cells pulsed either with the relevant MA3 peptide or with the irrelevant MA1 peptide. Both peptides show high affinity to HLA-A2 based on Net-MHC prediction. The calcium ionophore, ionomycin, a general T cell activator was used as a positive control. T cells transduced with pMx-puro RTV014 (not expressing scFv CAR) were used as a negative control. As expected, the positive control, ionomycin, led to high granzyme B production, independently of the type of transduced T cells (bottom panel of FIGS. 5A and 5B). Specific activation of scFv 4A6 CAR T cells by T2-MA3 pulsed cells was recorded (FIG. 5B, middle panel, left dot plot). The activated T cells belong mainly to the CD8 positive fraction, even though there is some minor reactivity by the CD4 subtype.

### Example 8

Purification and Specificity of Bispecific Molecules of the Disclosure Targeting HLA-A2/MAGE-A Derived Peptides Complexes and CD3.

8.1 Binding of the Bispecific Molecule of the Disclosure to HLA-A2/MAGE-A Derived Peptide Complexes

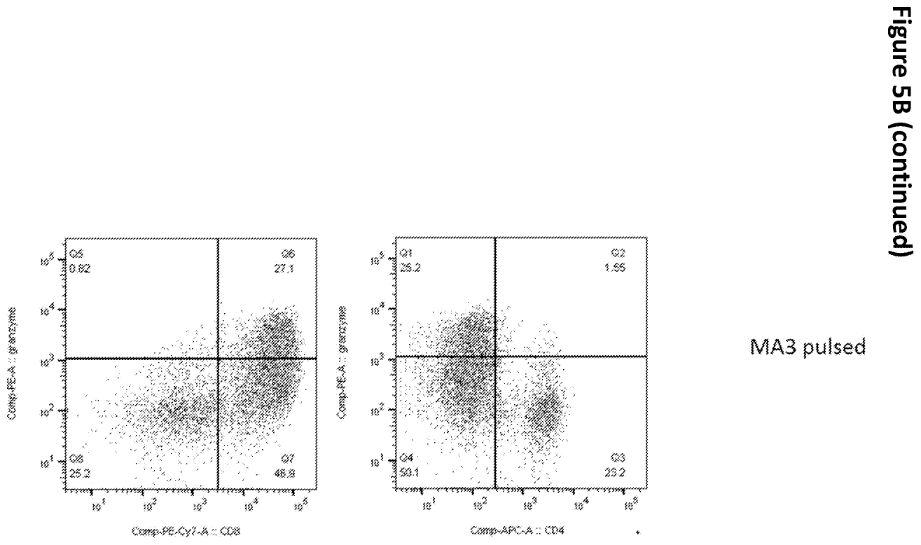

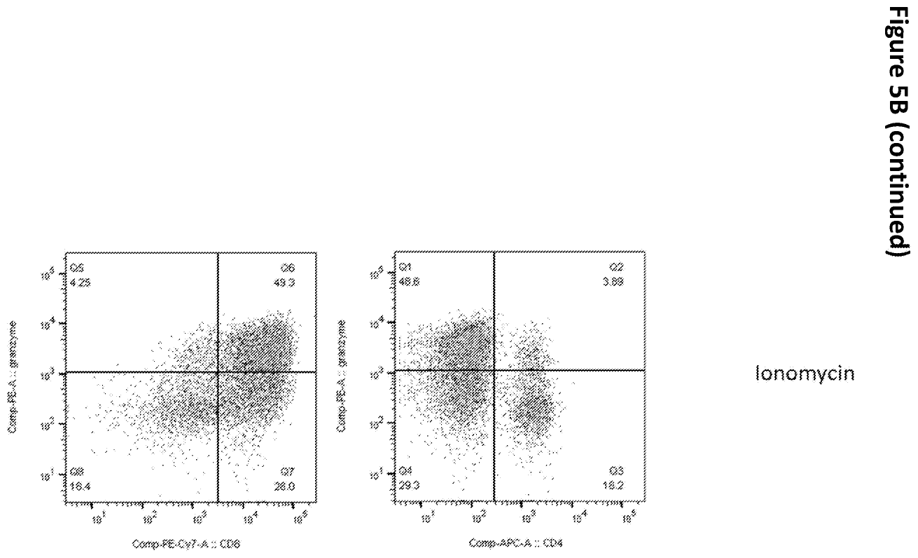

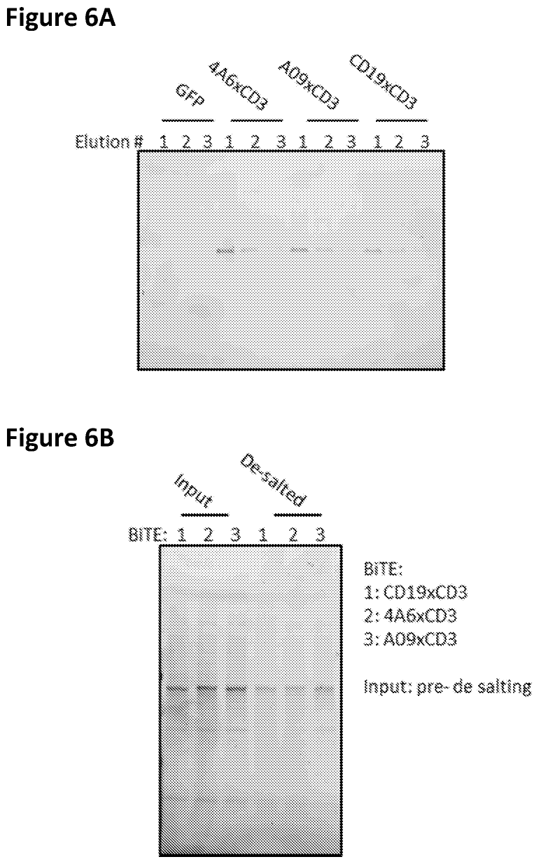

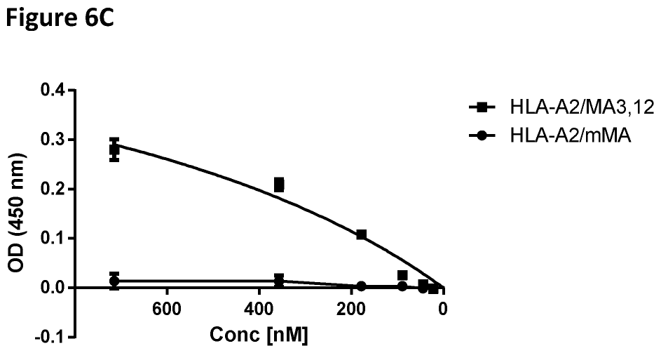

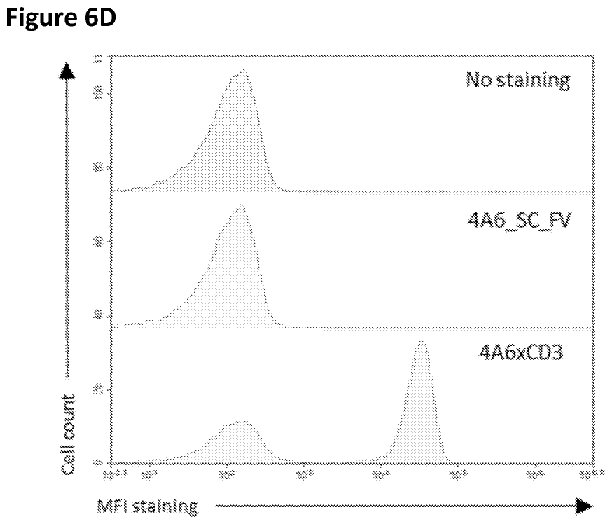

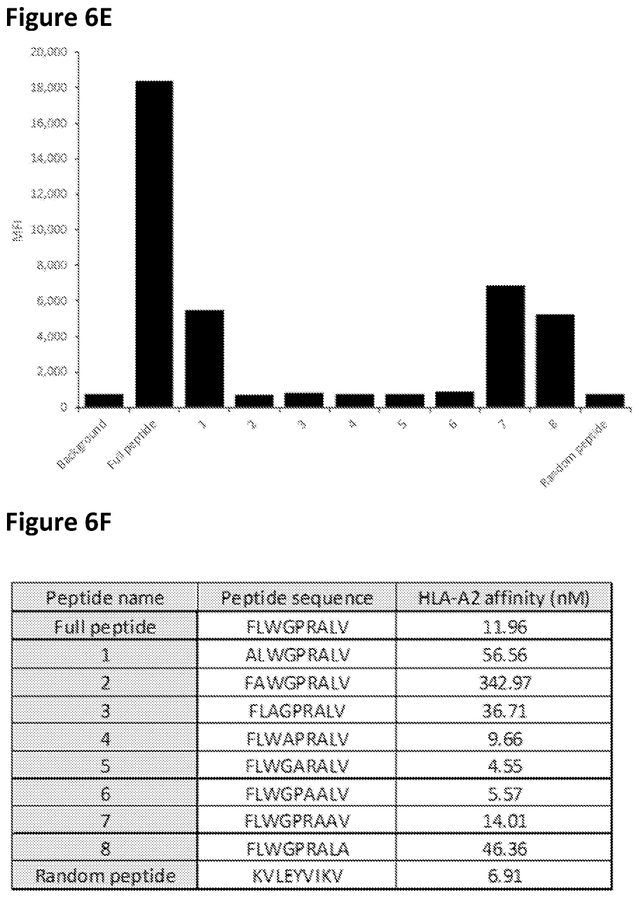

Bispecific molecules were produced in 293F cells transfected with the appropriate pFuse expression vectors at a cell density between 1 and 2 million cells per ml. Transfected cells were allowed to recover for 2 days at 37° C., followed by an incubation at 30° C. for four days during which the bispecific molecules were secreted in the medium. Bispecific molecules were purified from the medium using either Ni-NTA (Thermo Scientific) or Talon beads (Clontech) according to manufacturer's instructions. Upon purification of the molecules, clear bands corresponding to bispecific molecules were visualized on SDS-PAGE as shown in FIGS. 6A and B. ELISA assay was used to confirm the specificity of expressed molecules towards HLA-A2/MAGE-A derived peptide complexes. Biotinylated HLA-A2/MA_{3, 12} (FLWGPRALV) and HLA-A2/mMA (YLEYRQVPG) complexes were coated in a 96 well plate. 4A6×CD3 (SEQ ID NO: 63) at decreasing concentration was incubated and allowed to bind the complexes, followed by an incubation with anti-his-HRP antibody (Thermo Scientific). The binding of bispecific molecule was visualized by incubation with 3, 3′,5,5′-Tetramethylbenzidine (Thermo Scientific) followed by absorbance measurement at OD450. The results shown in FIG. 6C confirm the specificity of 4A6×CD3 towards HLA-A2/MA_{3, 12}.

8.2 Binding of the Bispecific Molecule of the Disclosure to Immune Cells

Binding of 4A6×CD3 (SEQ ID NO: 63) to CD3 molecule expressed by T cells was established in a flow cytometric assay by incubating 200.000 peripheral blood mononuclear cells (PBMCs) with 50 ng/ml 4A6×CD3 or 4A6_SC_FV (monospecific antibody fragment used here as a negative control). Flow cytometric analysis showed only binding of 4A6×CD3 to the PBMCs and not of control molecule 4A6_SC_FV (FIG. 6D). 4A6_SC_FV molecule binds specifically to HLA-A2/MA_{3, 12} and does not bind CD3 molecule present on immune cells (as shown by lack of signal shift in the middle histogram of FIG. 6D). This result confirmed that 4A6×CD3 specifically recognizes CD3 molecule expressed on the surface of T cells.

8.3 Determination of 4A6×CD3 Fine Specificity

Fine specificity of the bispecific molecule was assessed by pulsing 200.000 JY cells overnight under serum free conditions with 100 μg/ml peptide variants. The amino acids of the used peptides were sequentially substituted for an alanine. Pulsed JY cells were incubated with constant concentration of 4A6×CD3 (SEQ ID NO: 63). The binding of the 4A6×CD3 was detected upon incubation with anti-his antibody. The obtained binding pattern presented in FIG. 6E showed that all peptide amino acids contribute to the binding of 4A6×CD3 to the HLA-A2/MA_{3, 12} complex and that amino acids at positions 2 till 6 were of particular importance for the binding of 4A6×CD3 towards the HLA-A2/MA_{3, 12} complex. Table presented in FIG. 6F lists amino acid sequences of all peptides used in this alanine scan experiment, as well as their predicted affinity to HLA-A2.

### Example 9

T-Cell Activation by the Bispecific Molecule of the Disclosure

9.1 Bispecific Molecules of Disclosure Lead to T Cell Activation in Presence of H1299 Cells Stably Expressing MAGE-A Derived Peptides in Complex with HLA

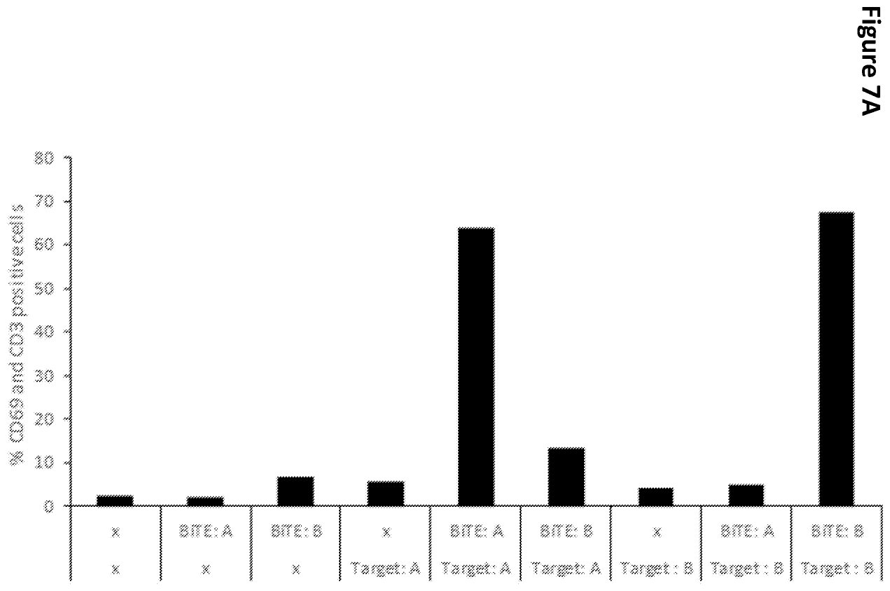

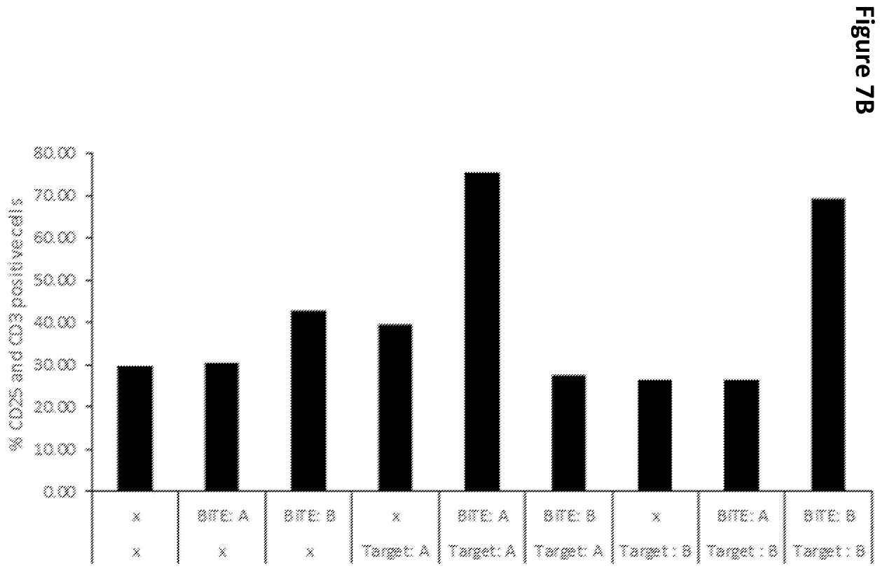

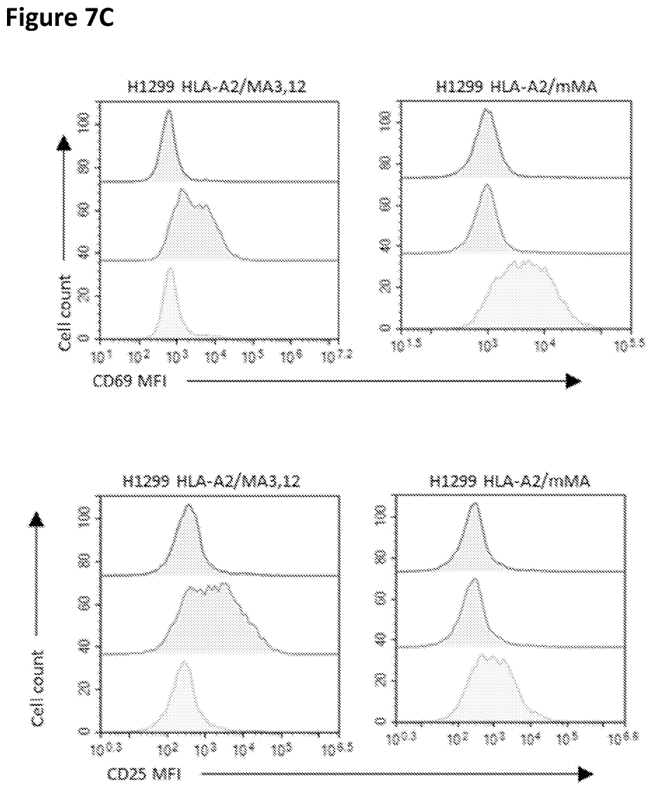

Non small cell lung carcinoma H1299 cells transfected to stably express respective MAGE-A derived peptides in complex with HLA, further referred to as target cells, were seeded and allowed to attach to the culture plate overnight. Next day the cell culture medium was refreshed and PBMCs (effector cells) and bispecific molecules of disclosure at concentration of 500 ng/ml were added. The assay was performed at target to effector cells ratio of 1:16 with a 72 hour incubation. Both target and effector cells were harvested. A flow cytometric analysis was performed in order to detect expression of T-cell activation markers (CD69 and CD25). Results plotted as % of CD3 positive cells expressing CD69 or CD25 are shown in FIGS. 7A and 7B, respectively. Specific increase in both T-cell activation markers was observed only when PBMCs were incubated with bispecific molecules and respective target cells. Incubation of PBMCs and target cells in absence of bispecific molecules did not lead to increase of CD69 or CD25 expression. Histograms presented in FIG. 7C confirm that specific T cell activation takes place only in presence of bispecific molecules, target cells and PBMCs.

9.2 T Cell Activation is Dependent on Bispecific Molecule Concentration

Respective target cells were seeded and allowed to attach to the culture plate overnight. Next day the cell culture medium was refreshed and PBMCs (effector cells) as well as bispecific molecules of disclosure at increasing concentration were added. The assay was performed at target to effector cells ratio of 1:16 with a 72 hour incubation. Both target and effector cells were harvested. A flow cytometric analysis was performed in order to detect expression of T-cell activation markers (CD69 and CD25). Specific increase in both T-cell activation markers was observed when PBMCs were incubated with either 4A6×CD3 (SEQ ID NO: 63) or A09×CD3 (SEQ ID NO: 64) with respective target expressing cell line (FIG. 7D). Increase of T-cell activation was observed with increase of bispecific molecule's concentration.

9.3 Effect of Target to Effector Cells Ratio on T Cell Activation

When target cells were incubated with a constant concentration of bispecific molecule (500 ng/ml) and varying target to effector ratios for 72 hours (FIG. 7E), no difference in level of T-cell activation determined as expression level of CD69 and CD25 was observed. 9.4 Formation of immune synapse Formation of immune synapses was observed upon microscopic inspection of cells used in assays described under 9.1-9.4. The physical attraction of immune cells to target cells shown in FIG. 7F was observed after 24 hours only in the samples which showed increase of T-cell activation markers measured by flow cytometry.

9.5 Formation of Immune Synapse is Dependent on Presence of BiTE Specifically Recognizing HLA/MAGE Complexes on Surface of Targeted Cells

H1299 cell stably expressing HLA-A2/FLWGPRALV and H1299 cells stably expressing HLA-A2/YLEYRQVPG were seeded at density of 50,000 cells per well and allowed to adhere for 16 hours. Next, medium was refreshed (FIG. 7G, mock) and optionally medium containing PBMCs only (FIG. 7G, PBMC), or PBMC and BiTE (FIG. 7G, PBMC+A09×CD3 and PBMC+A09×CD3) were added. The assay was performed for 72 hours at 37° C., at target to effector cells ratio of 1:16 (in conditions were H1299 transfectants were co-cultured with PBMC). Both 4A6×CD3 (SEQ ID NO: 63) or A09×CD3 (SEQ ID NO: 64) were added to a final concentration of 500 ng/ml. At the end of the assay phase contrast images were taken in order to assess HLA/MAGE target specific formation of immune synapse facilitated by respective BiTE molecule. Immune synapse formation was observed in wells in which H1299 cells stably expressing HLA-A2/FLWGPRALV were co-cultured with PBMC in presence of 4A6×CD3 and in wells in which H1299 cells stably expressing HLA-A2/YLEYRQVPG were co-cultured with PBMC in presence of A09×CD3. Arrows in FIG. 7G point to examples of immune synapse formation. No immune synapse formation was observed when PBMC were co-cultured with transfected H1299 cells in absence of bispecific molecules of the disclosure. Phase contrast images show that immune synapse formation is dependent on HLA/MAGE recognition by BiTE.

### Example 10. Bispecific Molecules of Disclosure Lead to T Cell Activation

10.1. Bispecific Molecules of Disclosure Lead to T Cell Activation in Presence of 911 Cells Stably Expressing MAGE-A Derived Peptides in Complex with HLA

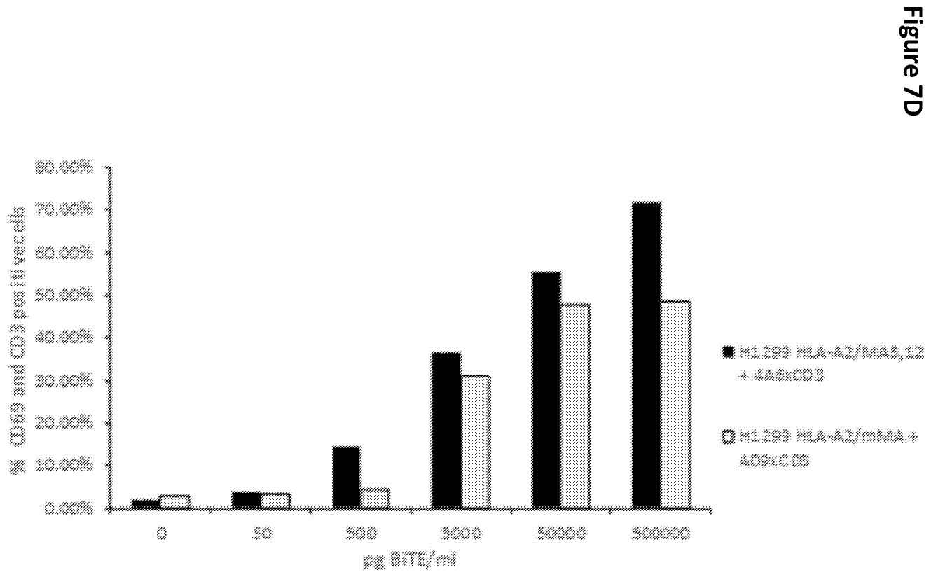

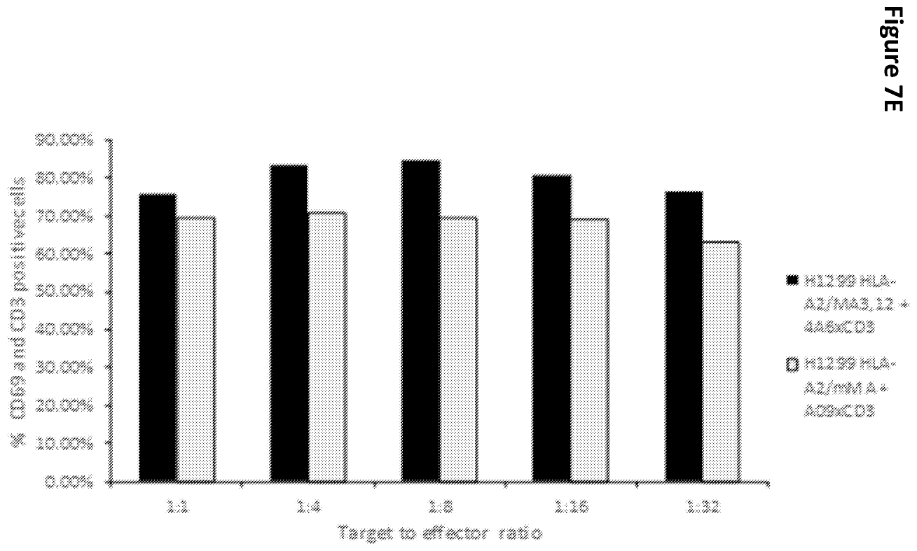

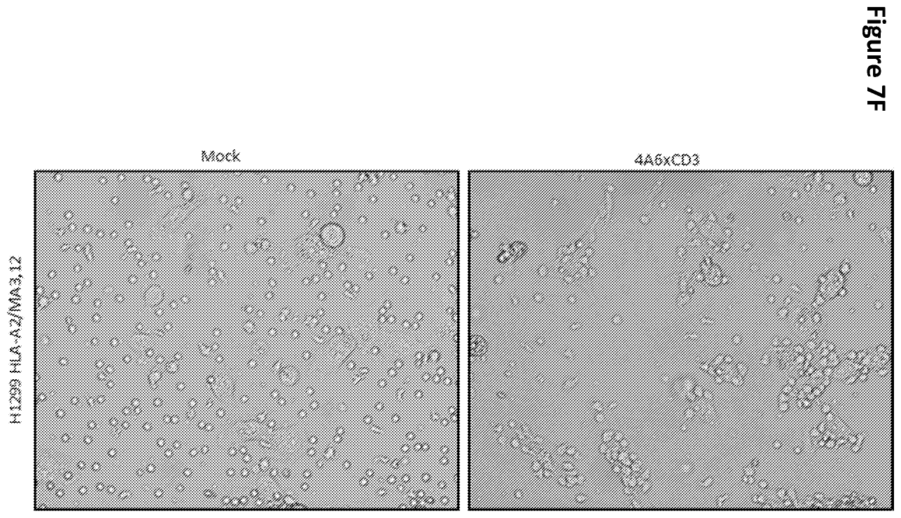

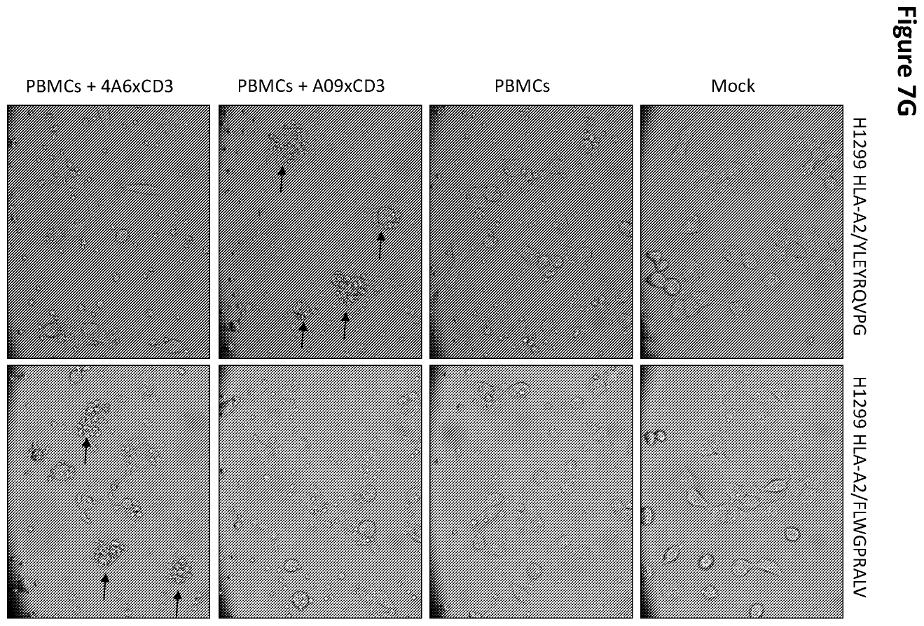

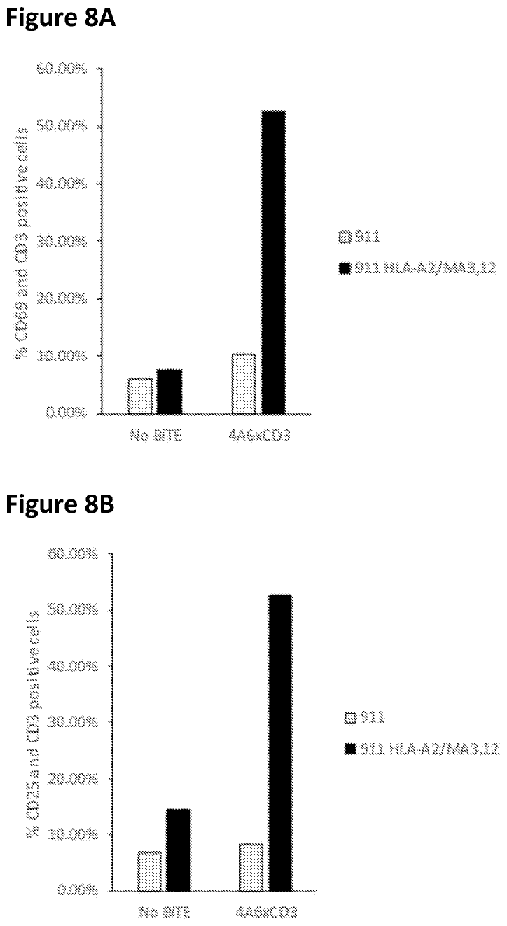

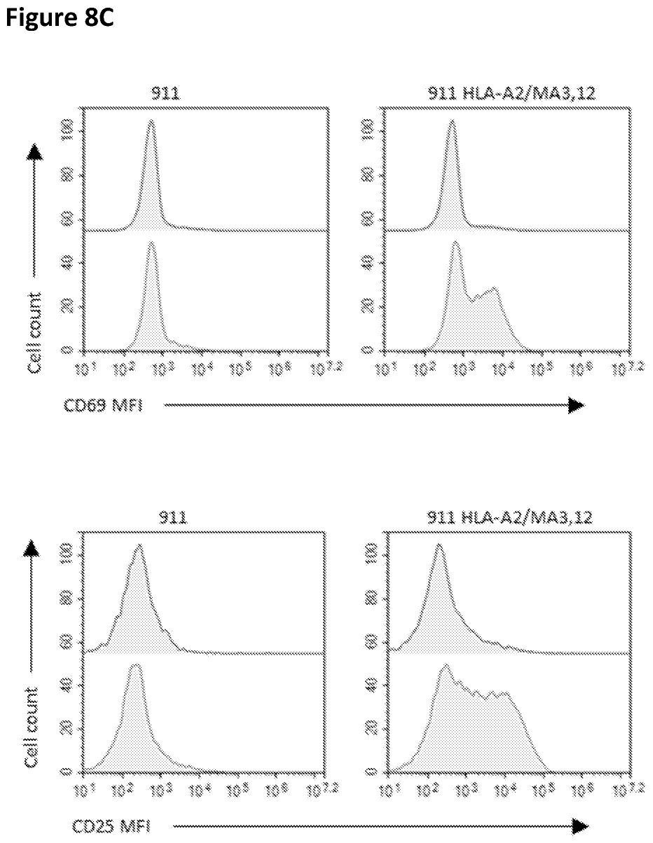

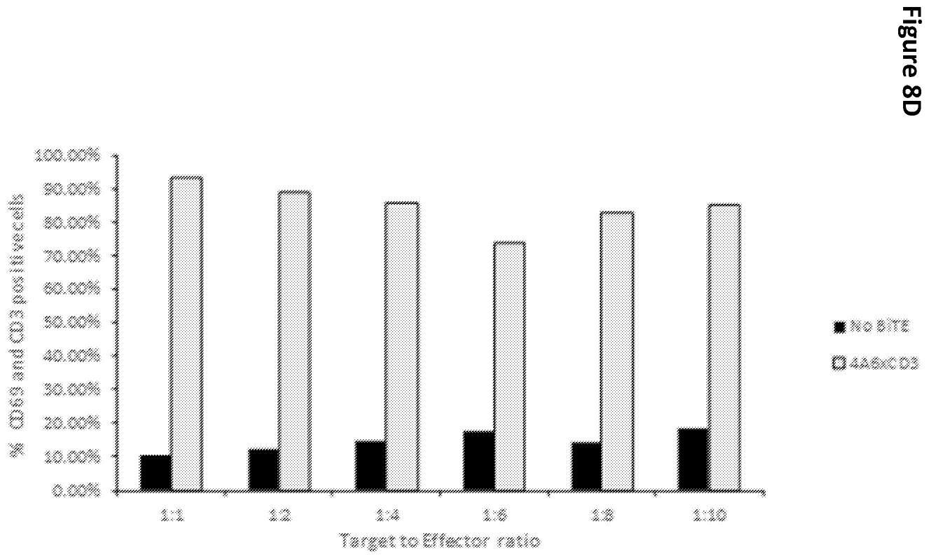

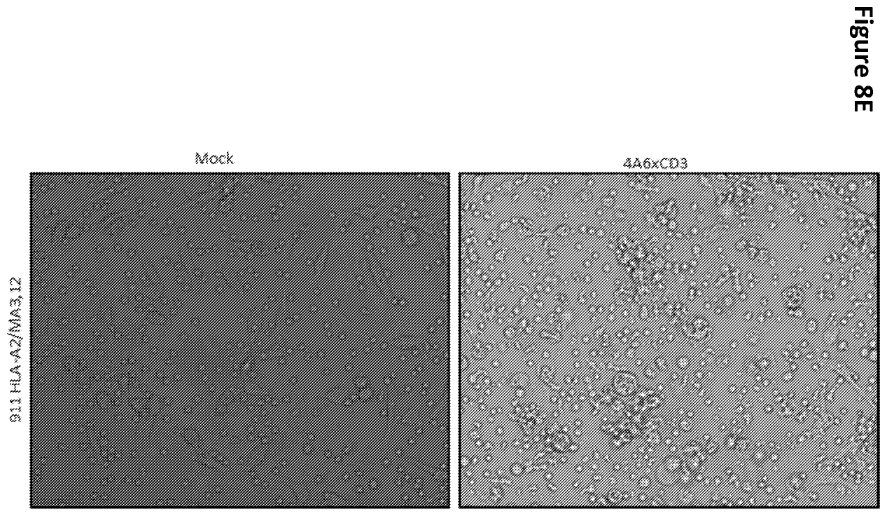

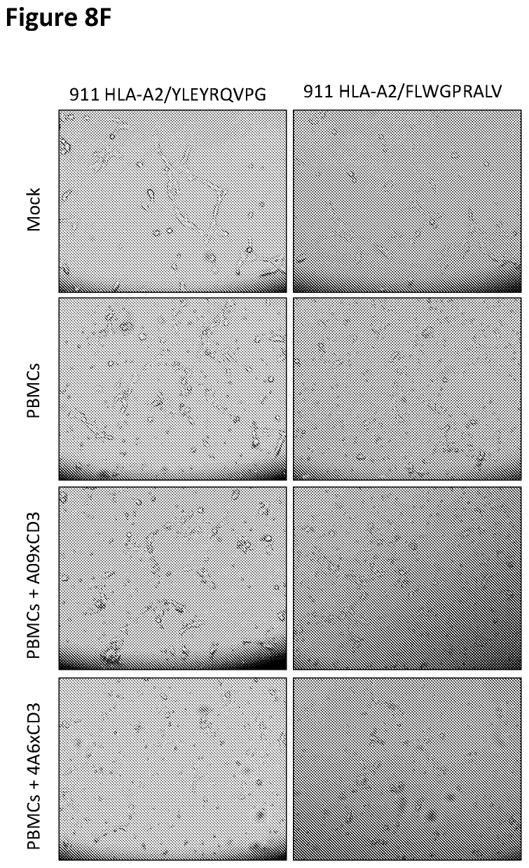

Transformed human embryonic retina cells (911) transfected to stably express respective MAGE-A derived peptides in complex with HLA, further referred to as target cells, were seeded and allowed to attach to the culture plate overnight. Next day the cell culture medium was refreshed. PBMCs (at a target to effector ratio of 1:8) and 4A6×CD3, SEQ ID NO: 63, (at 500 ng/ml) were added and incubated for 72 hours. Both target and effector cells were harvested. Flow cytometric analysis of effector cells showed increase in expression of T-cell activation markers CD69 and CD25. Results plotted as % of CD3 positive cells expressing CD69 or CD25 are shown in FIGS. 8A and 8B, respectively. Histograms presented in FIG. 8C confirm that specific T cell activation takes place only in presence of bispecific molecules, target cells and PBMCs, as shown by the clear shift in the MFI signal recorded under those conditions.

10.2 Effect of Target to Effector Cells Ratio on T Cell Activation

When target cells were incubated with a constant concentration of bispecific molecule (500 ng/ml) and varying target to effector ratios for 72 hours, no difference in level of T-cell activation determined as expression level of CD69 and CD25 was observed (FIG. 8D).

During the assays described above target cells could hardly be observed after 72 hours in the conditions showing T-cell activation.

10.3 Formation of Immune Synapse

Formation of immune synapse was observed in assays described under 10.1 and 7102. The physical attraction of immune cells to target cells as shown in FIG. 8E was observed after 24 hours only in the samples which showed increase of T-cell activation markers measured by flow cytometry. Upon 72 hour incubation of target cells with effector cells in presence of bispecific molecule, hardly any target cells remained. FIG. 8F depicts interaction of PBMC with HLA/MAGE target complex expressing 911 cells in presence or absence of bispecific molecules. Here, the target complex expressing 911 cells and 911 cells expressing control HLA-A2/MAGE complex were seeded at the density of 50,000 cells per well, allowed to adhere for 16 hours and incubated with bispecific molecules (500 ng/ml) at target to effector ratio of 1:16 for 24 hours. A09×CD3 BiTE binds 911 cells stably expressing HLA-A2/YLEYRQVPG complex (i.e. HLA-A2/mMA), whereas 4A6×CD3 BiTE binds 911 cells stably expressing HLA-A2/FLWGPRALV complex (i.e. HLA-A2/MA_{3, 12}). In this assay 911 cells stably expressing HLA-A2/FLWGPRALV complex serve as specificity control for A09×CD3 BiTE, whereas 911 cells stably expressing HLA-A2/FLWGPRALV complex serve as specificity control for 4A6×CD3 BiTE. In FIG. 8F it is shown that in conditions devoid of BiTE the interactions between 911 transfectants and PBMCs are rare. Similarly, such interactions are rare in conditions in which BiTE molecules were incubated with PBMCs in presence of 911 cells not expressing the target HLA/MAGE complex in their surface. Only when PMBCs were incubated with either 4A6×CD3 (SEQ ID NO: 63) or A09×CD3 (SEQ ID NO: 64) and 911 transfectants expressing the respective, targeted by BiTE molecules, HLA/MAGE complexes, an increased number of immune synapses formed by PBMCs and 911 cells was observed. These BiTE-induced interactions often resulted in change of transfected 911 cells' morphology.

### Example 11

T-Cell Activation Upon Incubation with A09×CD3 and Glioblastoma Cells.

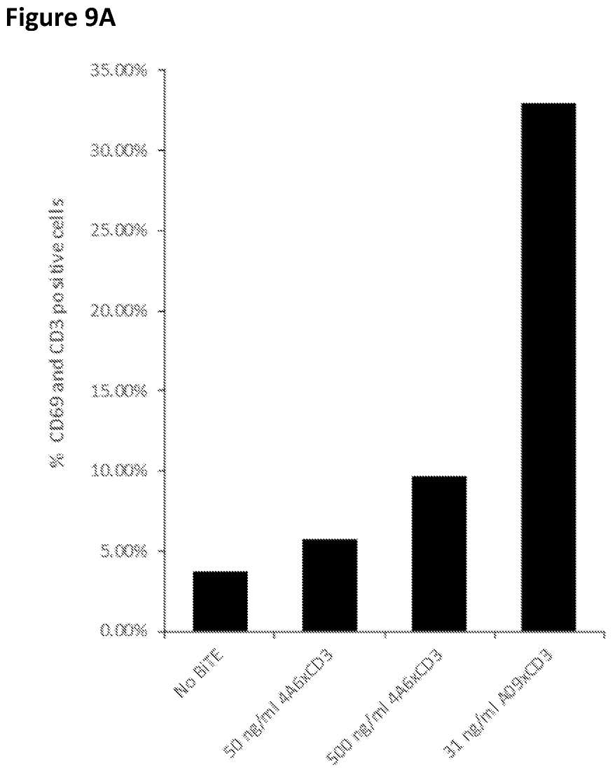

U87 cells, which express both MAGE-A and HLA-A2 proteins, were seeded and allowed to attach to culture plate overnight. Next day the culture medium was refreshed. PBMCs were added at target to effector ratio of 1:8, whereas bispecific molecule 4A6×CD3 (SEQ ID NO: 63) was added at a final concentration of either 50 ng/ml or 500 ng/ml and A09×CD3 (SEQ ID NO: 64) at 31 ng/ml. The incubation lasted for 72 hours. Both target and effector cells were harvested and analysed by flow cytometry. Expression of T-cell activation marker CD69 was evaluated. Specific increase in expression of T cell activation markers plotted in FIG. 9A was observed when PBMCs were incubated with A09×CD3 in presence of U87 cells. A clear shift in the MFI signal recorded under those conditions is presented in histograms of FIG. 9B.

### Example 12

4A6×CD3 and A09×CD3 Induce Apoptosis of H1299 Transfectants but not of HLA-A2 Positive Human Pulmonary Fibroblasts.

Non small cell lung carcinoma H1299 cells transfected to stably express respective MAGE-A derived peptides in complex with HLA (HLA-A2/FLWGPRALV or HLA-A2/YLEYRQVPG) as well as human pulmonary fibroblasts (HPFs) were seeded at the density of 100,000 cells per well. After 16 hours incubation at 37° C., 800′000 peripheral blood mononuclear cells (PBMCs) were added in presence or absence of respectively 500 ng/ml 4A6×CD3 (SEQ ID NO: 63) or A09×CD3 (SEQ ID NO: 64). The cells were further incubated for up to 72 hours at 37° C. The cells were harvested using trypsinization and lysed on ice using lysis buffer (150 mM NaCl, 25 mM Tris-HCl, 10 mM EDTA, 1% Triton X-100, 1× Protease inhibitor cocktail). BCA assay was used to assess total protein concentration. Western blot was performed in order to detect cleaved poly-ADP-ribose polymerases (PARP) and actin. PARP is one of several known cellular substrates of caspases. Its cleavage by caspases is considered to be a hallmark of apoptosis. Following antibodies were used: rabbit-anti-cleaved PARP (Cell Signaling Technologies, cat. no. 5625S), goat-anti-rabbit-HRP (SC-2004, Santa Cruz), mouse anti-beta Actin (AB8226, Abcam), goat-anti-mouse-HRP (SC-516102-CM, Santa Cruz). Antibody binding was imaged by Chemidoc Imaging System (BioRad). Bands corresponding to cleaved PARP were only visualized in lysate samples derived from H1299 cells expressing HLA-A2/FLWGRPALV co-cultured with PBMCs and 4A6×CD3, and in lysate samples of H1299 cells expressing HLA-A2/YLEYRQVPG co-cultured with PBMCs and incubated with A09×CD3 (FIG. 10A). No bands corresponding to cleaved PARP were detected in lysate samples of HPFs co-cultured with PMBC in presence of 4A6×CD3 or A09×CD3 (FIG. 10B). This result shows HLA/MAGE specific induction of apoptosis as only when cells expressing HLA/MAGE complex of interest were incubate with PBMC and respective BiTE molecule (either 4A6×CD3 or A09×CD3) apoptotic marker, cleaved PARP, was detected.

### Example 13

13.1 A09×CD3 and 4A6×CD3 Facilitate Attraction of Immune Effector Cells to Target Cells Resulting in Decreased Numbers of the Latter and Induction of Apoptosis

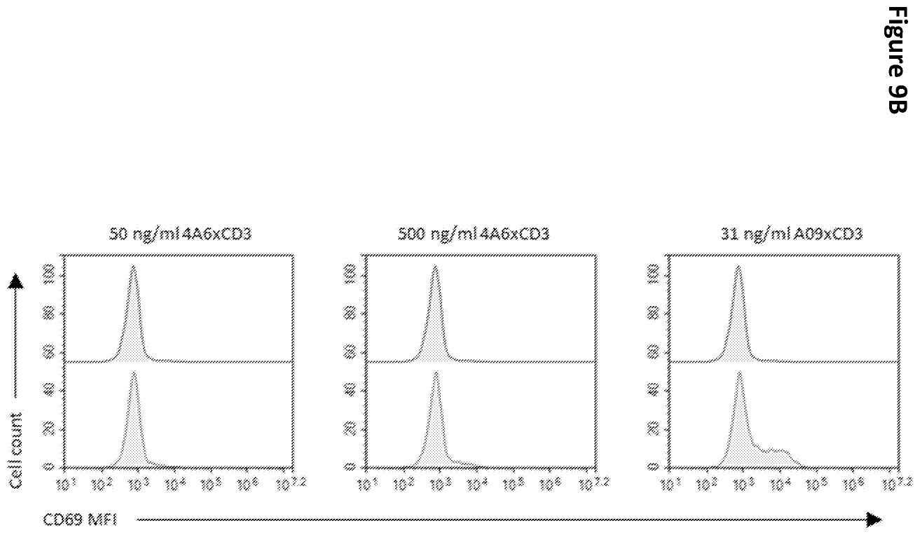

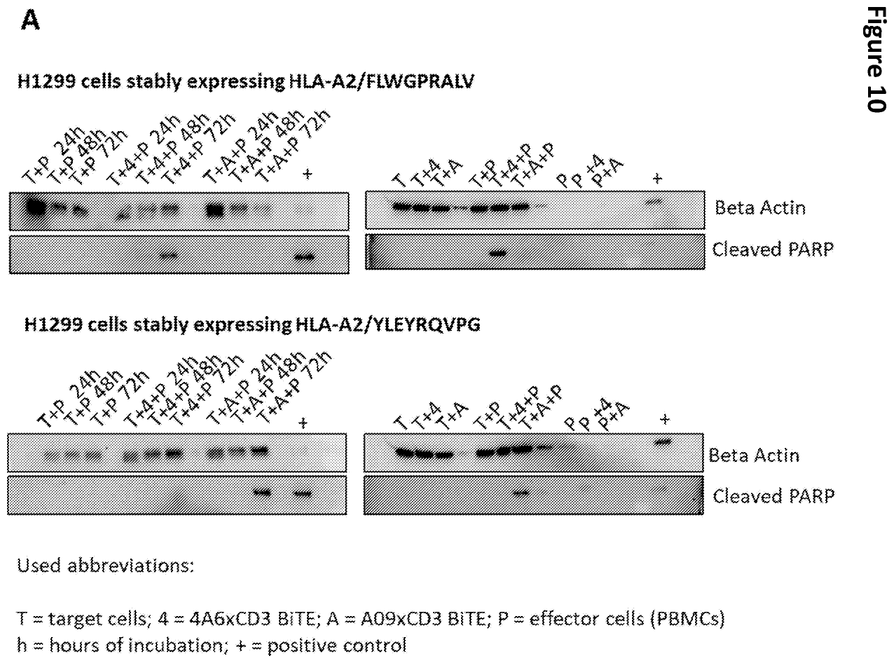

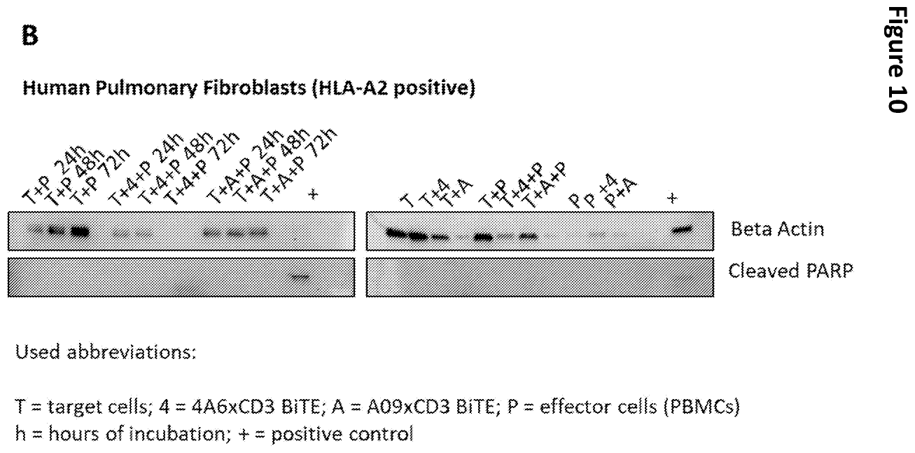

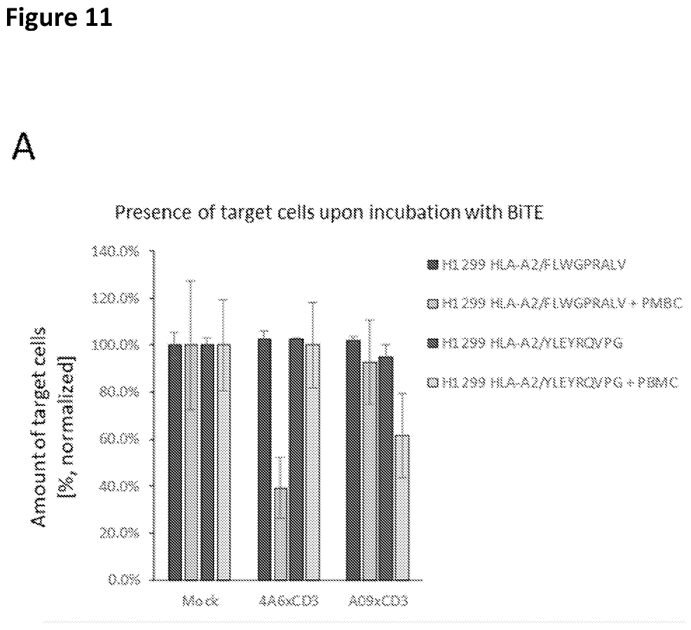

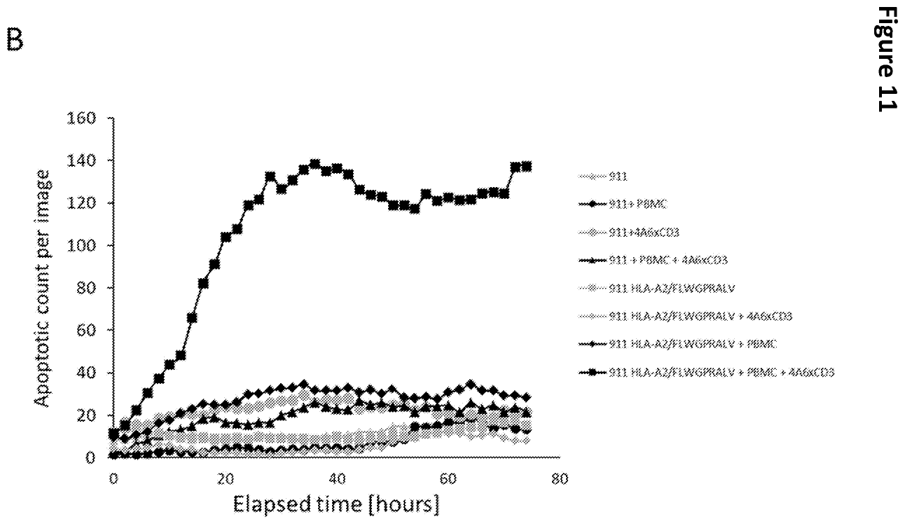

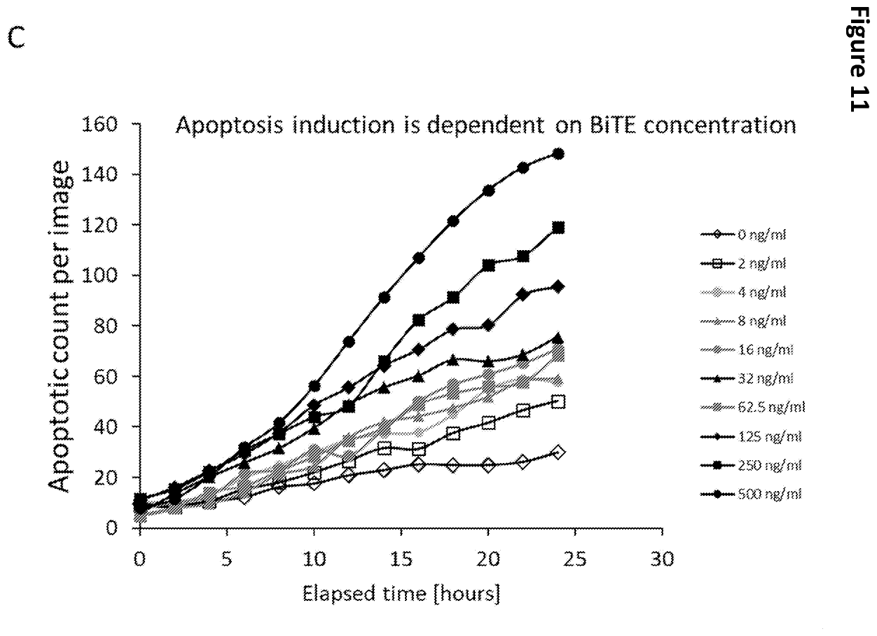

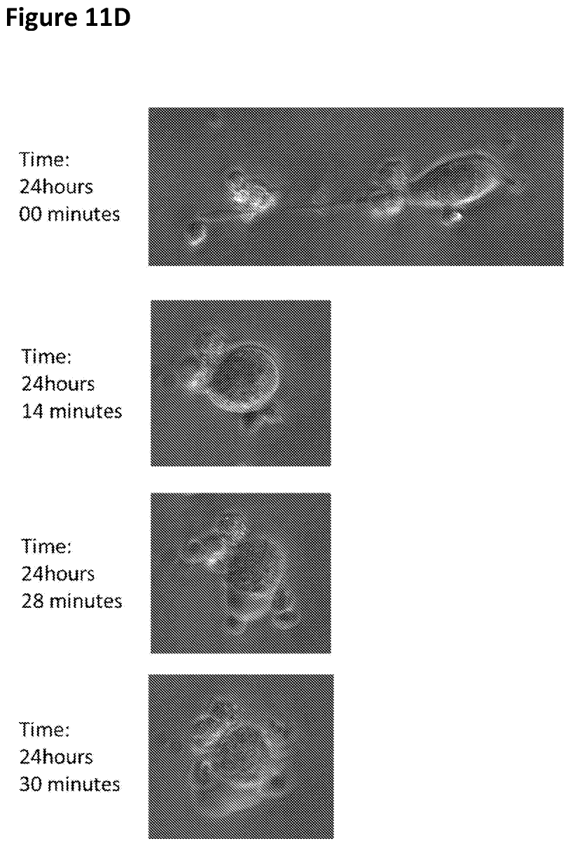

Reduced numbers of H1299 target overexpressing cells, namely H1299 stably expressing HLA-A*0201/FLWGRPALV and H1299 stably expressing HLA-A*0201/YLEYRQVPG, were observed during flow cytometric analysis. 4A6×CD3 (SEQ ID NO: 63) binds CD3 on surface of immune effector cells and HLA-A*0201/FLWGRPALV on surface of transfected H1299, whereas A09×CD3 (SEQ ID NO: 64) binds CD3 on surface of immune effector cells and HLA-A*0201/YLEYRQVPG on surface of transfected H1299 cells. H1299 cells stably expressing HLA/MAGE-derived peptide complexes of interest were incubated for 3 days with PMBC (target to effector ratio 1:16) in presence or absence of respective BiTE molecules of the disclosure (at 500 ng/ml). Target cells were identified during the flow cytometric analysis based on their size. Decrease in number of target cells was observed only in conditions in which PBMC were co-cultured either with H1299 stably expressing HLA-A*0201/FLWGRPALV in presence of 4A6×CD3, or with H1299 stably expressing HLA-A*0201/YLEYRQVPG in presence of A09×CD3. No reduction in number of H1299 stably expressing HLA/MAGE-derived peptide complexes was observed in control conditions (FIG. 11A).

13.2 Incucyte Live Cell Imaging Shows that Apoptosis Induction by Immune Effector Cells is Dependent on Concentration of BiTE Molecules' of Disclosure.

Incucyte live cell imaging was used to measure in time number of apoptotic events. The 911 cells stably expressing HLA-A*0201/FLWGPRALV complexes or non transfected 911 cells were seeded one day prior to start of imaging. Next day, medium was refreshed and PBMCs were added in presence or absence of 4A6×CD3 BiTE (SEQ ID NO: 63). Various target to effector cell ratios were tested, as well as various concentrations of 4A6×CD3 BiTE (from 2 ng/ml to 500 ng/ml). Apoptotic events were visualized by Cell Event™ Caspase-3/7 Green Detection Reagent (Essen BioScience, cat. no. 9500-4440-E00). Co-cultures of target and effector cells were imaged every 2 hours for 72 hours and activation of caspase 3/7 was used to detect apoptotic events. The analysis showed a rapid onset of apoptosis of 911 target HLA/MAGE-derived peptide complex expressing cells. The detection of apoptosis only occurred when 4A6×CD3 was incubated with a co-culture of 911_cells stably expressing HLA-A*0201/FLWGPRALV cells and PBMCs. This confirms the specificity of 4A6×CD3 BiTE as no caspase activation was detected when 911 cells, devoid of target HLA/MAGE-derived peptide complex, were incubated with PBMC in presence of 4A6×CD3 BiTE (FIG. 11B). Activation of caspases was observed almost immediately after the onset of the experiment and continuously increased in the conditions which combined PBMCs, 4A6×CD3 and 911 cells stably expressing HLA-A*0201/FLWGPRALV, but not in conditions with a 911 cell line devoid of HLA-A*0201/FLWGPRALV. A 4A6×CD3 BiTE concentration dependency of increase rate and maximum induced caspase 3/7 activation was observed (FIG. 11C). All tested 4A6×CD3 BiTE concentrations facilitated induction of apoptosis by immune effector cells, even as low as 2 ng/ml. Although all 4A6×CD3 BiTE concentrations induced apoptosis, concentrations of 500 ng/ml and 250 ng/ml of 4A6×CD3 BiTE resulted in a sharp increase of apoptotic signal which saturated after approximately 30 hours after start of incubation.

13.3 High Resolution Live Cell Imaging

High resolution live cell imaging was performed and showed the induction of apoptosis in 911 cells stably expressing HLA-A*0201/FLWGPRALV complexes. 911 cells stably expressing HLA-A*0201/FLWGPRALV were seeded per well (at density of 1,500 per well) and allowed to attach for 16 hours. Next day, the cell culture medium was refreshed and 12,000 PBMC were added per well. Cells were co-cultured in presence of 500 ng/ml of 4A6×CD3 BiTE (SEQ ID NO: 63) and Cell Event™ Caspase-3/7 Green Detection Reagent (Essen BioScience, cat. no. 9500-4440-E00). After 24 hour incubation the live cell imaging measurement was started. Phase contrast and fluorescent images (in the GFP channel) were acquired every 2 minutes for 4 hours. FIG. 11D shows a time course, in which effector cells are attracted to 911 cells stably expressing HLA-A*0201/FLWGPRALV (here used as target positive cells), the morphological changes of the target positive cells are shown in consecutive images (the target cells round up and after 30 minutes from start of imaging show membrane blebbing which is a sign of apoptosis). The fluorescent signal increases in time as caspase activation occurs.

### Example 14

Immune Effector Cells Secrete Cytokines when Co-Cultured with Cells Expressing HLA/MAGE-A Derived Peptide Complexes in Presence of Bispecific Molecules of the Disclosure

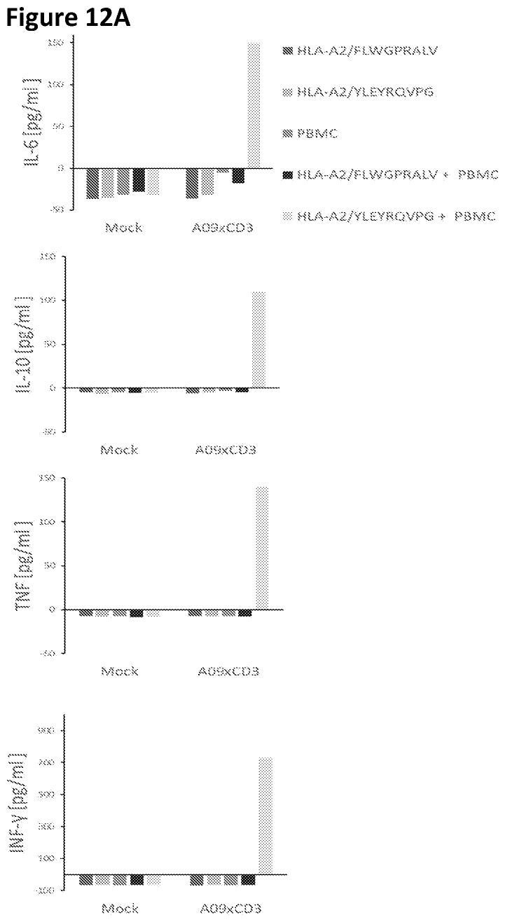

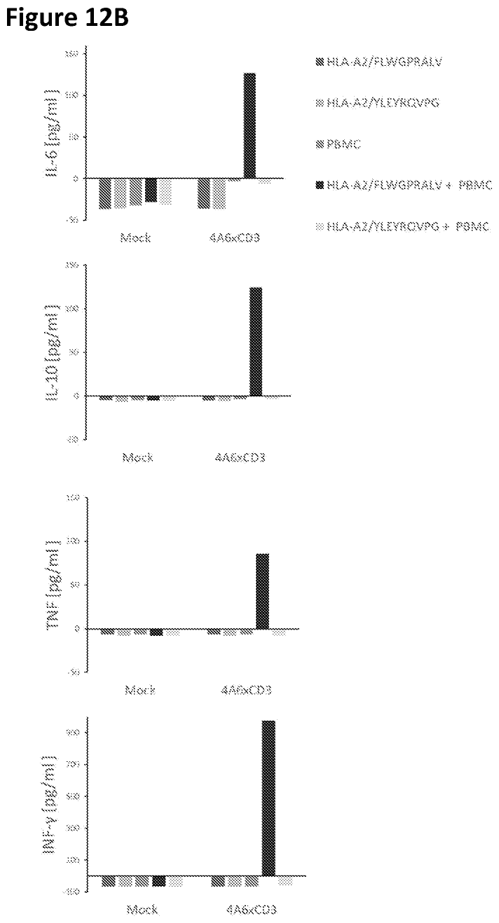

Upon demonstrating the expression of specific T cell activation markers (CD69 and CD25) in presence of respective BiTEs and cells expressing HLA/MAGE-A complexes, the cytokines released by T cells were measured using BD Cytometric Bead Array (CBA) Human Th1/Th2/Th17 Cytokine Kit (BD, catalog no. 560484). Briefly, transfected H1299 cells stably expressing respective HLA-A*0201/MAGE-derived peptide complexes were seeded at the density of 50,000 cells/well and allowed to attach overnight. Next day, cells were washed and medium was refreshed. PBMC were added and cells were co-cultured in presence or absence of BiTE molecules of the disclosure (500 ng/ml) for 72 hours at effector to target ratio of 16:1. At the end of the incubation period, the cell culture supernatants were collected and analyzed by flow cytometry for presence of following cytokines Interleukin-2 (IL-2), Interleukin-4 (IL-4), Interleukin-6 (IL-6), Interleukin-10 (IL-10), Tumor Necrosis Factor (TNF), Interferon-γ (IFN-γ), and Interleukin-17A (IL-17A) according to instruction provided in the BD Cytometric Bead Array (CBA) Human Th1/Th2/Th17 Cytokine Kit (BD, catalog no. 560484). As shown in FIG. 12 A09×CD3 (A) and 4A6×CD3 (B) BiTE molecules were capable of inducing INFγ secretion (˜1000 μg/ml) by PBMCs when incubated in co-cultures with the respective target expressing cell line. A similar specific cytokine secretion was observed for IL-6, IL-10 and TNF, yet at approximately 10 fold lower concentrations. Co-incubation of target expressing cells with PBMC in presence of BiTE did not result in induction of IL-17A, IL-2 or IL-4 secretion. These results further support the presence of a specific immune cell activation.

### Example 15

4A6×CD3 BiTE Directs Human PBMC to Xenograft of H1299 Cells Stably Expressing HLA-A*0201/FLWGPRALV In Vivo.

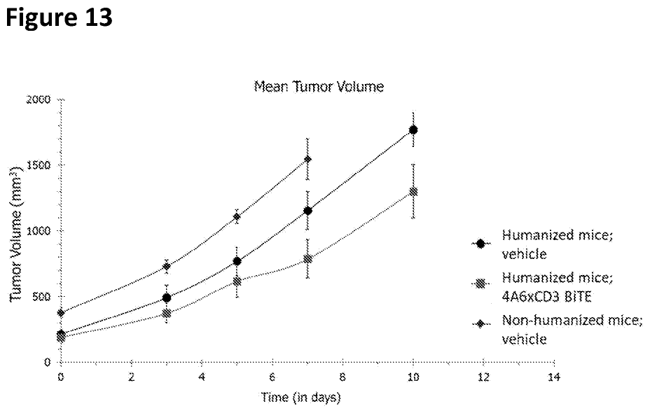

Female NSG-B2m mice (NOD.Cg-B2^{mtm1Unc} Prkdc^{scid}//2rg^{tm1wjl/}SzJ), 6- to 9-week-old were provided by Charles River Laboratories. Mice were housed in boxes to a maximum of 6 animals during acclimation period and to a maximum of 6 animals during the experimental phase. Each mouse was offered a complete pellet diet and filtered, sterilized tap water ad libitum throughout the study. On the day of tumor graft induction, 200×10⁶ of H1299 cells stably expressing HLA-A*0201/FLWGPRALV derived cells were centrifugated at 150 g for 5 minutes and resuspended in 4 ml of cell culture medium (RPMI-1640 medium without FBS) to a final concentration of 50×10⁶ cells/ml. Just before injection, cell suspension was mixed with 4 ml Matrigel. Mice were anaesthetized with 100 mg/kg ketamine hydrochloride and 10 mg/kg xylazine, and then skin was aseptized with a chlorhexidine. 100 μl of cell suspension, containing 2.5×10⁶ cells, were injected in the interscapular fat pad. Mice are allocated to the different groups according to their tumor volume, to obtain groups with homogenous mean and median tumor volumes. After 7 days mice with palpable tumors were injected intraperitoneally with human PBMCs (20×10⁶). 10 days post PBMC injection, when tumors' volume [mm³] range of 62.5-288 was reached, animals were enrolled into study groups [n=7]. Group 1 was injected daily for 14 days with vehicle and group 2 was injected daily with 19.8 μg of 4A6×CD3 BiTE (SEQ ID NO: 63) for 14 days. Animals weight and tumor volume were measured 3 times a week. Animals were sacrificed when tumor volume was equal to or exceeded 1764 mm³. From all mice blood samples were collected regularly after humanization (i.e. after PBMC injection) as well as blood samples were collected at ethical sacrifice for cytometry analysis of human immune cells. From 4 mice from each group tumors were collected at ethical sacrifice for cytometry analysis of human immune infiltrated cells. Animals injected with 4A6×CD3 BiTE showed a slowed tumor growth in comparison to tumor growth in control animals (FIG. 13).

### Example 16

### Production of Bi-Specific Nanobodies

Nanobody phage display selections were performed using decreasing concentrations of biotinylated HLA-A*0201/YLEYRQVPG as antigen and MCF7 phage display libraries (preparation of the libraries was described in Kijanka M, Warnders F J, El Khattabi M, Lub-de Hooge M et al. Eur J Nucl Med Mol Imaging. 2013 October; 40(11):1718-29). Phages were eluted with 0.1M TEA and showed binding to biotinylated complexes of HLA-A*0201/YLEYRQVPG in phage ELISA. Phagemid vectors were sequenced to retrieve nanobody encoding nucleotide sequences. Table 1 provides overview of VHH sequences binding MAGE derived peptides in complex with HLA-A*0201.

TABLE 1

| SEQ ID NO | DESCRIPTION |
|---|---|
| 47 | 2G5 |
| 48 | 3B6 |
| 49 | 4F5 |
| 50 | 4D2 |
| 51 | 3D10 |
| 52 | 1B10 |
| 53 | 1F5 |
| 54 | 3A3 |
| 55 | 2C5 |
| 56 | 1F11 |

Sequence encoding nanobody, referred to as 1B10 (SEQ ID NO: 52) was used to generate a nanobody bispecific construct in which the N-terminal nanobody bound HLA-A*0201/YLEYRQVPG complex presented on the surface of tumor cells and was connected via a G4S linker to C-terminal nanobody which bound CD3 expressed on the surface of immune effector cells. The amino acid sequence of the VHH binding CD3 expressed on the surface of immune effector cells is listed as SEQ ID NO: 57, whereas the amino acid sequence of the 1B10×CD3 bispecific nanobody is listed as SEQ ID NO: 58.

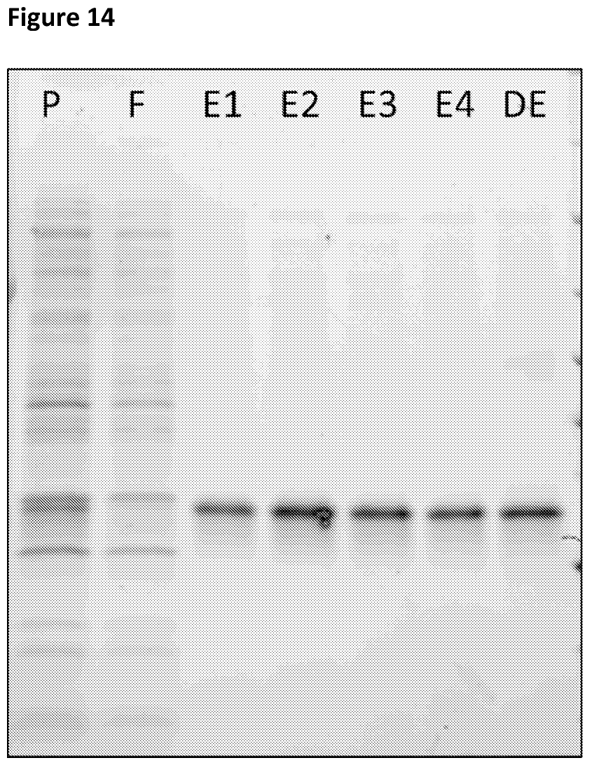

BL21 cells were grown in 2YT medium at 37° C. until a logarithmic growth phase was reached. Isopropyl β-D-1 thiogalactopyranoside (IPTG) was added to the medium to a final concentration of 1 mM to induce production of bispecific nanobody molecule. Upon addition of IPTG temperature was decreased to 25° C. and incubation continued for 16 hours. At the end of incubation cells were pelleted by centrifugation (15 minutes at 400 g) and resuspended in PBS. To isolate produced nanobodies bacterial cell pellet was subjected to three freeze thaw cycles. Cellular debris was removed by centrifugation (15 minutes at 4000 g). Supernatant containing produced nanobody was subjected to incubation with NiNTA beads (Thermo Scientific) according to manufacturer's protocol. Efficiency and purity of produced nanobodies was assessed by stain free SDS-PAGE (Biorad) as shown in FIG. 14.

### Example 17

1B10×CD3 Facilitates Immune Synapse Formation Between PBMCs and H1299 Stably Expressing HLA-A2/YLEYRQVPG.

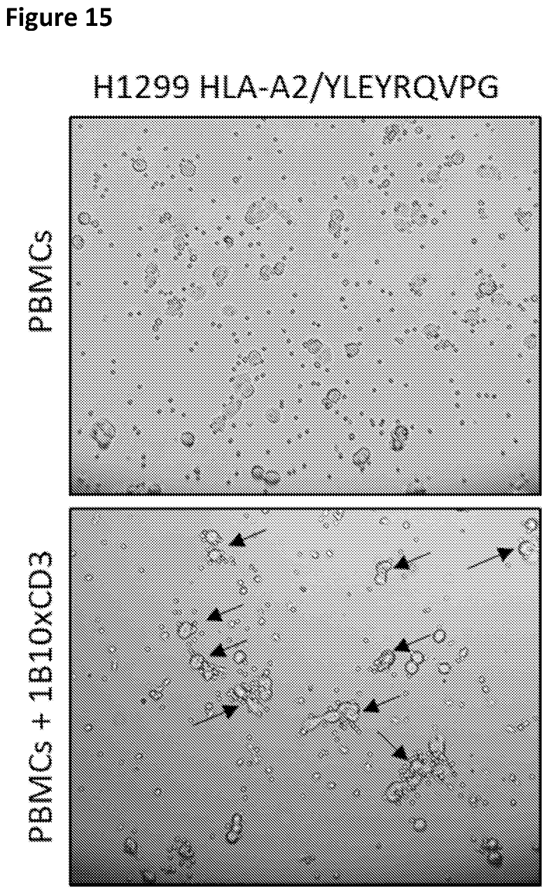

H1299 cells stably expressing HLA-A2/YLEYRQVPG were seeded at density of 50,000 cells per well and allowed to adhere for 16 hours. Next the culture medium was refreshed. PBMCs were added at target to effector ratio of 1:16, whereas bispecific nanobody molecule 1B10×CD3 was added at a final concentration of 100 ng/ml. Co-cultures were incubated for 24 hours at 37° C. Phase contrast images were taken in order to assess the effects of 1B10×CD3 on the interactions of PBMC and H1299 cells stably expressing HLA-A2/YLEYRQVPG. Interactions between effector cells (i.e. PBMCs) and target cells (i.e. H1299 transfectants) are indicated with black arrows (FIG. 15). Bispecific nanobody 1B10×CD3 facilitated formation of immune synapses between effector cells and target cells.

### Example 18

9A7×CD3 Leads to Specific T Cell Activation Resulting in Target Cells Number Reduction.

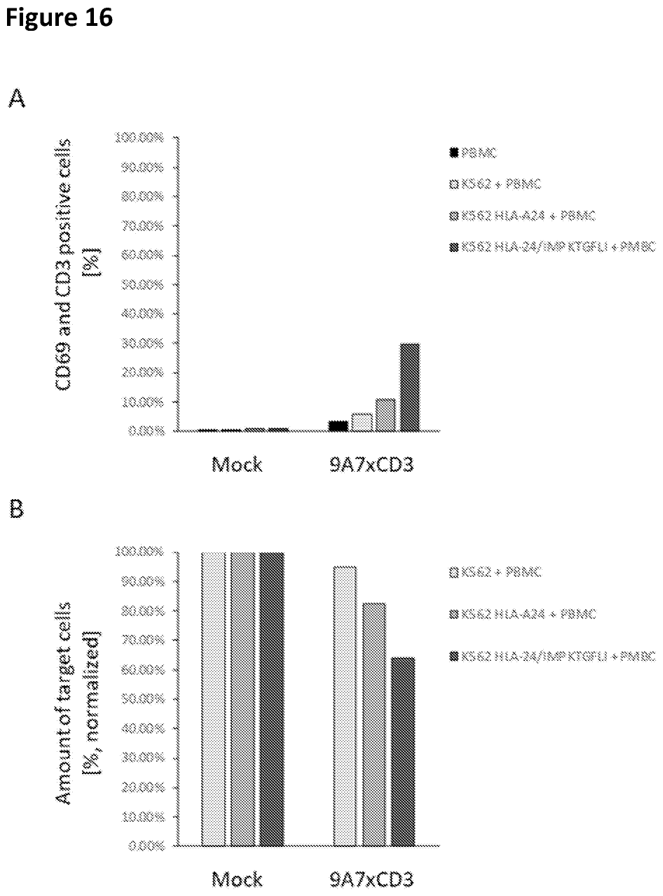

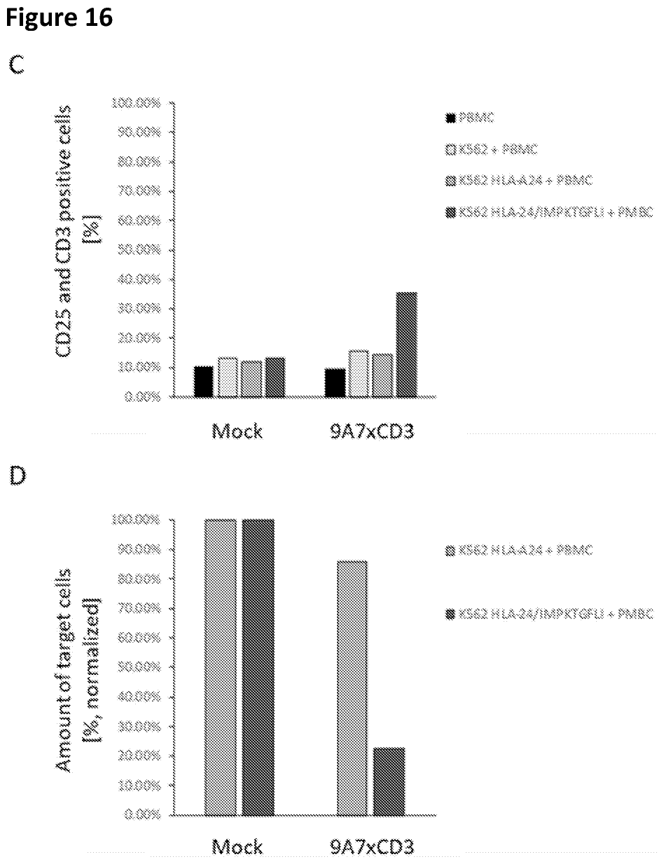

100,000 K562 cells, K562 cells stably expressing HLA-A*2402 (i.e. K562 HLA-A24) or K562 cells stably expressing HLA-A*2402/IMPKTGFLI (K562 HLA-A24/IMPKTGFLI) were seeded per well. Next day PBMCs were added at effector to target ratio of 8:1. 9A7×CD3 (SEQ ID NO: 65) was added to respective co-cultures at 50 ng/ml. Cells were incubated for 24 or 72 hours at 37° C. A flow cytometric analysis was performed after 24 hours of incubation in order to detect expression of T-cell early activation marker CD69. Results plotted as % of CD3 positive cells expressing CD69 are shown in FIG. 16A. After 24 hours of incubation expression of early activation marker CD69 was increased on surface of T cells only when PBMCs were co-cultured with K652 HLA-A24/IMPKTGFLI cells in presence of 9A7×CD3 BiTE, identified in this flow cytometric experiment by size, demonstrated a decrease in percentage of K562 HLA-A24/IMPKTGFLI cells (FIG. 16B). After 72 hours of incubation, an increase in expression of late T cell activation marker CD25 was observed (FIG. 16C). Measurement of percentage of target cells present after 72 hours of co-culture with PBMCs in presence of 9A7×CD3 BiTE showed a 80% decrease of K562 HLA-A24/IMPKTGFLI expressing cells (FIG. 16D).

### Example 19

14.1 9A7×CD3 Leads to Specific T Cell Activation Resulting in Reduction of Native Target Cells Number

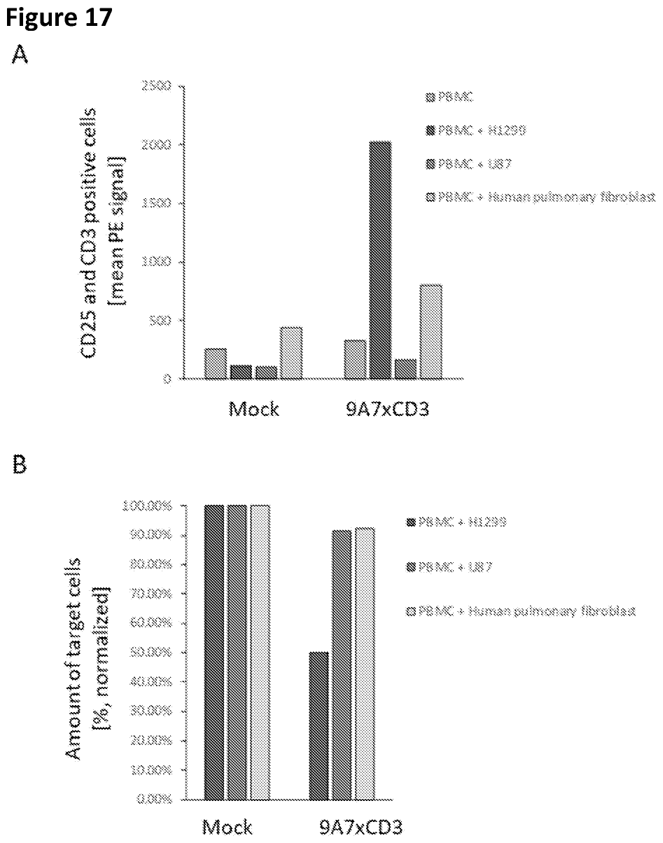

Here the H1299 cells were used as target cells due to expression of both MAGE-A and HLA-A*2402, whereas U87 cells and HPF cells were used as control cell lines. Human non-small cell lung carcinoma H1299 cells (MAGE-A and HLA-A*2402 positive), human U87 glioblastoma cells (MAGE-A positive, HLA-A*2402 negative) and human pulmonary fibroblasts (HLA-A*2402 positive; HPF) were seeded at a density of 20,000 cells per well and allowed to adhere for 16 hours. Next, medium was refreshed and PBMCs were added at effector to target ratio of 8:1. Cells were co-cultured for 72 hours at 37° C. in presence or absence of 9A7×CD3 (50 ng/ml). Flow cytometry was used to assess expression of late T cell activation marker and reduction in percentage of target cells present in co-culture. PMBCs co-cultured with H1299 and incubated with 9A7×CD3 (SEQ ID NO: 65) show a marked increase in expression levels of T cell activation marker CD25 (FIG. 17A). Moreover, a 50% decrease in percentage of H1299 cells present in co-culture was observed after 72 hours of PBMC incubation with target cells in presence of 9A7×CD3 (FIG. 17B). This decrease in percentage of target cells was not observed neither in case of U87 nor HPF. This result shows 9A7×CD3 BiTE efficiently directs and activates effector cells leading to eradication of MAGE-A and HLA-A*2402 positive target cells.

19.2 T Cell Activation by 9A7×CD3 is Concentration Dependent

A panel of following cell lines was used: human non-small cell lung carcinoma H1299 cells (MAGE-A and HLA-A*2402 positive), glioblastoma U118 cells (MAGE-A and HLA-A*2402 positive), chronic myelogenous leukemia K562 cells stably expressing HLA-A*2402/IMPKTGFLI and chronic myelogenous leukemia K652 cells stably expressing HLA-A*2402. Cells were seeded at density of 35′000 cells per well and allowed to adhere for 16 hours. Next, medium was refreshed and PBMCs were added at effector to target ratio of 8:1. Cells were co-cultured for 72 hours at 37° C. in presence or absence of 9A7×CD3 (50 ng/ml). Flow cytometry was used to asses expression of early T cell activation marker CD69 (FIG. 17C). Co-culturing of MAGE-A and HLA-A*2402 positive cells with PBMCs in presence of 9A7×CD3 (SEQ ID NO: 65) resulted in PBMC activation. The level of T cell activation differed depending on used target cell line. Co-culturing of PBMCs with H1299 cells resulted in similar level of PBMC activation irrespective of 9A7×CD3 concentration used. High expression level of CD69 was observed already in conditions treated with only 3.1 ng/ml 9A7×CD3. A 9A7×CD3 dose dependent increase of effector cells activation was observed in samples in which PBMCs were co-cultured with K562 HLA-A24/IMPKTGFLI. Effector cell activation was lowest when PBMCs were co-cultured with U118 cells in presence of 50-200 ng/ml 9A7×CD3. In case of PBMC and K562 HLA-A*2402 co-cultures (negative control setting) activation of effector cells was not observed at any of tested 9A7×CD3 concentrations. The activation of effector cells facilitated by 9A7×CD3 is driven by the specificity of the said molecule of the disclosure to HLA/MAGE complex (here the HLA-A24/IMPKTGFLI).

### Example 20

T Cell Activation by 9A7×CD3 is HLA-A*2402/IMPKTGFLI Specific and Leads to Target Cell Eradication

K652 cells stably expressing HLA-A*2402 (K562 HLA-A24) and K652 cells stably expressing HLA-A*2402/IMPKTGFLI (K562 HLA-A24/IMPKTGFLI) were seeded at amount of 100,000 cells per well, in presence or absence of PBMCs. In case of co-cultures effector to target ratio of 8:1 was used. Cells were incubated in presence or absence of 9A7×CD3 (50 ng/ml) for 72 hours. Phase contrast images were taken at the end of the incubation period. In co-cultures of K562 HLA-A24/IMPKTGFLI cells and PBMCs incubated in presence of 9A7×CD3 (SEQ ID NO: 65) reduced number of target cells was observed, as well as increased number of target-effector cell interactions (indicated by black arrows) (FIG. 18). In co-cultures of K562 HLA-A24 cells and PBMCs incubated in absence of 9A7×CD3 growth inhibition of K562 HLA-A24 cells was observed (FIG. 18). No reduction in target cell number or presence of effector cell-K562 HLA-A24 interactions were observed.

### Example 21

Interaction of Immune Cells with Cells Natively Expressing the Target HLA/MAGE Complex is Facilitated by 9A7×CD3

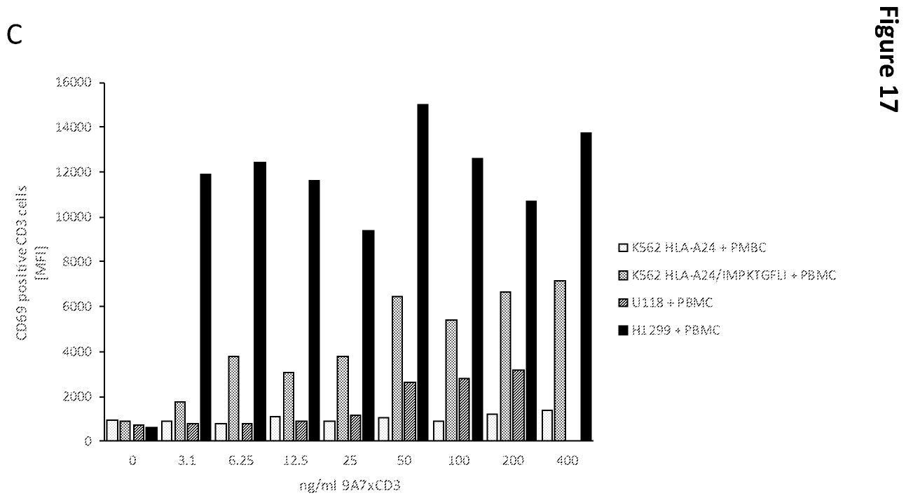

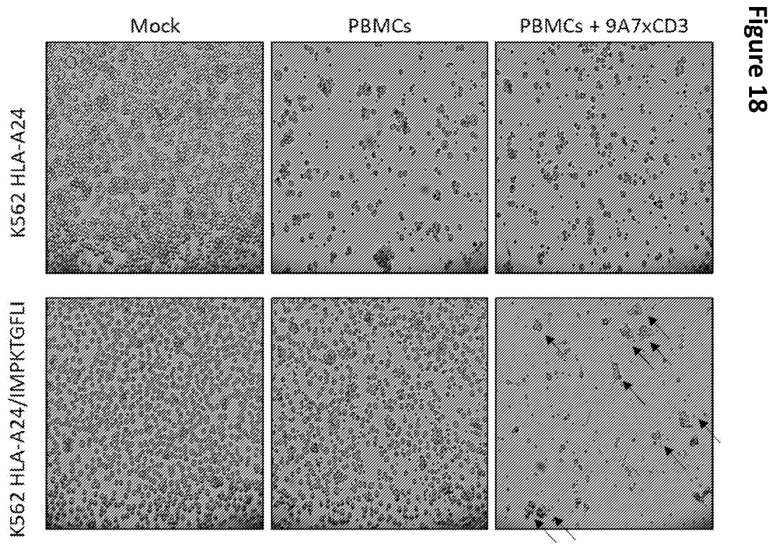

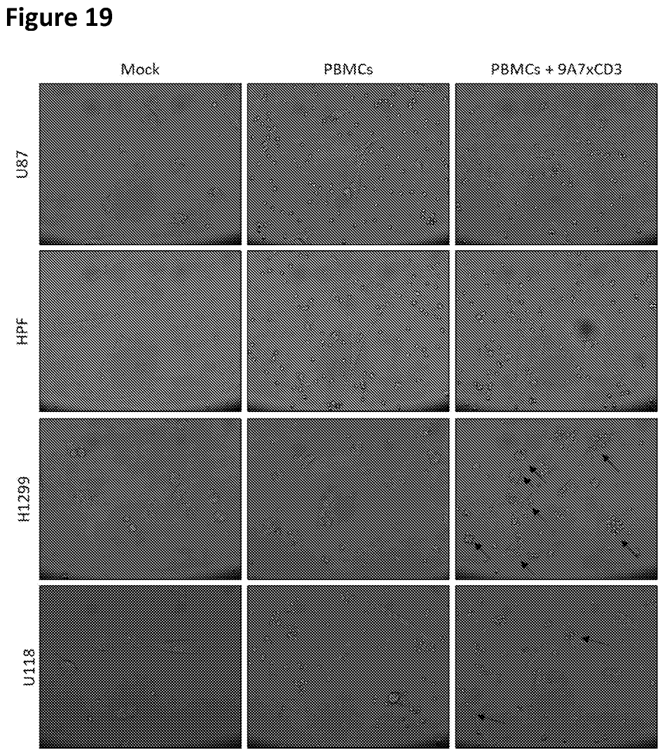

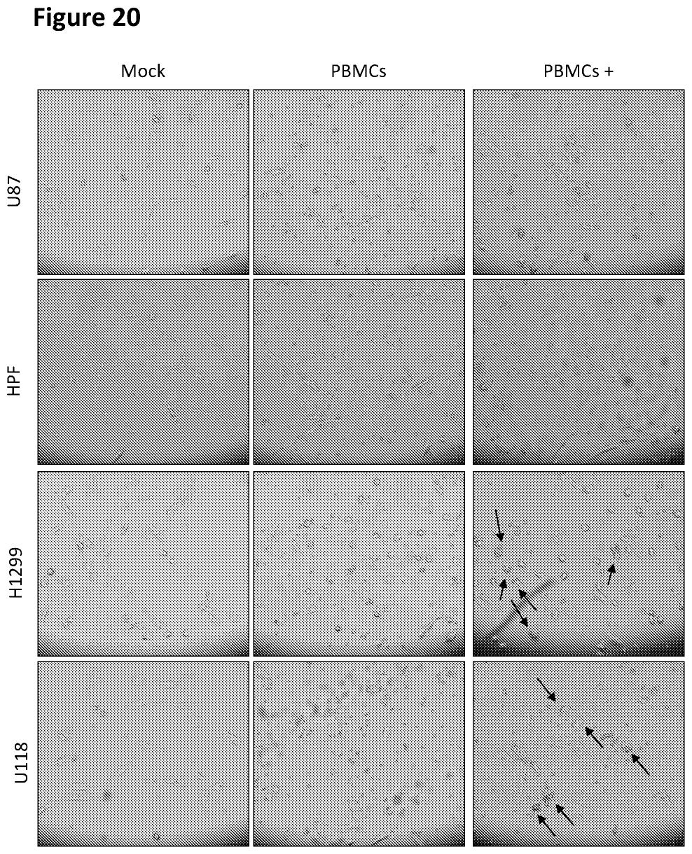

A panel of following cell lines was used: human non-small cell lung carcinoma H1299 cells (MAGE-A and HLA-A*2402 positive), human glioblastoma U118 cells (MAGE-A and HLA-A*2402 positive), human U87 glioblastoma cells (MAGE-A positive, HLA-A*2402 negative) and human pulmonary fibroblasts (HLA-A*2402 positive; HPF). Cells were seeded at a density of 50,000 cells per well and allowed to adhere for 16 hours. Next, medium was refreshed and PBMCs were added at effector to target ratio of 8:1. Cells were co-cultured for 24 hours at 37° C. in presence or absence of 9A7×CD3 (50 ng/ml). Phase contrast pictures were taken after 4 hours and after 24 hours incubation. As shown in FIGS. 19 and 20, 9A7×CD3 (SEQ ID NO: 65) induced formation of immune synapse between effector cells and H1299, and effector cells and U118 cells (indicated by black arrows) which were observed both after 4 hours (FIG. 19) and 24 hours (FIG. 20) incubation. Example 10.

Specific binding of phage display selected Fab fragments to HLA-A2/mMA complexes. Upon affinity driven phage display selection specific binders were eluted and obtained clones were expressed in bacteria. The periplasmic fractions were isolated and diluted 1:5. Neutravidin (at 2 μg/ml) plates were coated with 10 nM HLA-A2/mMA peptide. The binding of expressed Fab was detected upon incubation with detection antibodies: mouse anti-c-myc (1:1000) and anti-mouse IgG-HRP (1:5000). As a positive control AH5 Fab (produced from pCES vector) and AH5 monoclonal IgG were used. Binding of produced Fab clones was assessed in parallel on plates coated with HLA-A2/mMA peptide complex and plates coated with control HLA-A2/MA3 peptide complex. Only Fab clones which showed binding to HLA-A2/mMA peptide complex (upper table in FIG. 21) and not to HLA-A2/MA3 peptide complex (bottom table in FIG. 21) were considered to have the desired specificity towards HLA-A2 presenting the multi MAGE peptide (YLEYRQVPG). Clones that showed binding to both types of complexes were considered to be recognizing the HLA-A2 part of the complexes and lack the desired fine specificity. Table 2 provides overview of VH sequences specifically binding MAGE derived peptides in complex with HLA-A0201. SEQ ID NO 3 till SEQ ID NO 46 represent VH sequence of Fab specifically binding HLA-A2/mMA complex.

TABLE 2

| SEQ ID NO | Description |
|---|---|
| 1 | Vh 4A6 |
| 2 | Vh A09 |
| 3 | MP08A03 |
| 4 | MP08A08 |
| 5 | MP08A09 |
| 6 | MP08B02 |
| 7 | MP08B06 |
| 8 | MP08C01 |
| 9 | MP08C03 |
| 10 | MP08C10 |
| 11 | MP08D02 |
| 12 | MP08D03 |
| 13 | MP08D04 |
| 14 | MP08D07 |
| 15 | MP08D10 |
| 16 | MP08E05 |
| 17 | MP08E06 |
| 18 | MP08E10 |
| 19 | MP08E11 |
| 20 | MP08F02 |
| 21 | MP08F03 |
| 22 | MP08F04 |
| 23 | MP08F05 |
| 24 | MP08F06 |
| 25 | MP08F08 |
| 26 | MP08F09 |
| 27 | MP08G02 |
| 28 | MP08G04 |
| 29 | MP08H01 |
| 30 | MP08H02 |
| 31 | MP08H05 |
| 32 | MP08H09 |
| 33 | MP08H10 |
| 34 | MP09A10 |
| 35 | MP09B10 |
| 36 | MP09C01 |
| 37 | MP09C02 |
| 38 | MP09C03 |
| 39 | MP09C04 |
| 40 | MP09D03 |
| 41 | MP09D09 |
| 42 | MP09E01 |
| 43 | MP09G02 |
| 44 | MP09G03 |
| 45 | MP09G05 |
| 46 | MP09H01 |

Sequence Identifier Numbers (SEQ ID NOs):

SEQ ID NO: 1. Amino-acid sequence Vh of 4A6 IgG
EVQLVQSGAEVKKPGSSVKVSCKASGGTFSSYAISWVRQAPGQGLEWM
GGIIPIFGTADYAQKFQGRATITADESTSTAYMELSSLRSEDTAVYYCAR
DYDFWSGYYAGDVWGQGTTVTVSS
SEQ ID NO: 2. Amin-oacid sequence Vh of A09 IgG
QVQLVESGGGVVQPGRSLRLSCAASGFTFSTFPMHWVRQAPGKGLEWV
AVIDYEGINKYYADSVKGRFTISRDNSKNTLYLQMNSLRAEDTAVYYCAG
GSYYVPDYWGQGTLVTVSS
SEQ ID NO: 3
QLQLQESGGGWQPGRSLRLSCAASGFTFSSFPMMWIRQAPGKGLEWVASISYDGSNKYYADS
VKGRFTISRDNSKNTLYLQMNSLRAEDTAVYYCAGGSYYVPDYWGQGTLVTVSS
SEQ ID NO: 4
QLQLQESGGGWQPGRSLRLSCAASGFTFSRNqMWWVRQAPGKGLEWVAVISIDQSVKYYAD
SVKGRFTISRDNSKNTLYLQMNSLRAEDTAVYYCAGGSYYVPDYWGQGTLVTVSS
SEQ ID NO: 5
QLQLQESGGGWQPGRSLRLSCAASGFTFSTFPMHWVRQAPGKGLEWVAVIDYEGINKYYADS
VKGRFTISRDNSKNTLYLQMNSLRAEDTAVYYCAGGSYYVPDYWGQGTLVTVSS
SEQ ID NO: 6
QLQLQESGGGWQPGRSLRLSCAASGFTFSESAMHWVRQAPGKGLEWVAAISYDGSNKYYAD
SVKGRFTISRDNSKNTLYLQMNSLRAEDTAVYYCAGGSYYVPDYWGQGTLVTVSS
SEQ ID NO: 7
QLQLQESGGGWQPGRSLRLSCAASGFTFSVFAMQWVRQAPGKGLEWVAAISYDGDNKYYAD
SVKGRFTISRDNSKNTLYLQMNSLRAEDTAVYYCAGGSYYVPDYWGQGTLVTVSS
SEQ ID NO: 8
QLQLQESGGGWQPGRSLRLSCAASGFTFSERQMWVWRQAPGKGLEWVAVISNDTSSKYYAD
SVKGRFTISRDNSKNTLYLQMNSLRAEDTAVYYCAGGSYYVPDYWGQGTLVTVSS
SEQ ID NO: 9
QLQLQESGGGWQPGRSLRLSCAASGFTFSERqMWWVRQAPGKGLEWVAVISHDGSTKYYAD
SVKGRFTISRDNSKNTLYLQMNSLRAEDTAVYYCAGGSYYVPDYWGQGTLVTVSS
SEQ ID NO: 10
QLQLQESGGGWQPGRSLRLSCAASGFTFSSRQMWWVRPAPGKGLEWVAVISHDASAKYYAD
SVKGRFTISRDNSKNTLYLQMNSLRAEDTAVYYCAGGSYYVPDYWGQGTLVTVSS
SEQ ID NO: 11
QLQLQESGGGWQPGRSLRLSCAASGFTFSVISMQWVRQAPGKGLEWVASISYDGSNKYYADS
VKGRFTISRDNSKNTLYLQMNSLRAEDTAVYYCAGGSYYVPDYWGQGTLVTVSS
SEQ ID NO: 12
QLQLQESGGGWQPGRSLRLSCAASGFTFSTFPMHWVRQAPGKGLEWVAAISYAGSNKYYAD
SVKGRFTISRDNSKNTLYLQMNSLRAEDTAVYYCAGGSYYVPDYWGQGTLVTVSS
SEQ ID NO: 13
QLQLQESGGGWQPGRSLRLSCAASGFTFSTLPMHWVRQAPGKGLEWVAVISYNGENKYYAD
SVKGRFTISRDNSKNTLYLQMNSLRAEDTAVYYCAGGSYYVPDYWGQGTLVTVSS
SEQ ID NO: 14
QLQLQESGGGWQPGRSLRLSCAASGFTFSTLPMHWVRQAPGKGLEWVAVISYDGSNKYYAD
SVKGRFTISRDNSKNTLYLQMNSLRAEDTAVYYCAGGSYYVPDYWGQGTLVTVSS
SEQ ID NO: 15
QLQLQESGGGWQPGRSLRLSCAASGFTFSERQMWWVRQAPGKGLEWVAVISNDSSQKYYA
DSVKGRFTISRDNSKNTLYLQMNSLRAEDTAVYYCAGGSYYVPDYWGQGTLVTVSS
SEQ ID NO: 16
QLQLQESGGGWQPGRSLRLSCAASGFTFSTMSMQWVRQAPGKGLEWVASISYDGSNKYYAD
SVKGRFTISRDNSKNTLYLQMNSLRAEDTAVYYCAGGSYYVPDYWGQGTLVTVSS
SEQ ID NO: 17
QLQLQESGGGWQPGRSLRLSCAASGFTFSTLSMGWVRQAPGKGLEWVAWISYDGSNKYYAD
SVKGRFTISRDNSKNTLYLQMNSLRAEDTAVYYCAGGSYYVPDYWGQGTLVTVSS
SEQ ID NO: 18
QLQLQESGGGWQPGRSLRLSCAASGFTFSTSAMQWVRQAPGKGLEWVAVIGYDGANKYYAD
SVKGRFTISRDNSKNTLYLQMNSLRAEDTAVYYCAGGSYYVPDYWGQGTLVTVSS
SEQ ID NO: 19
QLQLQESGGGWQPGRSLRLSCAASGFTFSTLPMHWVRQAPGKGLEWVAVISYDGSNKYYAD
SVKGRFTISRDNSKNTLYLQMNSLRAEDTAVYYCAGGSYYVPDYWGQGTLVTVSS
SEQ ID NO: 20
QLQLQESGGGWQPGRSLRLSCAASGFTFSSYAMHWVRQAPGKGLEWVAAISYDGRNKYYAD
SVKGRFTISRDNSKNTLYLQMNSLRAEDTAVYYCAGGSYYVPDYWGQGTLVTVSS
SEQ ID NO: 21
QLQLQESGGGWQPGRSLRLSCAASGFTFSAGqMWWVRQAPGKGLEWVAVISHDESNKYYAD
SVKGRFTISRDNSKNTLYLQMNSLRAEDTAVYYCAGGSYYVPDYWGQGTLVTVSS
SEQ ID NO: 22
QLQLQESGGGWQPGRSLRLSCAASGFTFSTYPMHWVRQAPGKGLEWVAVISYTGINKYYADS
VKGRFTISRDNSKNTLYLQMNSLRAEDTAVYYCAGGSYYVPDYWGQGTLVTVSS
SEQ ID NO: 23
QLQLQESGGGWQPGRSLRLSCAASGFTFSSRQMWVWRQAPGKGLEWVAVISHDASAKYYAD
SVKGRFTISRDNSKNTLYLQMNSLRAEDTAVYYCAGGSYYVPDYWGQGTLVTVSS
SEQ ID NO: 24
QLQLQESGGGWQPGRSLRLSCAASGFTFSESAMHWVRQAPGKGLEWVAVISYSGMNKYYAD
SVKGRFTISRDNSKNTLYLQMNSLRAEDTAVYYCAGGSYYVPDYWGQGTLVTVSS
SEQ ID NO: 25
QLQLQESGGGWQPGRSLRLSCAASGFTFSAGqMWWVRQAPGKGLEWVAVISHDESNKYYAD
SVKGRFTISRDNSKNTLYLQMNSLRAEDTAVYYCAGGSYYVPDYWGQGTLVTVSS
SEQ ID NO: 26
QLQLQESGGGWQPGRSLRLSCAASGFTFSESAMGWVRQAPGKGLEWVAWIGYDGQNKYYA
DSVKGRFTISRDNSKNTLYLQMNSLRAEDTAVYYCAGGSYYVPDYWGQGTLVTVSS
SEQ ID NO: 27
QLQLQESGGGWQPGRSLRLSCAASGFTFSSqTMQWVRQAPGKGLEWVASISYDGENKYYAD
SVKGRFTISRDNSKNTLYLQMNSLRAEDTAVYYCAGGSYYVPDYWGQGTLVTVSS
SEQ ID NO: 28
QLQLQESGGGWQPGRSLRLSCAASGFTFSTLPMHWVRQAPGKGLEWVAVISYNGENKYYAD
SVKGRFTISRDNSKNTLYLQMNSLRAEDTAVYYCAGGSYYVPDYWGQGTLVTVSS
SEQ ID NO: 29
QLQLQESGGGWQPGRSLRLSCAASGFTFSVQSMLWVRQAPGKGLEWVASIGYDGVNKYYAD
SVKGRFTISRDNSKNTLYLQMNSLRAEDTAVYYCAGGSYYVPDYWGQGTLVTVSS
SEQ ID NO: 30
QLQLQESGGGWQPGRSLRLSCAASGFTFSRNqMWWVRQAPGKGLEWVAVISIDQSVKYYAD
SVKGRFTISRDNSKNTLYLQMNSLRAEDTAVYYCAGGSYYVPDYWGQGTLVTVSS
SEQ ID NO: 31
QLQLQESGGGWQPGRSLRLSCAASGFTFSSFPMQWVRQAPGKGLEWVASIAYDGSNKYYAD
SVKGRFTISRDNSKNTLYLQMNSLRAEDTAVYYCAGGSYYVPDYWGQGTLVTVSS
SEQ ID NO: 32
QLQLQESGGGWQPGRSLRLSCAASGFTFSMFAMHWVRQAPGKGLEWVAAISIDGSGKYYAD
SVKGRFTISRDNSKNTLYLQMNSLRAEDTAVYYCAGGSYYVPDYWGQGTLVTVSS
SEQ ID NO: 33
QLQLQESGGGWQPGRSLRLSCAASGFTFSESPMFWVRQAPGKGLEWVAVISYTGYNKYYAD
SVKGRFTISRDNSKNTLYLQMNSLRAEDTAVYYCAGGSYYVPDYWGQGTLVTVSS
SEQ ID NO: 34
QLQLQESGGGWQPGRSLRLSCAASGFTFSRHRMFWVRQAPGKGLEWVAGIGYWGWNKYYA
DSVKGRFTISRDNSKNTLYLQMNSLRAEDTAVYYCAGGSYYVPDYWGQGTLVTVSS
SEQ ID NO: 35
QLQLQESGGGWQPGRSLRLSCAASGFTFSWRQMWWVRQAPGKGLEWVAVISHDGSGKYYA
DSVKGRFTISRDNSKNTLYLQMNSLRAEDTAVYYCAGGSYYVPDYWGQGTLVTVSS
SEQ ID NO: 36
QLQLQESGGGWQPGRSLRLSCAASGFTFSSSqMWVWRQAPGKGLEWVAVISHDTSSKYYAD
SVKGRFTISRDNSKNTLYLQMNSLRAEDTAVYYCAGGSYYVPDYWGQGTLVTVSS
SEQ ID NO: 37
QLQLQESGGGWQPGRSLRLSCAASGFTFSRQQMWWVRQAPGKGLEWVAVISLDPSIKYYAD
SVKGRFTISRDNSKNTLYLQMNSLRAEDTAVYYCAGGSYYVPDYWGQGTLVTVSS
SEQ ID NO: 38
QLQLQESGGGWQPGRSLRLSCAASGFTFSMFAMHWVRQAPGKGLEWVAAISIDGSGKYYAD
SVKGRFTISRDNSKNTLYLQMNSLRAEDTAVYYCAGGSYYVPDYWGQGTLVTVSS
SEQ ID NO: 39
QLQLQESGGGWQPGRSLRLSCAASGFTFSSIPMFWVRQAPGKGLEWVASISYNGENKYYADS
VKGRFTISRDNSKNTLYLQMNSLRAEDTAVYYCAGGSYYVPDYWGQGTLVTVSS
SEQ ID NO: 40
QLQLQESGGGWQPGRSLRLSCAASGFTFSESSMQWVRQAPGKGLEWVASIGYDGqNKYYAD
SVKGRFTISRDNSKNTLYLQMNSLRAEDTAVYYCAGGSYYVPDYWGQGTLVTVSS
SEQ ID NO: 41
QLQLQESGGGWQPGRSLRLSCAASGFTFSVQSMQWVRQAPGKGLEWVAAIGYDGENKYYAD
SVKGRFTISRDNSKNTLYLQMNSLRAEDTAVYYCAGGSYYVPDYWGQGTLVTVSS
SEQ ID NO: 42
QLQLQESGGGWQPGRSLRLSCAASGFTFSESAMHWVRQAPGKGLEWVAAISYDGSNKYYAD
SVKGRFTISRDNSKNTLYLQMNSLRAEDTAVYYCAGGSYYVPDYWGQGTLVTVSS
SEQ ID NO: 43
QLQLQESGGGWQPGRSLRLSCAASGFTFSERqMWWVRQAPGKGLEWVAVISHDGSTKYYAD
SVKGRFTISRDNSKNTLYLQMNSLRAEDTAVYYCAGGSYYVPDYWGQGTLVTVSS
SEQ ID NO: 44
QLQLQESGGGWQPGRSLRLSCAASGFTFSSFAMHWVRQAPGKGLEWVAVISYDGSNKYYAD
SVKGRFTISRDNSKNTLYLQMNSLRAEDTAVYYCAGGSYYVPDYWGQGTLVTVSS
SEQ ID NO: 45
QLQLQESGGGWQPGRSLRLSCAASGFTFSERqMWWVRQAPGKGLEWVAVISHDGSTKYYAD
SVKGRFTISRDNSKNTLYLQMNSLRAEDTAVYYCAGGSYYVPDYWGQGTLVTVSS
SEQ ID NO: 46
QLQLQESGGGWQPGRSLRLSCAASGFTFSSLPMHWVRQAPGKGLEWVAAISYDGSNKYYAD
SVKGRFTISRDNSKNTLYLQMNSLRAEDTAVYYCAGGSYYVPDYWGQGTLVTVSS
SEQ ID NO: 47
EVQLVESGGGLVQHGGSLRLSCASSGTFFSINVMGWYRQAPGKQRDLF
ADISRSGNTNYADSVNGRFTISRDIAKNTVYLQMNSLKPEDTGVYYCYAS
ADSHGRRVLTPYWGEGTQVTVSS
SEQ ID NO: 48
EVQLVESGGGLVQAGGSLRLSCTASGFTLDYKALVWFRQAPGKERERV
SCISGGGGSTYYADSVKGRFTISRDNTKNTVYLQMNSLKPEDTAVYTCA
APQSFACTATLKYWGQGTQVTVSS
SEQ ID NO: 49
EVQLVESGGGLVHPGGSLRLSCAASGFTFSTYSMSWVRQAPGKGLEWV
SSITPLGLSTKYGESVKGRFTISRDNAKKMLYLQMNSLKREDTAVYYCAK
YPPGTQSITAKVRYDYRGQGTQVTVSS
SEQ ID NO: 50
EVQLVESGGGLVHPGGSLRLSCAASGFTFSTYSMSWVRQAPGKGLEWV
SSITPLGLSTKYGESVKGRFTISRDNAKKMLYLQMNSLKREDTAVYYCAK
YPPGTQSITAKVRYDYRGQGTQVTVSS
SEQ ID NO: 51
EVQQMEFGGGLVQNRGSRKLSCQASRMLFKVNTMGWYRQGPGKQSAW
VADTTEGGSIKYADSVKGRFTISRDNAKTTAYLQKNSLKPEDTTLYFCNA
IDRVNSQKWGQGTQVTVST
SEQ ID NO: 52
EVQLVESGGGLVQPGGSLKLSCQASRMLFKVNTMGWYRQGPGKQRELV
ADITEGGSIKYADSVKGRFTISRDNAKTTVYLQMNSLKPEDTAVYFCNAI
DRVNSQYWGQGTQVTVSS
SEQ ID NO: 53
EVQLVESGGGLVQPGGSLKLSCQASRMLFKVNTMGWYRQGPGKQRELV
ADITEGGSIKYADSVKGRFTISRDNAKTTVYLQMNSLKPEDTAVYFCNAI
DRVNSQYWGQGTQVTVSS
SEQ ID NO: 54
EVQLVESGGTLVQPGGSLRLSCEASGFSFSTTHMSWVRQAPGKGLEWV
ARISSDGSRTTYADSVKGRFTISRDNAKNALYLQMNNLTFEDAAVYFCST
TITERRGRGTQVTVSS
SEQ ID NO: 55
EVQLVESGGGLVQHGGSLRLSCAASGTFFSINVMGWYRQAPGKQRDLV
ADISRTGNTNYADSVKGRFTISRDIAKNTVYLQMNSLKPEDTGVYYCYAS
AVSDGRRVLTPYWGEGTQVTVSS
SEQ ID NO: 56
EVQLVESGGGLVQAGGSLRLSCTASGFTLDYKALVWFRQAPGKERERV
SCISGGGGSTYYADSVKGRFTISRDNTKNTVYLQMNSLKPEDTAVYTCA
APQSFACTATLKYWGQGTQVTVSS
SEQ ID NO: 57
QVKLEESGGGLVQAGGLLRVSCTASGRTFDTMGWFRQAPGKEREFVAAVRWSSGNTLYGNTVKGRFTI
SRDTATNTVYLQMSSLKHEDTAVYYCAARWGGRGAADHWGQGTQVTVSS
SEQ ID NO: 58
EVQLVESGGGLVQPGGSLKLSCQASRMLFKVNTMGWYRQGPGKQRELVADITEGGSIKYADSVKGRFTI
SRDNAKTTVYLQMNSLKPEDTAVYFCNAIDRVNSQYWGQGTQVTVSSGGGGSQVKLEESGGGLVQAG
GLLRVSCTASGRTFDTMGWFRQAPGKEREFVAAVRWSSGNTLYGNTVKGRFTISRDTATNTVYLQMSS
LKHEDTAVYYCAARWGGRGAADHWGQGTQVTVSSHHHHHH
SEQ ID NO: 59
QSALTQPASVSGSPGQSITISCTGTSSDVGGYNYVSWYQQHPGKAPKLMIYDVSNRPSGVSNRFSGSKS
GNTASLTISGLQAEDEADYYCSSYTSSSTRVFGGGTKLTVL
SEQ ID NO: 60
NFMLTQPHSVSESPGKTVTISCTRSSGSIASYYVQWYQQRPGSSPTTVISEDNQRPSGVPDRFSGSIDSS
SNSASLTISGLKTEDEADYYCQSYDSSNVWVFGGGTKLTVL
SEQ ID NO: 61
QVQLQQPGAELVKPGASVKLSCKASGYTFTSFWMHWVKQRPGQGLEWIGEIDSSDSYTNYNQKFKGKA
TLTVDKSSSTAYMQLSSLTSDDSAVYYCARRVGRGYFDYWGQGTTLTVSS
SEQ ID NO: 62
DIVMTQSHKFMSTSVGDRVSITCKASQDVRTAVVWYQQKPGQSPKLLIYWASTRHTGVPDRFTGSGSGT
DYILTISSVQAEDLALYYCQQYDTTPWTFGGGTKVEIK
SEQ ID NO: 63
QSALTQPASVSGSPGQSITISCTGTSSDVGGYNYVSWYQQHPGKAPKLMIYDVSNRPSGVSNRFSGSKS
GNTASLTISGLQAEDEADYYCSSYTSSSTRVFGGGTKLTVLGGGGSGGGGSGGGGSEVQLVQSGAEVK
KPGSSVKVSCKASGGTFSSYAISWWRQAPGQGLEWMGGIIPIFGTADYAQKFQGRATITADESTSTAYME
LSSLRSEDTAVYYCARDYDFWSGYYAGDVWGQGTTVTVSSGGGGSDIKLQQSGAELARPGASVKMSC
KTSGYTFTRYTMHWWKQRPGQGLEWIGYINPSRGYTNYNQKFKDKATLTTDKSSSTAYMQLSSLTSEDS
AVYYCARYYDDHYCLDYWGQGTTLTVSSVEGGSGGSGGSGGSGGVDDIQLTQSPAIMSASPGEKVTMT
CRASSSVSYMNWYQQKSGTSPKRWIYDTSKVASGVPYRFSGSGSGTSYSLTISSMEAEDAATYYCQQW
SSNPLTFGAGTKLELKHHHHHH
SEQ ID NO: 64
NFMLTQPHSVSESPGKTVTISCTRSSGSIASYYVQWYQQRPGSSPTTVISEDNQRPSGVPDRFSGSIDSS
SNSASLTISGLKTEDEADYYCQSYDSSNVWVFGGGTKLTVLGGGGSGGGGSGGGGSQVQLVESGGGV
VQPGRSLRLSCAASGFTFSTFPMHWVRQAPGKGLEWVAVIDYEGINKYYADSVKGRFTISRDNSKNTLY
LQMNSLRAEDTAVYYCAGGSYYVPDYWGQGTLVTVSSGGGGSDIKLQQSGAELARPGASVKMSCKTS
GYTFTRYTMHWVKQRPGQGLEWIGYINPSRGYTNYNQKFKDKATLTTDKSSSTAYMQLSSLTSEDSAVY
YCARYYDDHYCLDYWGQGTTLTVSSVEGGSGGSGGSGGSGGVDDIQLTQSPAIMSASPGEKVTMTCR
ASSSVSYMNWYQQKSGTSPKRWIYDTSKVASGVPYRFSGSGSGTSYSLTISSMEAEDAATYYCQQWSS
NPLTFGAGTKLELKHHHHHH
SEQ ID NO: 65
DIVMTQSHKFMSTSVGDRVSITCKASQDVRTAVVWYQQKPGQSPKLLIYWASTRHTGVPDRFTGSGSGT
DYILTISSVQAEDLALYYCQQYDTTPWTFGGGTKVEIKGGGGSGGGGSGGGGSQVQLQQPGAELVKPG
ASVKLSCKASGYTFTSFWMHWVKQRPGQGLEWIGEIDSSDSYTNYNQKFKGKATLTVDKSSSTAYMQL
SSLTSDDSAVYYCARRVGRGYFDYWGQGTTLTVSSGGGGSDIKLQQSGAELARPGASVKMSCKTSGYT
FTRYTMHWVKQRPGQGLEWIGYINPSRGYTNYNQKFKDKATLTTDKSSSTAYMQLSSLTSEDSAVYYCA
RYYDDHYCLDYWGQGTTLTVSSVEGGSGGSGGSGGSGGVDDIQLTQSPAIMSASPGEKVTMTCRASS
SVSYMNWYQQKSGTSPKRWIYDTSKVASGVPYRFSGSGSGTSYSLTISSMEAEDAATYYCQQWSSNPL
TFGAGTKLELKHHHHHH
SEQ ID NO: 66
VH (specific to CD3)
DIKLQQSGAELARPGASVKMSCKTSGYTFTRYTMHWKQRPGQGLEWIGYINPSRGYTNYNQKFKDKA
TLTTDKSSSTAYMQLSSLTSEDSAVYYCARYYDDHYCLDYWGQGTTLTVSS

### Example 22

Flow Cytometric Alanine Scanning Assay Determined Fine Specificity of 9A7×CD3.

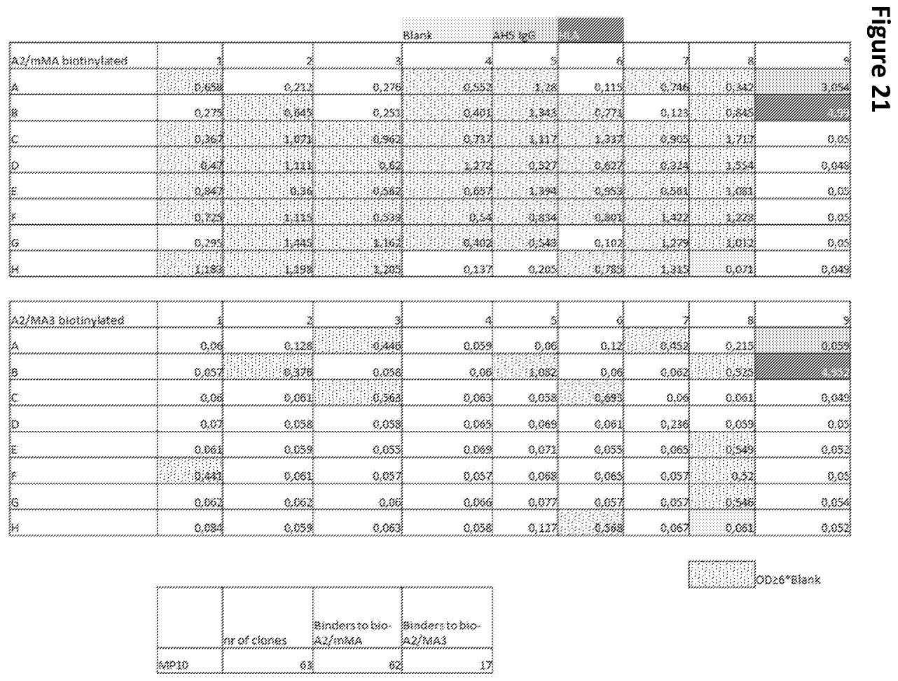

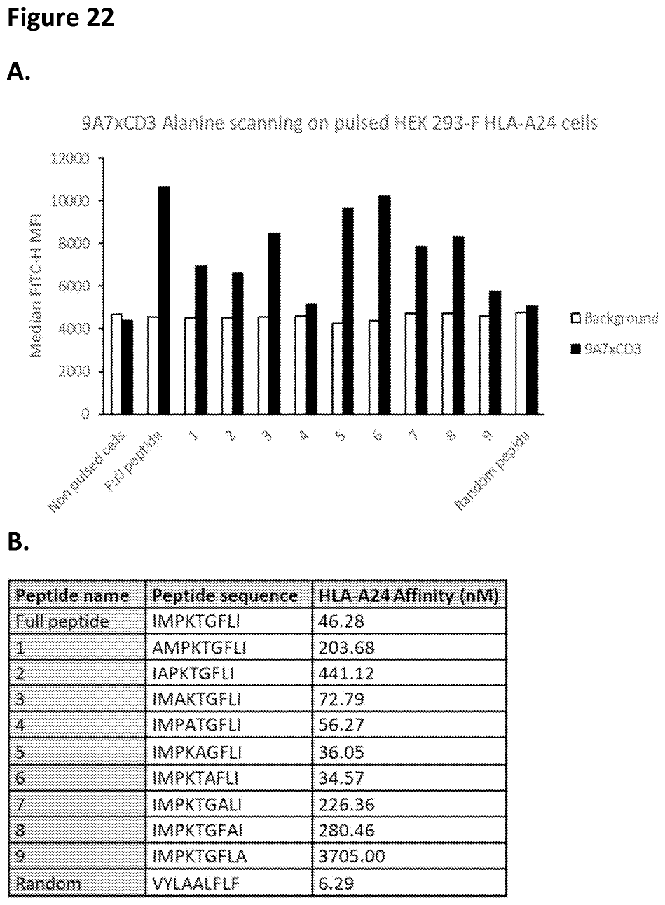

HEK 293-F cells were transfected 2 days prior to the alanine scanning to express HLA-*A2402 (HLA-A24) on the cell membrane. Fine specificity of the bispecific molecule was assessed by pulsing 200.000 HEK 293-F HLA-A24 cells overnight under serum free conditions with 100 μg/ml peptide variants as indicated in FIG. 22B. The amino acids of the used peptides were sequentially substituted for an alanine. Pulsed HEK 293-F cells were incubated with constant concentration of 12 μg/ml 9A7×CD3 (SEQ ID NO: 65). The binding of 9A7×CD3 (SEQ ID NO: 65) was detected upon incubation with anti-6×his-FITC antibody (Invitrogen, cat. no. MA1-81891). To determine the background signal, samples with only anti-6×his-FITC without 9A7×CD3 (SEQ ID NO: 65) staining were used. The obtained binding pattern presented in FIG. 22A showed individual amino acids contributions to the binding of 9A7×CD3 (SEQ ID NO: 65) to the HLA-A24/IMPKTGFLI complex. Amino acids at positions 1 (I), 2 (M), 3 (P), 7 (F) and 8 (L) are contributing to the binding of 9A7×CD3 (SEQ ID NO: 65) towards the HLA-A2/IMPKTGFLI complex. Essential amino acids in the peptide for the binding of 9A7×CD3 (SEQ ID NO: 65) towards the complex are at positions 4 (K) and 9 (I). Table presented in FIG. 22B lists amino acid sequences of all peptides used in this alanine scan experiment, as well as their predicted affinity to HLA-A24.

### Example 23 T Cell Activation

9A7×CD3 Induces T Cell Activation in the Presence of HLA-A*2402 and MAGE-A Expressing Cells

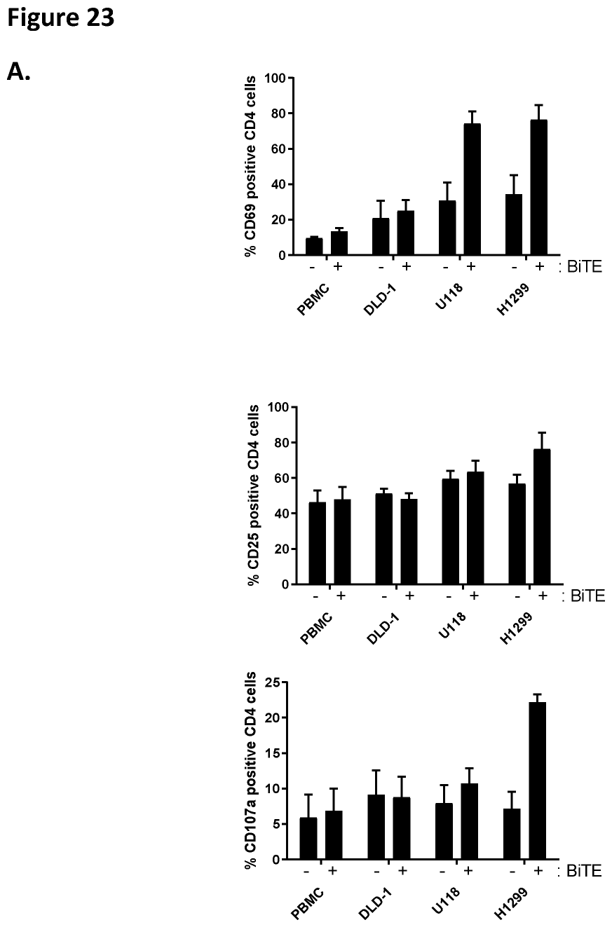

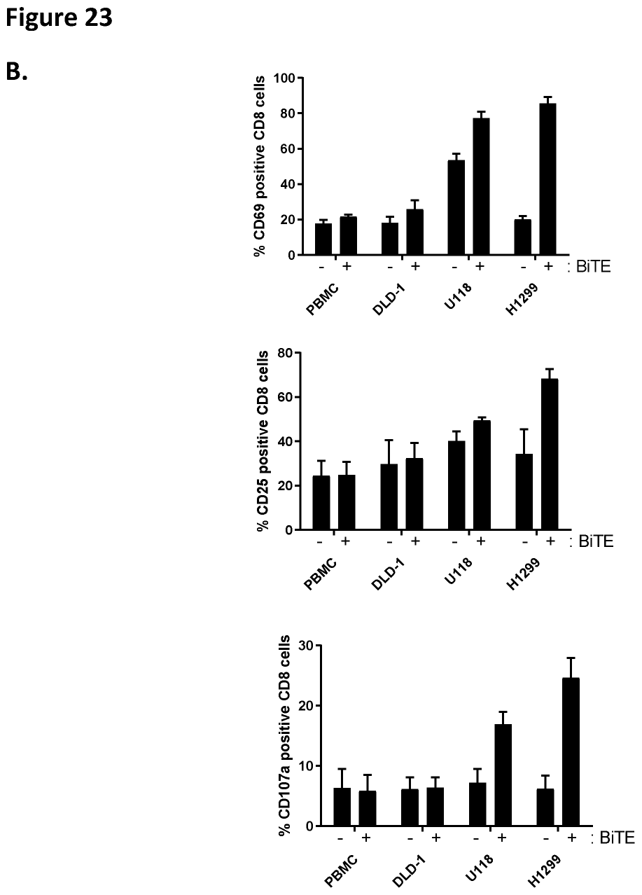

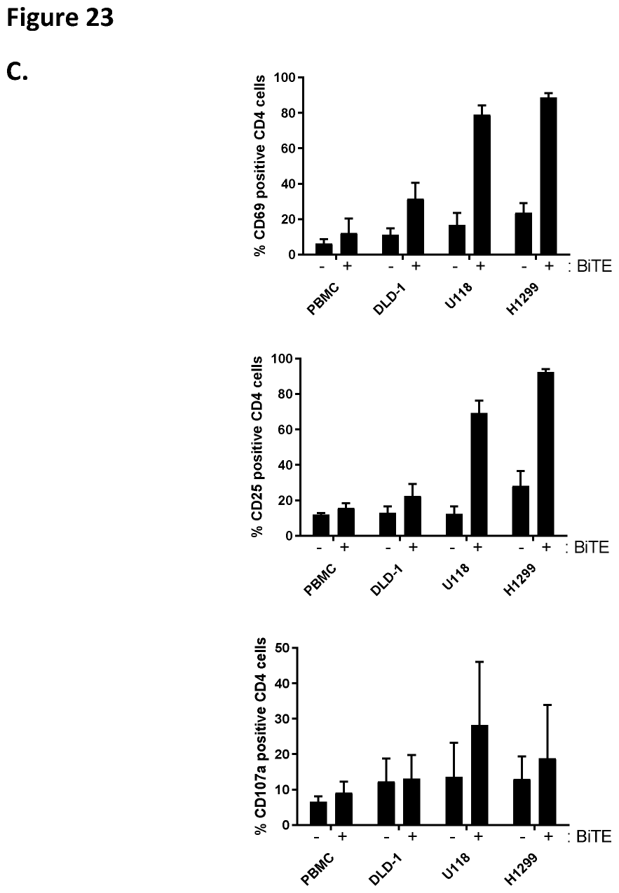

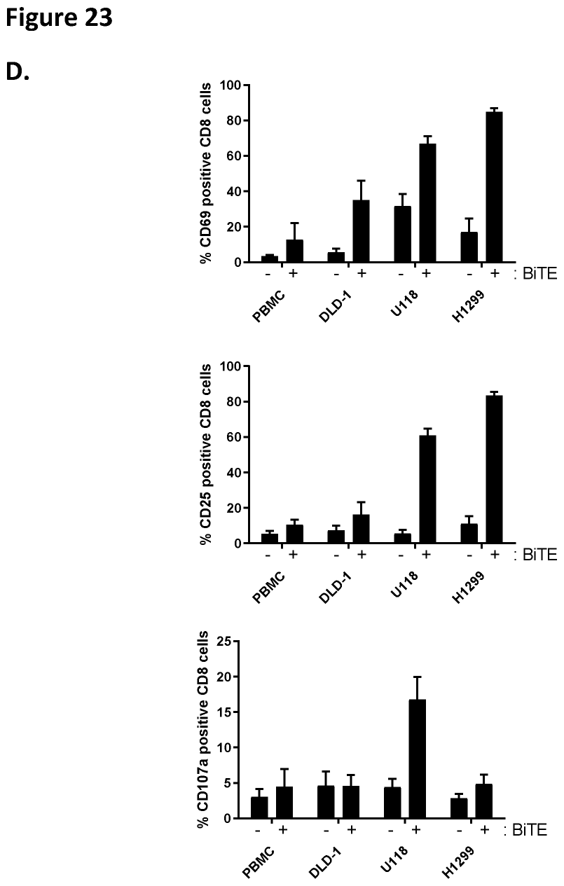

Non small cell lung carcinoma H1299 (HLA-A*2402 and MAGE-A positive), brain glioblastoma U118 (HLA-A*2402 and MAGE-A positive) as well as colon epithelial DLD-1 cells (HLA-A*2402 positive, MAGE-A negative), further also referred to as target cells, were seeded and allowed to attach to the culture plate overnight at 37° C. with 5% CO₂. After 16 hours, the cell culture medium was refreshed and peripheral blood mononuclear cells (PBMCs), further referred to as effector cells, were added at a target to effector ratio 1:8 in presence or absence of 5 ng/ml 9A7×CD3 (SEQ ID NO: 65). The cells were further incubated for up to 16 and 72 hours at 37° C. with 5% CO₂. After each incubation, both target and effector cells were harvested. A flow cytometric analysis was performed in order to detect expression of T-cell activation markers (CD69 (Milteny biotec, cat. no 130-113-524) and CD25 (Milteny biotec, cat. no 130-113-282)) and lysosomal-associated membrane protein-1 (CD107a) (Milteny biotec, cat. no 130-111-621) on CD4 and CD8 positive cells (identified using Milteny biotec, cat. no. 130-113-22 and 130-110-679). Results are plotted as percentage of CD4 and CD8 positive cells expressing CD69, CD25 or CD107a and are shown in FIG. 23A, B for 16 hour incubation and 23° C., D for 72 hour incubation.

After 16 hours of incubation, both CD4 and CD8 positive cells show an increase in CD69, CD25 and CD107a expression when incubated together with HLA-A*2402 and MAGE-A positive H1299 cells in combination with 9A7×CD3 (SEQ ID NO: 65). When U118 served as target cell a potent induction of CD69 was observed on both CD4 and CD8 T cells in combination with increased levels of CD107a on CD8 positive cells.

HLA-A*2402 positive, MAGE-A negative cell line DLD-1 in combination with 9A7×CD3 (SEQ ID NO: 65) and PBMCs did not yield in increased expression of T cell activation markers. After 72 hours of incubation the most potent increase of T cell activation markers CD69 and CD25 were observed using the HLA-A*2402 and MAGE-A positive cell lines H1299 and U118. T cell activation marker CD69 was also induced on both CD4 and CD8 positive cells by 9A7×CD3 (SEQ ID NO: 65) when DLD-1 cells were used as target cell line. Lysosomal marker CD107a was only increased using U118 cells as target after 72 hour.

### Example 24

Immune Effector Cells Secrete Pro-Inflammatory Cytokines when Co-Cultured with Cells Expressing HLA-A*2402 and MAGE-A in the Presence of 9A7×CD3.

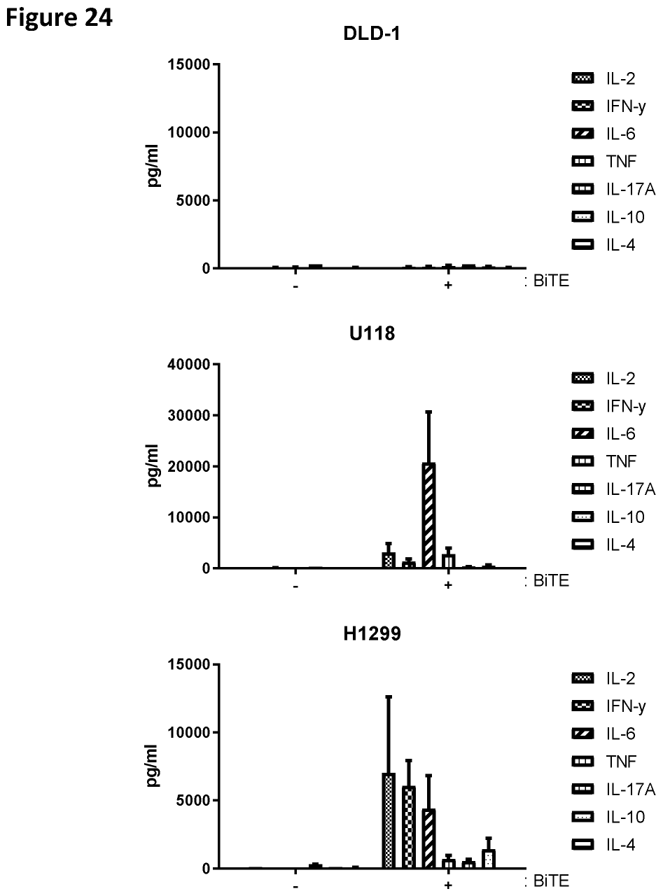

Following the demonstration of the expression of specific T cell activation markers in presence of 9A7×CD3 (SEQ ID NO: 65) and cells expressing HLA-A*2402 and MAGE-A, the cytokines released by T cells were measured using BD Cytometric Bead Array (CBA) Human Th1/Th2/Th17 Cytokine Kit (BD Biosciences, cat. no. 560484). In short, non small cell lung carcinoma H1299 (HLA-A*2402 and MAGE-A positive), brain glioblastoma U118 (HLA-A*2402 and MAGE-A positive) as well as colon epithelial DLD-1 cells (HLA-A*2402 positive, MAGE-A negative) were seeded in such a manner that 90-95% confluency was reached after the indicated incubation time. Cells were allowed to recover for 16 hours, after which the appropriate medium was replaced containing peripheral blood mononuclear cells (PBMCs) in 1:8 target to effector ratio and 5 ng/ml 9A7×CD3 (SEQ ID NO: 65). Cells were incubated for 72 hours at 37° C. with 5% CO₂. At the end of the incubation period, the cell supernatants were collected and analysed by flow cytometry for presence of the following cytokines; Interleukin-2 (IL-2), Interleukin-4 (IL-4), Interleukin-6 (IL-6), Interleukin-10 (IL-10), Tumor Necrosis Factor (TNF), Interferon-γ (IFN-γ), and Interleukin-17A (IL-17A) according to instruction provided in the BD Cytometric Bead Array (CBA) Human Th1/Th2/Th17 Cytokine Kit. 9A7×CD3 (SEQ ID NO: 65) induced secretion of IL-2, IFN-γ, IL-6 when PBMCs were co-cultured with HLA-A*2402 and MAGE-A expressing cells (FIG. 24). Additional cytokine secretion was cell line dependent, in which the presence of H1299, that have a high MAGE-A expression, resulted in the secretion of IL-10 by the PBMCs. Co-culturing of U118 cells, that comprise MAGE-A expression to a lesser extent compared to H1299, resulted in the secretion of TNF. Co-culturing of target expressing cells with PBMCs in the presence of BiTE did not result in the induction of IL-17A and IL-4 secretion. In addition, no cytokine release was induced by 9A7×CD3 (SEQ ID NO: 65) when co-cultured with none MAGE-A expressing DLD-1 cells.

### Example 25

9A7×CD3 Induces Apoptosis of H1299 Cells, but not of U118 and DLD-1

Poly-ADP-ribose polymerases (PARP) is one of several known cellular substrates of activated caspases. Its cleavage by caspases is considered to be a hallmark of apoptosis. To test the apoptosis inducing efficacy of 9A7×CD3 (SEQ ID NO: 65), Non small cell lung carcinoma H1299 (HLA-A*2402 and MAGE-A positive), brain glioblastoma U118 (HLA-A*2402 and MAGE-A positive) as well as colon epithelial DLD-1 cells (HLA-A*2402 positive, MAGE-A negative), further referred to as target cells, were seeded and allowed to attach to the culture plate overnight at 37° C. with 5% CO₂. After which the cell culture medium was refreshed and peripheral blood mononuclear cells (PBMCs), further referred to as effector cells, were added at a target to effector ratio 1:8 in presence or absence of 5 ng/ml 9A7×CD3 (SEQ ID NO: 65). The cells were further incubated for up to 72 hours at 37° C. with 5% CO₂. After incubation, the cells were harvested using trypsinization and lysed on ice using lysis buffer (150 mM NaCl, 25 mM Tris-HCl, 10 mM EDTA, 1% Trition X-100, 1× Protease inhibitor cocktail). Western Blot was performed in order to detect cleaved PARP and beta-actin as a loading control. Following antibodies were used to detect the indicated proteins: rabbit-anti-cleaved PARP (Cell Signalling Technologies, cat. no. 5625S), goat-anti-rabbit-HRP (Santa Cruz, cat. no. SC-2004), mouse anti-beta actin (Abcam, cat. no. AB8226), goat-anti-mouse-HRP (Santa Cruz, cat. no SC-516102-CM). Antibody binding was imaged by Chemidoc Imaging System (BioRad).

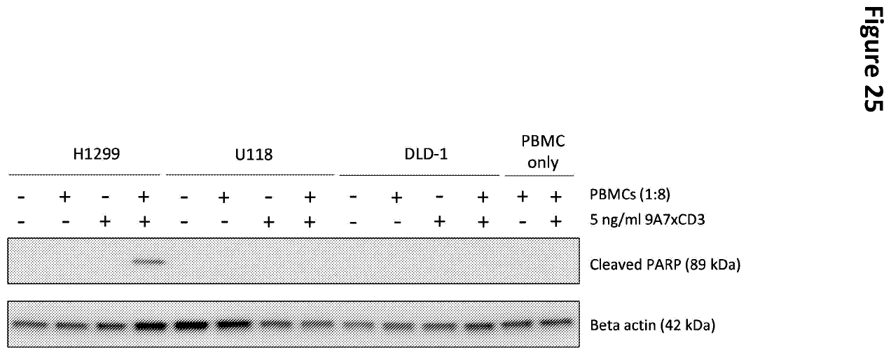

Apoptosis, as measured by the detection of cleaved PARP, was observed only in the MAGE-A and HLA-A*2402 positive H1299 cells when PMBCs and 9A7×CD3 (SEQ ID NO: 65) were combined (FIG. 25). None of the other cell lines or conditions demonstrated any indications of apoptosis.

### Example 26

4A6×CD3 BiTE Directs Human T Cells to Xenograft of H1299 Cells Stably Expressing HLA-A*0201/FLWGPRALV In Vivo.

Female hu-NSG mice (NOD.Cg-Prkdc^{scid}//2rg^{tm1Wjl/}SzJ), engrafted with human CD34+ cells, containing >25% human CD45+ were used. All experiments have been performed using human CD34+ cell engraftments of 3 different donors. Mice were housed in boxes to a maximum of 6 animals during acclimation period and to a maximum of 5 animals during the experimental phase. Each mouse was offered standard lab chow and filtered, sterilized tap water ad libitum throughout the study. 11 days before tumor cell engraftment 50 μl blood was drawn from each mouse to assess AAD, human CD45, CD3, CD4, CD8, CD69, CD25, CD107a and mouse CD45, in order to determine baseline levels of human T cells and their activation markers of each individual mouse. On the day of tumor graft induction, 249.3×10⁶ of H1299 cells stably expressing HLA-A*0201/FLWGPRALV derived cells were centrifugated at 450 g for 5 minutes at 4° C. and resuspended in cell culture medium (RPMI-1640 medium without FBS) to a final concentration of 50×10⁶ cells/ml. Just before injection, cell suspension was mixed 1:1 with Matrigel. Mice were anaesthetized with isoflurane and then skin was aseptized with alcohol. 100 μl of cell suspension, containing 2.5×10⁶ cells, were injected subcutaneously in the hind flank of the mice. After 4 days 5 mice per donor were allocated to the different treatment groups according to their respective baseline levels of humanization (CD45) and T cell levels (CD3). Dosing schedule consisted of 3 sequential daily injections, followed by a 4 day recovery period which was followed by a final 4 days of daily injections, of either vehicle, 5 μg or 20 μg 4A6×CD3 BiTE (SEQ ID NO: 63). 24 hours post final injection 50 μl blood was drawn from each mouse to assess AAD, human CD45, CD3, CD4, CD8, CD69, CD25, CD107a and mouse CD45. Mice treated with 20 μg 4A6×CD3 BiTE (SEQ ID NO: 63) showed a marked decrease in T cells present in the periphery, a similar drop was observed for CD19×CD3 during treatment of B-lineage acute lymphoblastic leukaemia patients by M Klinger et al in 2012. This drop in peripheral T cells was observed for both CD4 and CD8 T cell compartments.

Animals weight and tumor volume were measured 3 times a week. Animals were sacrificed when tumor volume was equal to or exceeded 2000 mm³ or when body weight loss exceeded 20% compared to study day 0. From all mice tumors were collected at ethical sacrifice for cytometry analysis of human immune infiltrated cells. Increased T cell infiltration compared to vehicle treated mice was observed in 2/3 donors treated with 4A6×CD3 BiTE (SEQ ID NO: 63). CD4 positive T cells mainly attributed to the increase in intra tumor T cells.

### Example 27

9A7×CD3 BiTE Directs and Activates Human T Cells to Xenograft of H1299 Cells Stably Expressing HLA-A*0201/FLWGPRALV In Vivo.

Female hu-NSG mice (NOD.Cg-Prkdc^{scid}//2rg^{tm1Wjl/}SzJ), engrafted with human CD34+ cells, containing >25% human CD45+ were used. All experiments have been performed using human CD34+ cell engraftments of 3 different donors. Mice were housed in boxes to a maximum of 6 animals during acclimation period and to a maximum of 5 animals during the experimental phase. Each mouse was offered standard lab chow and filtered, sterilized tap water ad libitum throughout the study. 11 days before tumor cell engraftment 50 μl blood was drawn from each mouse to assess AAD, human CD45, CD3, CD4, CD8, CD69, CD25, CD107a and mouse CD45, in order to determine baseline levels of human T cells and their activation markers of each individual mouse. On the day of tumor graft induction, 249.3×10⁶ of H1299 cells stably expressing HLA-A*0201/FLWGPRALV derived cells were centrifugated at 450 g for 5 minutes at 4° C. and resuspended in cell culture medium (RPMI-1640 medium without FBS) to a final concentration of 50×10⁶ cells/ml. Just before injection, cell suspension was mixed 1:1 with Matrigel. Mice were anaesthetized with isoflurane and then skin was aseptized with alcohol. 100 μl of cell suspension, containing 2.5×10⁶ cells, were injected subcutaneously in the hind flank. After 4 days 5 mice per donor were allocated to the different treatment groups according to their respective baseline levels of humanization (CD45) and T cell levels (CD3). Dosing schedule consisted of 3 days of sequential injections for all 3 donors, of either vehicle, 5 μg or 20 μg 9A7×CD3 BiTE (SEQ ID NO: 65). For one of the three donors this was followed by a 4 day recovery period followed by a final 4 days of daily injections, of either vehicle, 5 μg or 20 μg 9A7×CD3 BiTE (SEQ ID NO: 65). 24 hours post final injection, of the mice receiving the additional 4 doses, 50 μl blood was drawn from all mice to assess AAD, human CD45, CD3, CD4, CD8, CD69, CD25, CD107a and mouse CD45. Mice dosed with the additional 4 doses of either 5 μg or 20 μg 9A7×CD3 BiTE (SEQ ID NO: 65) showed a marked decrease in T cells present in the periphery, a similar drop was observed for CD19×CD3 during treatment of B-lineage acute lymphoblastic leukaemia patients by M Klinger et al in 2012. This drop in peripheral T cells was observed for both CD4 and CD8 T cell compartments.

Animals weight and tumor volume were measured 3 times a week. Animals were sacrificed when tumor volume was equal to or exceeded 2000 mm³ or when body weight loss exceeded 20% compared to study day 0. From all mice tumors were collected at ethical sacrifice for cytometry analysis of human immune infiltrated cells. Increased T cell infiltration compared to vehicle treated mice was observed in mice dosed with 5 μg (3/3 donors) or 20 μg (2/3 donors) 9A7×CD3 BiTE (SEQ ID NO: 65). T cells infiltrating the tumor upon dosing consisted of either CD4 or CD8 positive T cells, in a donor dependent manner, in mice dosed with 5 μg 9A7×CD3 BiTE (SEQ ID NO: 65). In the tumors demonstrating T cell infiltration upon dosing with 20 μg 9A7×CD3 BiTE (SEQ ID NO: 65) these T cells consisted of both CD4 and CD8 positive cells. For both concentrations' activation markers were found to be upregulated on tumor infiltrating T cells, which marker on which sub compartment of T cells varied per donor.

### Example 28

The Effect of Combined 4A6×CD3 and 9A7×CD3 on a Cell Transfected with their Respective Targets.

HEK 293-F cells are transfected 2 days prior to the efficacy testing to express HLA-*A2402 (HLA-A24) or HLA-A*0201 (HLA-A2) on the cell membrane. Pulsing of 200.000 HEK 293-F, with or without HLA-A24, HLA-A2 or a combination is performed overnight under serum free conditions with neither of target peptides (FLWGRPALV and IMPKTGFLI), or either of a single target peptide (FLWGRPALV or IMPKTGFLI), or combination of target peptides (FLWGRPALV and IMPKTGFLI). 100 μg/ml peptides as indicated in Table 3A (controls) and Table 3B are used to create both single and double target containing cells and their appropriate controls, as described in Example 22. Improved efficacy of the combination of 4A6×CD3 (SEQ ID NO: 63) and 9A7×CD3 (SEQ ID NO: 65) over each of the two single BiTE molecules is demonstrated using these cells as described in examples 23, 24 and 25 (See Table 3B). That is to say, either BiTE 4A6×CD3 (SEQ ID NO: 63) alone, or BiTE 9A7×CD3 (SEQ ID NO: 65) alone is efficacious in tumor cell killing, T-cell activation and/or when the cytokine release profile is considered, but improved T-cell activation, an improved cytokine release profile and/or improved cell-killing activity is achieved when target cells are targeted with the combination of two BiTEs (indicated with ‘improved’ in the Table 3B, 4, 5). Additionally, using a similar set-up as described in Example 18, K562 cells are used to overexpress both single and double HLA-A2 and HLA-24 MHC-I molecules in combination with and without 4A6×CD3 (SEQ ID NO: 63) and 9A7×CD3 (SEQ ID NO: 65) target overexpressing HLA-A2/FLWGPRALV and or HLA-A24/IMPKTGFLI, see Table 4. Improved efficacy of the combination of 4A6×CD3 (SEQ ID NO: 63) and 9A7×CD3 (SEQ ID NO: 65) over the single BITE is demonstrated using these cells as described in examples 23, 24 and 25 (See Table 4).

TABLE 3A
The effect of combined 4A6xCD3 and 9A7xCD3 on peptide pulsed HEK293F cells transfected
with HLA-A2 and or HLA-A24-controls

| | | | | | | | | | |
|---|---|---|---|---|---|---|---|---|---|
| Cells: | | | | | | | | | |
| MHC-I | No | No | No | No | HLA-A*0201 | HLA-A*0201 | HLA-A*2402 | HLA-A*2402 | HLA-A*0201 |
| | | | | | | | | | HLA-A*2402 |
| Peptide: FLWGPRALV | No | Yes | No | Yes | No | No | No | Yes | No |
| Peptide: IMPKTGFLI | No | No | Yes | Yes | No | Yes | No | No | No |
| Outome: | | | | | | | | | |
| 4A6xCD3: | | | | | | | | | |
| T-cell activation | No | No | No | No | No | No | No | No | No |
| Cytokine release | No | No | No | No | No | No | No | No | No |
| Target cell death | No | No | No | No | No | No | No | No | No |
| 9A7xCD3: | | | | | | | | | |
| T-cell activation | No | No | No | No | No | No | No | No | No |
| Cytokine release | No | No | No | No | No | No | No | No | No |
| Target cell death | No | No | No | No | No | No | No | No | No |
| 4A6xCD3 + 9A7xCD3: | | | | | | | | | |
| T-cell activation | No | No | No | No | No | No | No | No | No |
| Cytokine release | No | No | No | No | No | No | No | No | No |
| Target cell death | No | No | No | No | No | No | No | No | No |

TABLE 3B
The effect of combined 4A6xCD3 and 9A7xCD3 on peptide pulsed HEK296F cells transfected
with HLA-a2 and or HLA-A24 (See Table 3A for controls)

| | | | | | | | |
|---|---|---|---|---|---|---|---|
| Cells: | | | | | | | |
| MHC-I | HLA-A*0201 | HLA-A*0201 | HLA-A*2402 | HLA-A*2402 | HLA-A*0201 | HLA-A*0201 | HLA-A*0201 |
| | | | | | HLA-A*2402 | HLA-A*2402 | HLA-A*2402 |
| Peptide: FLWGPRALV | Yes | Yes | No | Yes | Yes | No | Yes |
| Peptide: IMPKTGFLI | No | Yes | Yes | Yes | No | Yes | Yes |
| Outome: | | | | | | | |
| 4A6xCD3: | | | | | | | |
| T-cell activation | Yes | Yes | No | No | Yes | No | Yes |
| Cytokine release | Yes | Yes | No | No | Yes | No | Yes |
| Target cell death | Yes | Yes | No | No | Yes | No | Yes |
| 9A7xCD3: | | | | | | | |
| T-cell activation | No | No | Yes | Yes | No | Yes | Yes |
| Cytokine release | No | No | Yes | Yes | No | Yes | Yes |
| Target cell death | No | No | Yes | Yes | No | Yes | Yes |
| 4A6xCD3 + 9A7xCD3: | | | | | | | |
| T-cell activation | Yes | Yes | Yes | Yes | Yes | Yes | Improved |
| Cytokine release | Yes | Yes | Yes | Yes | Yes | Yes | Improved |
| Target cell death | Yes | Yes | Yes | Yes | Yes | Yes | Improved |

TABLE 4
The effect of combined 4A6xCD3 and 9A7xCD3 MHC-I MAGE-A target complex over expressing K562 cells transfected.

| | | | | | | | |
|---|---|---|---|---|---|---|---|
| MHC-I: | — | HLA-A*0201 | — | HLA-A*0201 | HLA-A*0201/ | HLA-A*0201 | HLA-A*0201/ |
| | | | | | FLWGPRALV | | FLWGPRALV |
| | — | — | HLA-A*2402 | HLA-A*2402 | HLA-A*2402 | HLA-A*2402/ | HLA-A*2402/ |
| | | | | | | IMPKTGFLI | IMPKTGFLI |
| Outome: | | | | | | | |
| 4A6xCD3: | | | | | | | |
| T-cell activation | No | No | No | No | Yes | No | Yes |
| Cytokine release | No | No | No | No | Yes | No | Yes |
| Target cell death | No | No | No | No | Yes | No | Yes |
| 9A7xCD3: | | | | | | | |
| T-cell activation | No | No | No | No | No | Yes | Yes |
| Cytokine release | No | No | No | No | No | Yes | Yes |
| Target cell death | No | No | No | No | No | Yes | Yes |
| 4A6xCD3 + 9A7xCD3: | | | | | | | |
| T-cell activation | No | No | No | No | Yes | Yes | Improved |
| Cytokine release | No | No | No | No | Yes | Yes | Improved |
| Target cell death | No | No | No | No | Yes | Yes | Improved |

### Example 29

The Effect of Combined 4A6×CD3 and 9A7×CD3 on a Cell Natively Expressing their Respective Targets.

Cell lines natively expressing either neither, a single, or a combination of, HLA-A*0201 (HLA-A2), HLA-A*2402 (HLA-A24) and MAGE-A are assessed as described in Example 23, 24 and 25, for the efficacy of 4A6×CD3 (SEQ ID NO: 63), 9A7×CD3 (SEQ ID NO: 65) and the combination of both BiTE's, when tumor cell killing, T-cell activation and/or cytokine release profile is considered. These results and examples are demonstrating the improved efficacy of combinatory MHC-I/MAGE-A targeting, using 4A6×CD3 (SEQ ID NO: 63) and 9A7×CD3 (SEQ ID NO: 65), compared to single MHC-I/MAGE-A targeting, i.e. either 4A6×CD3 (SEQ ID NO: 63), or 9A7×CD3 (SEQ ID NO: 65). Experimental lay out-out and study outcome of these experiments is summarized in Table 5.

TABLE 5
The effect of combined 4A6xCD3 and 9A7xCD3 on a cell natively expressing their respective targets.

| | | | | | | | | |
|---|---|---|---|---|---|---|---|---|
| MHC-I: | — | HLA-A*0201 | — | HLA-A*0201 | — | HLA-A*0201 | — | HLA-A*0201 |
| | — | — | HLA-A*2402 | HLA-A*2402 | — | — | HLA-A*2402 | HLA-A*2402 |
| MAGE-A expression | — | — | — | — | yes | yes | yes | yes |
| Outome: | | | | | | | | |
| 4A6xCD3: | | | | | | | | |
| T-cell activation | No | No | No | No | No | Yes | No | Yes |
| Cytokine release | No | No | No | No | No | Yes | No | Yes |
| Target cell death | No | No | No | No | No | Yes | No | Yes |
| 9A7xCD3: | | | | | | | | |
| T-cell activation | No | No | No | No | No | No | yes | Yes |
| Cytokine release | No | No | No | No | No | No | yes | Yes |
| Target cell death | No | No | No | No | No | No | yes | Yes |
| 4A6xCD3 + | | | | | | | | |
| 9A7xCD3: | | | | | | | | |
| T-cell activation | No | No | No | No | No | Yes | yes | Improved |
| Cytokine release | No | No | No | No | No | Yes | yes | Improved |
| Target cell death | No | No | No | No | No | Yes | yes | Improved |

### FURTHER EMBODIMENTS OF THE INVENTION

A method for eradicating tumor cells expressing on their surface a MHC-peptide complex comprising a peptide derived from MAGE comprising contacting said cell with at least one immune effector cell through specific interaction of a specific binding molecule for said MHC-peptide complex.

A method as referred to above, wherein said specific binding molecule is a bispecific antibody.

A method as referred to above, wherein said specific binding molecule is a T cell receptor.

A method as referred to above, wherein said specific binding molecule is a chimeric antigen receptor.

A method as referred to above, wherein said specific binding molecule is associated with a T cell.

A bispecific antibody or molecule of which one arm specifically binds to a MHC-peptide complex comprising a peptide derived from MAGE associated with aberrant cells, and the other arm specifically recognizes a target associated with immune effector cells.

A bispecific antibody as referred to above, wherein said molecule comprises an immunoglobulin variable region.

A bispecific antibody as referred to above, wherein said immunoglobulin variable region comprises a VH, preferably in a BiTE format (e.g. two separate scFv connected by a linker (e.g. VL₁-VH₁-linker-VH₂-VL₂)).

A bispecific antibody as referred to above wherein said immunoglobulin variable region comprises a VHH (e.g. in the bispecific nanobody format such as: VHH-linker-VHH) A bispecific antibody as referred to above wherein said immunoglobulin variable region further comprises a VL.

A bispecific antibody as referred to above wherein said bispecific antibody is a human IgG, preferentially human IgG1, most preferably a human IgG wherein the Fc part does not activate the Fc receptor.

A bispecific antibody as referred to above, wherein the MHC-peptide complex comprises a peptide derived from MAGE, more preferably from MAGE-A, although the peptide can also be derived from MAGE-B or MAGE-C.

A bispecific antibody as referred to above, wherein said immune effector cells comprise T cells and NK cells.

A bispecific antibody as referred to above, wherein said target associated with an immune effector cell is CD3, CD16, CD25, CD28, CD64, CD89, NKG2D and/or NKp46, preferably CD3.

A bispecific antibody as referred to above for use in the treatment of cancer.

A T cell comprising a T cell receptor or a chimeric antigen receptor recognizing a MHC-peptide complex comprising a peptide derived from MAGE, more preferably from MAGE-A although the peptide can also be derived from MAGE-B or MAGE-C.

A method of producing a T cell as referred to above comprising introducing into said T cell nucleic acids encoding an α chain and a β chain or a chimeric antigen receptor.

A pharmaceutical composition comprising a bispecific antibody as referred to above and suitable diluents and/or excipients.

The bispecific antibody as referred to above, wherein the VH domain comprises SEQ ID NO: 1.

The bispecific antibody as referred to above, wherein the VH domain comprises SEQ ID NO: 2.

The bispecific antibody as referred to above, wherein the VH domain comprises SEQ ID NO: 61

The bispecific antibody as referred to above, wherein the VH domain is a human VH domain, a humanized VH domain or a camelid VH domain.

1. A bispecific molecule of which one arm comprises a first domain that specifically binds to a MHC-peptide complex comprising a peptide derived from MAGE expressed on the cell surface of aberrant cells, and the other arm comprises a second domain that specifically recognizes a target expressed on the cell surface of immune effector cells.

2. Bispecific molecule according to claim 1, wherein the first domain comprises a VH, VHH or VL.

3. Bispecific molecule according to claim 1, wherein said second domain comprises a VH, VHH, or VL.

4. Bispecific molecule according to claim 1, wherein the first domain is a VHH and/or the second domain is a VHH.

5. Bispecific molecule according to claim 1, wherein the molecule is a bispecific antibody.

6. Bispecific molecule according to claim 1, which is in a BiTE format.

7. Bispecific molecule according to claim 1, wherein the first domain binds specifically to a MHC/peptide complex comprising a peptide derived from MAGE-A.

8. Bispecific molecule according to claim 1, wherein the first domain is a VHH domain according to SEQ ID NO: 47; SEQ ID NO: 48; SEQ ID NO:49; SEQ ID NO: 50; SEQ ID NO: 51; SEQ ID NO: 52; SEQ ID NO: 53; SEQ ID NO: 54; SEQ ID NO: 55; or SEQ ID NO: 56.

9. Bispecific molecule according to claim 1, wherein the target recognized by the second domain is expressed on the cell surface of a T cell or NK-cell.

10. Bispecific molecule according to claim 1, wherein the target recognized by the second domain is CD3 or CD3 on a T-cell or NK-cell.

11. Bispecific molecule according to claim 1, wherein the second domain is a VHH domain according to SEQ ID NO: 57.

12. Bispecific molecule according to claim 1, wherein the first domain is a VHH domain according to SEQ ID NO: 47; SEQ ID NO: 48; SEQ ID NO:49; SEQ ID NO: 50; SEQ ID NO: 51; SEQ ID NO: 52; SEQ ID NO: 53; SEQ ID NO: 54; SEQ ID NO: 55; or SEQ ID NO: 56; and wherein the second domain is a VHH domain according to SEQ ID NO: 57.

13. A pharmaceutical composition comprising:
the bispecific molecule according to claim 1; and
suitable diluents and/or excipients.

14. A pharmaceutical composition according to claim 13, further comprising:
a further bispecific molecule of which one arm comprises a first domain that specifically binds to a MHC-peptide complex comprising a peptide derived from MAGE expressed on the cell surface of aberrant cells, and the other arm comprises a second domain that specifically recognizes a target expressed on the cell surface of immune effector cells, the further bispecific molecule having at least one different specificity compared to the first bispecific molecule.

15. A pharmaceutical composition according to claim 13, further comprising a second bispecific molecule of which one arm comprises a first domain that specifically binds to a MHC-peptide complex comprising a peptide derived from MAGE expressed on the cell surface of aberrant cells, and the other arm comprises a second domain that specifically recognizes a target expressed on the cell surface of immune effector cells, the further bispecific molecule having at least one different specificity compared to the first bispecific molecule.

16.-17. (canceled)

18. A method for eradicating tumor cells expressing on their surface a MHC-peptide complex comprising a peptide derived from MAGE, the method comprising:
contacting said tumor cells with at least one immune effector cell through specific interaction of a specific binding molecule for said MHC-peptide complex,
wherein said specific binding molecule is the bispecific molecule according to claim 1.

19. The method according to claim 18, wherein said tumor cells are contacted with at least one further bispecific molecule of which one arm comprises a first domain that specifically binds to a MHC-peptide complex comprising a peptide derived from MAGE expressed on the cell surface of aberrant cells, and the other arm comprises a second domain that specifically recognizes a target expressed on the cell surface of immune effector cells, said further bispecific molecule having at least one different specificity compared to the first bispecific molecule.

20. The method according to claim 18, wherein the VHH domain for binding to an MHC/MAGE peptide complex is according to SEQ ID NO: 47; SEQ ID NO: 48; SEQ ID NO:49; SEQ ID NO: 50; SEQ ID NO: 51; SEQ ID NO: 52; SEQ ID NO: 53; SEQ ID NO: 54; SEQ ID NO: 55; or SEQ ID NO: 56.

21. The method according to claim 18, wherein a VHH domain for binding to CD3 is according to SEQ ID NO: 57.

22. The method according to claim 18, wherein the binding molecule has a BiTE format.

23. The method according to claim 18, for the treatment of cancer, lung cancer, or non-small-cell lung cancer in a subject.


SEQUENCE LISTING (66 sequences)

SEQ ID NO: 1 - protein, 122 aa, Homo sapiens
EVQLVQSGAEVKKPGSSVKVSCKASGGTFSSYAISWVRQAPGQGLEWMGGIIPIFGTADYAQKFQGRATITADESTSTAYMELSSLRSEDTAVYYCARDYDFWSGYYAGDVWGQGTTVTVSS

SEQ ID NO: 2 - protein, 117 aa, Homo sapiens
QVQLVESGGGVVQPGRSLRLSCAASGFTFSTFPMHWVRQAPGKGLEWVAVIDYEGINKYYADSVKGRFTISRDNSKNTLYLQMNSLRAEDTAVYYCAGGSYYVPDYWGQGTLVTVSS

SEQ ID NO: 3 - protein, 117 aa, Homo sapiens
QLQLQESGGGVVQPGRSLRLSCAASGFTFSSFPMMWIRQAPGKGLEWVASISYDGSNKYYADSVKGRFTISRDNSKNTLYLQMNSLRAEDTAVYYCAGGSYYVPDYWGQGTLVTVSS

SEQ ID NO: 4 - protein, 117 aa, Homo sapiens
QLQLQESGGGVVQPGRSLRLSCAASGFTFSRNQMWWVRQAPGKGLEWVAVISIDQSVKYYADSVKGRFTISRDNSKNTLYLQMNSLRAEDTAVYYCAGGSYYVPDYWGQGTLVTVSS

SEQ ID NO: 5 - protein, 117 aa, Homo sapiens
QLQLQESGGGVVQPGRSLRLSCAASGFTFSTFPMHWVRQAPGKGLEWVAVIDYEGINKYYADSVKGRFTISRDNSKNTLYLQMNSLRAEDTAVYYCAGGSYYVPDYWGQGTLVTVSS

SEQ ID NO: 6 - protein, 117 aa, Homo sapiens
QLQLQESGGGVVQPGRSLRLSCAASGFTFSESAMHWVRQAPGKGLEWVAAISYDGSNKYYADSVKGRFTISRDNSKNTLYLQMNSLRAEDTAVYYCAGGSYYVPDYWGQGTLVTVSS

SEQ ID NO: 7 - protein, 117 aa, Homo sapiens
QLQLQESGGGVVQPGRSLRLSCAASGFTFSVFAMQWVRQAPGKGLEWVAAISYDGDNKYYADSVKGRFTISRDNSKNTLYLQMNSLRAEDTAVYYCAGGSYYVPDYWGQGTLVTVSS

SEQ ID NO: 8 - protein, 117 aa, Homo sapiens
QLQLQESGGGVVQPGRSLRLSCAASGFTFSERQMWWVRQAPGKGLEWVAVISNDTSSKYYADSVKGRFTISRDNSKNTLYLQMNSLRAEDTAVYYCAGGSYYVPDYWGQGTLVTVSS

SEQ ID NO: 9 - protein, 117 aa, Homo sapiens
QLQLQESGGGVVQPGRSLRLSCAASGFTFSERQMWWVRQAPGKGLEWVAVISHDGSTKYYADSVKGRFTISRDNSKNTLYLQMNSLRAEDTAVYYCAGGSYYVPDYWGQGTLVTVSS

SEQ ID NO: 10 - protein, 117 aa, Homo sapiens
QLQLQESGGGVVQPGRSLRLSCAASGFTFSSRQMWWVRPAPGKGLEWVAVISHDASAKYYADSVKGRFTISRDNSKNTLYLQMNSLRAEDTAVYYCAGGSYYVPDYWGQGTLVTVSS

SEQ ID NO: 11 - protein, 117 aa, Homo sapiens
QLQLQESGGGVVQPGRSLRLSCAASGFTFSVISMQWVRQAPGKGLEWVASISYDGSNKYYADSVKGRFTISRDNSKNTLYLQMNSLRAEDTAVYYCAGGSYYVPDYWGQGTLVTVSS

SEQ ID NO: 12 - protein, 117 aa, Homo sapiens
QLQLQESGGGVVQPGRSLRLSCAASGFTFSTFPMHWVRQAPGKGLEWVAAISYAGSNKYYADSVKGRFTISRDNSKNTLYLQMNSLRAEDTAVYYCAGGSYYVPDYWGQGTLVTVSS

SEQ ID NO: 13 - protein, 117 aa, Homo sapiens
QLQLQESGGGVVQPGRSLRLSCAASGFTFSTLPMHWVRQAPGKGLEWVAVISYNGENKYYADSVKGRFTISRDNSKNTLYLQMNSLRAEDTAVYYCAGGSYYVPDYWGQGTLVTVSS

SEQ ID NO: 14 - protein, 117 aa, Homo sapiens
QLQLQESGGGVVQPGRSLRLSCAASGFTFSTLPMHWVRQAPGKGLEWVAVISYDGSNKYYADSVKGRFTISRDNSKNTLYLQMNSLRAEDTAVYYCAGGSYYVPDYWGQGTLVTVSS

SEQ ID NO: 15 - protein, 117 aa, Homo sapiens
QLQLQESGGGVVQPGRSLRLSCAASGFTFSERQMWWVRQAPGKGLEWVAVISNDSSQKYYADSVKGRFTISRDNSKNTLYLQMNSLRAEDTAVYYCAGGSYYVPDYWGQGTLVTVSS

SEQ ID NO: 16 - protein, 117 aa, Homo sapiens
QLQLQESGGGVVQPGRSLRLSCAASGFTFSTMSMQWVRQAPGKGLEWVASISYDGSNKYYADSVKGRFTISRDNSKNTLYLQMNSLRAEDTAVYYCAGGSYYVPDYWGQGTLVTVSS

SEQ ID NO: 17 - protein, 117 aa, Homo sapiens
QLQLQESGGGVVQPGRSLRLSCAASGFTFSTLSMGWVRQAPGKGLEWVAWISYDGSNKYYADSVKGRFTISRDNSKNTLYLQMNSLRAEDTAVYYCAGGSYYVPDYWGQGTLVTVSS

SEQ ID NO: 18 - protein, 117 aa, Homo sapiens
QLQLQESGGGVVQPGRSLRLSCAASGFTFSTSAMQWVRQAPGKGLEWVAVIGYDGANKYYADSVKGRFTISRDNSKNTLYLQMNSLRAEDTAVYYCAGGSYYVPDYWGQGTLVTVSS

SEQ ID NO: 19 - protein, 117 aa, Homo sapiens; identical to SEQ ID NO: 14

SEQ ID NO: 20 - protein, 117 aa, Homo sapiens
QLQLQESGGGVVQPGRSLRLSCAASGFTFSSYAMHWVRQAPGKGLEWVAAISYDGRNKYYADSVKGRFTISRDNSKNTLYLQMNSLRAEDTAVYYCAGGSYYVPDYWGQGTLVTVSS

SEQ ID NO: 21 - protein, 117 aa, Homo sapiens
QLQLQESGGGVVQPGRSLRLSCAASGFTFSAGQMWWVRQAPGKGLEWVAVISHDESNKYYADSVKGRFTISRDNSKNTLYLQMNSLRAEDTAVYYCAGGSYYVPDYWGQGTLVTVSS

SEQ ID NO: 22 - protein, 117 aa, Homo sapiens
QLQLQESGGGVVQPGRSLRLSCAASGFTFSTYPMHWVRQAPGKGLEWVAVISYTGINKYYADSVKGRFTISRDNSKNTLYLQMNSLRAEDTAVYYCAGGSYYVPDYWGQGTLVTVSS

SEQ ID NO: 23 - protein, 117 aa, Homo sapiens
QLQLQESGGGVVQPGRSLRLSCAASGFTFSSRQMWWVRQAPGKGLEWVAVISHDASAKYYADSVKGRFTISRDNSKNTLYLQMNSLRAEDTAVYYCAGGSYYVPDYWGQGTLVTVSS

SEQ ID NO: 24 - protein, 117 aa, Homo sapiens
QLQLQESGGGVVQPGRSLRLSCAASGFTFSESAMHWVRQAPGKGLEWVAVISYSGMNKYYADSVKGRFTISRDNSKNTLYLQMNSLRAEDTAVYYCAGGSYYVPDYWGQGTLVTVSS

SEQ ID NO: 25 - protein, 117 aa, Homo sapiens; identical to SEQ ID NO: 21

SEQ ID NO: 26 - protein, 117 aa, Homo sapiens
QLQLQESGGGVVQPGRSLRLSCAASGFTFSESAMGWVRQAPGKGLEWVAWIGYDGQNKYYADSVKGRFTISRDNSKNTLYLQMNSLRAEDTAVYYCAGGSYYVPDYWGQGTLVTVSS

SEQ ID NO: 27 - protein, 117 aa, Homo sapiens
QLQLQESGGGVVQPGRSLRLSCAASGFTFSSQTMQWVRQAPGKGLEWVASISYDGENKYYADSVKGRFTISRDNSKNTLYLQMNSLRAEDTAVYYCAGGSYYVPDYWGQGTLVTVSS

SEQ ID NO: 28 - protein, 117 aa, Homo sapiens; identical to SEQ ID NO: 13

SEQ ID NO: 29 - protein, 117 aa, Homo sapiens
QLQLQESGGGVVQPGRSLRLSCAASGFTFSVQSMLWVRQAPGKGLEWVASIGYDGVNKYYADSVKGRFTISRDNSKNTLYLQMNSLRAEDTAVYYCAGGSYYVPDYWGQGTLVTVSS

SEQ ID NO: 30 - protein, 117 aa, Homo sapiens; identical to SEQ ID NO: 4

SEQ ID NO: 31 - protein, 117 aa, Homo sapiens
QLQLQESGGGVVQPGRSLRLSCAASGFTFSSFPMQWVRQAPGKGLEWVASIAYDGSNKYYADSVKGRFTISRDNSKNTLYLQMNSLRAEDTAVYYCAGGSYYVPDYWGQGTLVTVSS

SEQ ID NO: 32 - protein, 117 aa, Homo sapiens
QLQLQESGGGVVQPGRSLRLSCAASGFTFSMFAMHWVRQAPGKGLEWVAAISIDGSGKYYADSVKGRFTISRDNSKNTLYLQMNSLRAEDTAVYYCAGGSYYVPDYWGQGTLVTVSS

SEQ ID NO: 33 - protein, 117 aa, Homo sapiens
QLQLQESGGGVVQPGRSLRLSCAASGFTFSESPMFWVRQAPGKGLEWVAVISYTGYNKYYADSVKGRFTISRDNSKNTLYLQMNSLRAEDTAVYYCAGGSYYVPDYWGQGTLVTVSS

SEQ ID NO: 34 - protein, 117 aa, Homo sapiens
QLQLQESGGGVVQPGRSLRLSCAASGFTFSRHRMFWVRQAPGKGLEWVAGIGYWGWNKYYADSVKGRFTISRDNSKNTLYLQMNSLRAEDTAVYYCAGGSYYVPDYWGQGTLVTVSS

SEQ ID NO: 35 - protein, 117 aa, Homo sapiens
QLQLQESGGGVVQPGRSLRLSCAASGFTFSWRQMWWVRQAPGKGLEWVAVISHDGSGKYYADSVKGRFTISRDNSKNTLYLQMNSLRAEDTAVYYCAGGSYYVPDYWGQGTLVTVSS

SEQ ID NO: 36 - protein, 117 aa, Homo sapiens
QLQLQESGGGVVQPGRSLRLSCAASGFTFSSSQMWWVRQAPGKGLEWVAVISHDTSSKYYADSVKGRFTISRDNSKNTLYLQMNSLRAEDTAVYYCAGGSYYVPDYWGQGTLVTVSS

SEQ ID NO: 37 - protein, 117 aa, Homo sapiens
QLQLQESGGGVVQPGRSLRLSCAASGFTFSRQQMWWVRQAPGKGLEWVAVISLDPSIKYYADSVKGRFTISRDNSKNTLYLQMNSLRAEDTAVYYCAGGSYYVPDYWGQGTLVTVSS

SEQ ID NO: 38 - protein, 117 aa, Homo sapiens; identical to SEQ ID NO: 32

SEQ ID NO: 39 - protein, 117 aa, Homo sapiens
QLQLQESGGGVVQPGRSLRLSCAASGFTFSSIPMFWVRQAPGKGLEWVASISYNGENKYYADSVKGRFTISRDNSKNTLYLQMNSLRAEDTAVYYCAGGSYYVPDYWGQGTLVTVSS

SEQ ID NO: 40 - protein, 117 aa, Homo sapiens
QLQLQESGGGVVQPGRSLRLSCAASGFTFSESSMQWVRQAPGKGLEWVASIGYDGQNKYYADSVKGRFTISRDNSKNTLYLQMNSLRAEDTAVYYCAGGSYYVPDYWGQGTLVTVSS

SEQ ID NO: 41 - protein, 117 aa, Homo sapiens
QLQLQESGGGVVQPGRSLRLSCAASGFTFSVQSMQWVRQAPGKGLEWVAAIGYDGENKYYADSVKGRFTISRDNSKNTLYLQMNSLRAEDTAVYYCAGGSYYVPDYWGQGTLVTVSS

SEQ ID NO: 42 - protein, 117 aa, Homo sapiens; identical to SEQ ID NO: 6

SEQ ID NO: 43 - protein, 117 aa, Homo sapiens; identical to SEQ ID NO: 9

SEQ ID NO: 44 - protein, 117 aa, Homo sapiens
QLQLQESGGGVVQPGRSLRLSCAASGFTFSSFAMHWVRQAPGKGLEWVAVISYDGSNKYYADSVKGRFTISRDNSKNTLYLQMNSLRAEDTAVYYCAGGSYYVPDYWGQGTLVTVSS

SEQ ID NO: 45 - protein, 117 aa, Homo sapiens; identical to SEQ ID NO: 9

SEQ ID NO: 46 - protein, 117 aa, Homo sapiens
QLQLQESGGGVVQPGRSLRLSCAASGFTFSSLPMHWVRQAPGKGLEWVAAISYDGSNKYYADSVKGRFTISRDNSKNTLYLQMNSLRAEDTAVYYCAGGSYYVPDYWGQGTLVTVSS

SEQ ID NO: 47 - protein, 121 aa, Homo sapiens
EVQLVESGGGLVQHGGSLRLSCASSGTFFSINVMGWYRQAPGKQRDLFADISRSGNTNYADSVNGRFTISRDIAKNTVYLQMNSLKPEDTGVYYCYASADSHGRRVLTPYWGEGTQVTVSS

SEQ ID NO: 48 - protein, 121 aa, Homo sapiens
EVQLVESGGGLVQAGGSLRLSCTASGFTLDYKALVWFRQAPGKERERVSCISGGGGSTYYADSVKGRFTISRDNTKNTVYLQMNSLKPEDTAVYTCAAPQSFACTATLKYWGQGTQVTVSS

SEQ ID NO: 49 - protein, 125 aa, Homo sapiens
EVQLVESGGGLVHPGGSLRLSCAASGFTFSTYSMSWVRQAPGKGLEWVSSITPLGLSTKYGESVKGRFTISRDNAKKMLYLQMNSLKREDTAVYYCAKYPPGTQSITAKVRYDYRGQGTQVTVSS

SEQ ID NO: 50 - protein, 125 aa, Homo sapiens; identical to SEQ ID NO: 49

SEQ ID NO: 51 - protein, 116 aa, Homo sapiens
EVQQMEFGGGLVQNRGSRKLSCQASRMLFKVNTMGWYRQGPGKQSAWVADTTEGGSIKYADSVKGRFTISRDNAKTTAYLQKNSLKPEDTTLYFCNAIDRVNSQKWGQGTQVTVST

SEQ ID NO: 52 - protein, 116 aa, Homo sapiens
EVQLVESGGGLVQPGGSLKLSCQASRMLFKVNTMGWYRQGPGKQRELVADITEGGSIKYADSVKGRFTISRDNAKTTVYLQMNSLKPEDTAVYFCNAIDRVNSQYWGQGTQVTVSS

SEQ ID NO: 53 - protein, 116 aa, Homo sapiens; identical to SEQ ID NO: 52

SEQ ID NO: 54 - protein, 114 aa, Homo sapiens
EVQLVESGGTLVQPGGSLRLSCEASGFSFSTTHMSWVRQAPGKGLEWVARISSDGSRTTYADSVKGRFTISRDNAKNALYLQMNNLTFEDAAVYFCSTTITERRGRGTQVTVSS

SEQ ID NO: 55 - protein, 121 aa, Homo sapiens
EVQLVESGGGLVQHGGSLRLSCAASGTFFSINVMGWYRQAPGKQRDLVADISRTGNTNYADSVKGRFTISRDIAKNTVYLQMNSLKPEDTGVYYCYASAVSDGRRVLTPYWGEGTQVTVSS

SEQ ID NO: 56 - protein, 121 aa, Homo sapiens; identical to SEQ ID NO: 48

SEQ ID NO: 57 - protein, 117 aa, Homo sapiens
QVKLEESGGGLVQAGGLLRVSCTASGRTFDTMGWFRQAPGKEREFVAAVRWSSGNTLYGNTVKGRFTISRDTATNTVYLQMSSLKHEDTAVYYCAARWGGRGAADHWGQGTQVTVSS

SEQ ID NO: 58 - protein, 244 aa, Homo sapiens
EVQLVESGGGLVQPGGSLKLSCQASRMLFKVNTMGWYRQGPGKQRELVADITEGGSIKYADSVKGRFTISRDNAKTTVYLQMNSLKPEDTAVYFCNAIDRVNSQYWGQGTQVTVSSGGGGSQVKLEESGGGLVQAGGLLRVSCTASGRTFDTMGWFRQAPGKEREFVAAVRWSSGNTLYGNTVKGRFTISRDTATNTVYLQMSSLKHEDTAVYYCAARWGGRGAADHWGQGTQVTVSSHHHHHH

SEQ ID NO: 59 - protein, 110 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGGGTKLTVL

SEQ ID NO: 60 - protein, 111 aa, Homo sapiens
NFMLTQPHSVSESPGKTVTISCTRSSGSIASYYVQWYQQRPGSSPTTVISEDNQRPSGVPDRFSGSIDSSSNSASLTISGLKTEDEADYYCQSYDSSNVWVFGGGTKLTVL

SEQ ID NO: 61 - protein, 118 aa, Mus musculus
QVQLQQPGAELVKPGASVKLSCKASGYTFTSFWMHWVKQRPGQGLEWIGEIDSSDSYTNYNQKFKGKATLTVDKSSSTAYMQLSSLTSDDSAVYYCARRVGRGYFDYWGQGTTLTVSS

SEQ ID NO: 62 - protein, 107 aa, Mus musculus
DIVMTQSHKFMSTSVGDRVSITCKASQDVRTAVVWYQQKPGQSPKLLIYWASTRHTGVPDRFTGSGSGTDYILTISSVQAEDLALYYCQQYDTTPWTFGGGTKVEIK

SEQ ID NO: 63 - protein, 501 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGGGTKLTVLGGGGSGGGGSGGGGSEVQLVQSGAEVKKPGSSVKVSCKASGGTFSSYAISWVRQAPGQGLEWMGGIIPIFGTADYAQKFQGRATITADESTSTAYMELSSLRSEDTAVYYCARDYDFWSGYYAGDVWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 64 - protein, 497 aa, Homo sapiens
NFMLTQPHSVSESPGKTVTISCTRSSGSIASYYVQWYQQRPGSSPTTVISEDNQRPSGVPDRFSGSIDSSSNSASLTISGLKTEDEADYYCQSYDSSNVWVFGGGTKLTVLGGGGSGGGGSGGGGSQVQLVESGGGVVQPGRSLRLSCAASGFTFSTFPMHWVRQAPGKGLEWVAVIDYEGINKYYADSVKGRFTISRDNSKNTLYLQMNSLRAEDTAVYYCAGGSYYVPDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 65 - protein, 494 aa, Homo sapiens
DIVMTQSHKFMSTSVGDRVSITCKASQDVRTAVVWYQQKPGQSPKLLIYWASTRHTGVPDRFTGSGSGTDYILTISSVQAEDLALYYCQQYDTTPWTFGGGTKVEIKGGGGSGGGGSGGGGSQVQLQQPGAELVKPGASVKLSCKASGYTFTSFWMHWVKQRPGQGLEWIGEIDSSDSYTNYNQKFKGKATLTVDKSSSTAYMQLSSLTSDDSAVYYCARRVGRGYFDYWGQGTTLTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 66 - protein, 119 aa, Homo sapiens
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS